SEQUENCE LISTING (8 sequences)

SEQ ID NO: 1 - DNA, 1554 bp, synthetic construct
  misc_feature 1..1554: note = anti-fluorescein CAR
  source 1..1554: mol_type = other DNA
atggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 2 - protein, 517 aa, synthetic construct
  REGION 1..517: note = anti-fluorescein CAR
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1560 bp, synthetic construct
  misc_feature 1..1560: note = anti-fluorescein CAR
  source 1..1560: mol_type = other DNA
gccaccatggccttaccagtgaccgccttgctcctgccgctggccttgctgctccacgccgccaggccggatgtcgtgatgacccagacccccctcagcctcccagtgtccctcggtgaccaggcttctattagttgcagatccagccagtccctcgtgcactctaacggtaatacctacctgagatggtatctccagaagcccggacagagccctaaggtgctgatctacaaagtctccaaccgggtgtctggagtccctgaccgcttctcagggagcggttccggcaccgacttcaccctgaagatcaaccgggtggaggccgaagacctcggcgtctatttctgctctcagagtacacatgtgccctggaccttcggcggagggaccaagctggagatcaaaagctccgcagacgatgccaagaaagatgccgctaagaaagacgatgctaagaaagacgatgcaaagaaagacggtggcgtgaagctggatgaaaccggaggaggtctcgtccagccaggaggagccatgaagctgagttgcgtgaccagcggattcacctttgggcactactggatgaactgggtgcgacagtccccagagaaggggctcgaatgggtcgctcagttcaggaacaaaccctacaattatgagacatactattcagacagcgtgaagggcaggtttactatcagtagagacgattccaaatctagcgtgtacctgcagatgaacaatctcagggtcgaagatacaggcatctactattgcacaggggcatcctatggtatggagtatctcggtcaggggacaagcgtcacagtcagtttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - DNA, 3198 bp, synthetic construct
  misc_feature 1..3198: note = E2 anti-fluorescein CAR
  source 1..3198: mol_type = other DNA
atgcttctcctggtgacaagccttctgctctgtgagttaccacacccagcattcctcctgatcccaagcgtgctgacacagcctagctccgtgtctgccgcccctggccagaaagtgaccatcagctgtagcggcagcaccagcaacatcggcaacaactacgtgtcctggtatcagcagcaccccggcaaggcccccaagctgatgatctacgacgtgtccaagcggcccagcggcgtgcccgatagattttccggcagcaagagcggcaacagcgccagcctggatatcagcggcctgcagtctgaggacgaggccgactactattgcgccgcctgggacgatagcctgagcgagttcctgtttggcaccggcaccaagctgacagtgctgggcggaggcggaggatctggcggcggaggaagtggcggagggggatctcaggtgcagctggtggaaagcggcggcaacctggtgcagcctggcggatctctgagactgagctgtgccgccagcggcttcaccttcggcagcttcagcatgagctgggtgcgccaggctcctgggggaggactggaatgggtggcaggactgagcgccagaagcagcctgacccactacgccgatagcgtgaagggccggttcaccatcagccgggacaacgccaagaacagcgtgtacctgcagatgaacagcctgcgggtggaagataccgccgtgtactactgcgccagacggtcctacgacagcagcggctactggggccacttctacagctacatggacgtgtggggccagggcaccctcgtgacagtgtctgagagcaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 5 - protein, 1065 aa, synthetic construct
  REGION 1..1065: note = E2 anti-fluorescein CAR
MLLLVTSLLLCELPHPAFLLIPSVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 6 - DNA, 3207 bp, synthetic construct
  misc_feature 1..3207: note = 4M5.3 anti-fluorescein CAR
  source 1..3207: mol_type = other DNA
atgcttctcctggtgacaagccttctgctctgtgagttaccacacccagcattcctcctgatcccagacgttgtaatgacccagacccctctgtctctccccgtaagcttgggcgaccaggcgagcatctcttgtcggtcttcccagtccctggtccattcaaacggcaatacttacttgcggtggtacttgcagaagcccggtcaatccccaaaagtgctgatatacaaggttagcaatcgggtcagtggagtgcccgaccgcttcagcggaagcggatccgggactgacttcactctgaagatcaaccgggtagaagctgaagacctgggggtgtacttctgctctcagtcaacacacgtgccatggacctttggaggtggcaccaagctggaaatcaaatcatcagcggacgatgccaaaaaagacgcggccaagaaggacgatgccaagaaggatgatgctaaaaaggatggcggagtcaaattggacgagacaggcgggggactggtgcagcccggcggtgccatgaaactgtcttgtgtgaccagcggctttaccttcgggcattattggatgaactgggtgcgacagtctccagagaaagggctcgagtgggtggcccagtttcgaaataaaccgtacaattatgagacctactattcagattctgtgaaagggcgcttcactatttcacgcgacgacagcaaaagttccgtctaccttcagatgaacaaccttagagtggaggataccggaatatactactgcacgggtgccagttatggcatggagtacttggggcaggggacatctgtgaccgtttctgagagcaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 7 - protein, 1068 aa, synthetic construct
  REGION 1..1068: note = 4M5.3 anti-fluorescein CAR
MLLLVTSLLLCELPHPAFLLIPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - DNA, 2052 bp, synthetic construct
  misc_feature 1..2052: note = E2 anti-fluorescein CAR
  source 1..2052: mol_type = other DNA
agcgtgctgacacagcctagctccgtgtctgccgcccctggccagaaagtgaccatcagctgtagcggcagcaccagcaacatcggcaacaactacgtgtcctggtatcagcagcaccccggcaaggcccccaagctgatgatctacgacgtgtccaagcggcccagcggcgtgcccgatagattttccggcagcaagagcggcaacagcgccagcctggatatcagcggcctgcagtctgaggacgaggccgactactattgcgccgcctgggacgatagcctgagcgagttcctgtttggcaccggcaccaagctgacagtgctgggcggaggcggaggatctggcggcggaggaagtggcggagggggatctcaggtgcagctggtggaaagcggcggcaacctggtgcagcctggcggatctctgagactgagctgtgccgccagcggcttcaccttcggcagcttcagcatgagctgggtgcgccaggctcctgggggaggactggaatgggtggcaggactgagcgccagaagcagcctgacccactacgccgatagcgtgaagggccggttcaccatcagccgggacaacgccaagaacagcgtgtacctgcagatgaacagcctgcgggtggaagataccgccgtgtactactgcgccagacggtcctacgacagcagcggctactggggccacttctacagctacatggacgtgtggggccagggcaccctcgtgacagtgtctgagagcaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggc

(19) United States
(12) Patent Application Publication
(10) Pub. No.: US 2026/0263517 A1
(43) Pub. Date: Sep. 10, 2026

(54) THERAPEUTIC METHODS WITH TARGET-LINKED SMALL MOLECULES AND ANTI-TARGET CAR T CELLS

(71) Applicant: Seattle Children's Hospital (dba Seattle Children's Research Institute), Seattle, WA (US)

(72) Inventors: Michael C. Jensen, Seattle, WA (US); Julie Park, Seattle, WA (US)

(21) Appl. No.: 19/121,330

(22) PCT Filed: Oct. 17, 2023
(86) PCT No.: PCT/US2023/077070

§ 371 Date: Apr. 15, 2025

Related U.S. Application Data
(60) Provisional application No. 63/380,124, filed on Oct. 19, 2022.

(51) Int. Cl. A61K 35/17; A61K 9/00; A61K 31/519; A61K 40/11; A61K 40/31; A61K 40/40; A61K 47/54; A61P 35/00; C07K 16/00

(52) U.S. Cl. CPC A61K 35/17; A61K 9/0019; A61K 31/519; A61K 40/11; A61K 40/31; A61K 40/429; A61K 47/545; A61P 35/00; C07K 16/00; A61K 2239/38; A61K 2239/46; C07K 2317/21; C07K 2317/24; C07K 2317/622

(57) ABSTRACT

Some embodiments of the methods and compositions provided herein include therapies for treating, ameliorating or inhibiting a cancer, such as an osteosarcoma, in a human subject. Some embodiments include administration to the subject a fluorescein isothiocyanate (FITC)-folate conjugate, or a pharmaceutically acceptable salt thereof in an escalating dosing regimen, subsequent to or concurrently with administration to the subject of a chimeric antigen receptor (CAR) T cell therapy, such as a T cell comprising an anti-fluorescein CAR. In some such embodiments, the escalating dosing regimen comprises a dosing escalation cycle to identify a maximum tolerated dose (MTD) of the FITC-folate conjugate, or a pharmaceutically acceptable salt thereof.

### CROSS-REFERENCE TO RELATED APPLICATIONS

This application claims priority to U.S. Prov. App. No. 63/380,124 filed Oct. 19, 2022 which is incorporated by reference herein in its entirety.

### REFERENCE TO SEQUENCE LISTING

The present application is being filed along with a Sequence Listing in electronic format. The Sequence Listing is provided as a file entitled SCRI458WOSEQLIST, created Oct. 11, 2023, which is approximately 25,372 bytes in size. The information in the electronic format of the Sequence Listing is incorporated herein by reference in its entirety.

### FIELD OF THE INVENTION

Some embodiments of the methods and compositions provided herein include therapies for treating, ameliorating or inhibiting a cancer, such as an osteosarcoma, in a human subject. Some embodiments include administration to the subject of a fluorescein isothiocyanate (FITC)-folate conjugate, or a pharmaceutically acceptable salt thereof in an escalating dosing regimen, subsequent to or concurrently with administration to the subject of a chimeric antigen receptor (CAR) T cell therapy, such as a T cell comprising an anti-fluorescein CAR. In some such embodiments, the escalating dosing regimen comprises a dosing escalation cycle to identify a maximum tolerated dose (MTD) of the FITC-folate conjugate, or a pharmaceutically acceptable salt thereof.

### BACKGROUND OF THE INVENTION

Immunotherapy based on adoptive transfer of lymphocytes (e.g., T cells) into a patient is a valuable therapy in the treatment of cancer and other diseases. Important advancements have been made in the development of immunotherapies based on adoptive transfer of lymphocytes. Among the many different types of immunotherapeutic agents, one of the most promising of the immunotherapeutic agents being developed is T cells expressing chimeric antigen receptors (CAR T cells). The chimeric antigen receptor (CAR) is a genetically engineered receptor that is designed to target a specific antigen, for example, a tumor antigen. This targeting can result in cytotoxicity against the tumor, for example, such that CAR T cells expressing CARs can target and kill tumors via the specific tumor antigens.

First generation CARs are composed of a recognition region, e.g., a single chain fragment variable (scFv) region derived from an antibody for recognition and binding to the antigen expressed by the tumor, and an activation signaling domain, e.g., the CD3ζ chain of T cells can serve as a T cell activation signal in CARs. Although CAR T cells have shown positive results in vitro, they have had limited success in eliminating disease (e.g., cancer) in clinical trials. One problem has been the inability to prolong activation and expand the CAR T cell population in vivo.

To address this problem, a co-stimulation domain (e.g., CD137, CD28 or CD134) has been included in second generation CARs to achieve prolonged activation of T cells in vivo. Addition of a co-stimulation domain enhances the in vivo proliferation and survival of T cells containing CARs, and initial clinical data have shown that such constructs are promising therapeutic agents in the treatment of diseases, such as cancer.

Although improvements have been made in CAR T cell therapies, several problems remain. First, ‘off-target’ toxicity may occur due to normal cells that express the antigen targeted by the CAR T cells (e.g., a tumor-associated antigen). Second, unregulated CAR T cell activation may be found where the rapid and uncontrolled elimination of diseased cells (e.g., cancer cells) by CAR T cells induces a constellation of metabolic disturbances, called tumor lysis syndrome, or cytokine release syndrome (CRS), which can be fatal to patients. Tumor lysis syndrome and CRS can result due to administered CAR T cells that cannot be easily regulated and are activated uncontrollably. Accordingly, although CAR T cells show great promise as a tool in the treatment of diseases, such as cancer, additional CAR T cell therapeutic protocols are needed that provide reduced off-target toxicity, and more precise control of CAR T cell activation.

### SUMMARY OF THE INVENTION

Some embodiments provided herein relate to therapies including compositions and methods for treating, ameliorating or inhibiting an osteosarcoma in a human subject, comprising administering to the subject a fluorescein isothiocyanate (FITC)-folate conjugate, or a pharmaceutically acceptable salt thereof, in a dosing regimen comprising: a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof. (b) a second dose of about 5% to about 50% of the full dose of the FITC-folate conjugate, or a pharmaceutically acceptable salt thereof, (c) a third dose of about 50% to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg; wherein the human subject has received or is receiving a chimeric antigen receptor (CAR) T cell composition comprising a population of T cells expressing an anti-fluorescein CAR or an anti-fluorescein derivative CAR, thereby treating, ameliorating, or inhibiting the osteosarcoma in the human subject. In some embodiments, the osteosarcoma is recurring or refractory. In some embodiments, the period of time between administering the first dose and administering the second dose is between about 1 to about 7 days. In some embodiments, the period of time between administering the first dose and administering the second dose is about 2 days. In some embodiments, the period of time between administering the second dose and administering the third dose is between about 1 to about 7 days. In some embodiments, the period of time between administering the second dose and administering the third dose is about 3 days. In some embodiments, the full dose of the FITC-folate conjugate, or the pharmaceutically acceptable salt thereof, is about 3.1×10⁻² mg/kg. In some embodiments, the method further comprises administering a fifth dose of the MTD of the FITC-folate conjugate, or a pharmaceutically acceptable salt thereof. In some embodiments, the method further comprises a restaging period after the end of the fifth dose, wherein the restaging period comprises: assessing the subject to determine if one or more criteria for receiving at least one subsequent dosing cycle of the FITC-folate conjugate, or the pharmaceutically acceptable salt thereof, is met, provided the subject is not administered the FITC-folate conjugate during the restaging period. In some embodiments, the subject meets one or more criteria, and wherein the subject is administered at least one subsequent dosing cycle comprising administering the MTD of the FITC-folate conjugate, or the pharmaceutically acceptable salt thereof, in a dosing interval of once every about 5-10 days for a duration of time. In some embodiments, the CAR comprises a fully humanized anti-fluorescein antibody or antigen binding fragment thereof. In some embodiments, the CAR comprises a fully humanized anti-fluorescein scFv. In some embodiments, the CAR comprises a fully humanized E2 anti-fluorescein scFv. In some embodiments, the CAR T cells express a cell surface selectable marker comprising a truncated EGFR (EGFRt) polypeptide. In some embodiments, the dose of the CAR T cell composition is between about 1×10⁵ cells/kg to about 1×10⁷ cells/kg. In some embodiments, the FITC-folate conjugate, or the pharmaceutically acceptable salt thereof, is administered intravenously.

Some embodiments include methods of treating, ameliorating or inhibiting an osteosarcoma in a human subject, comprising administering to the subject a fluorescein isothiocyanate (FITC)-folate conjugate, or a pharmaceutically acceptable salt thereof, in a dosing regimen comprising: (i) a dosing escalation cycle to identify a maximum tolerated dose (MTD) of the FITC-folate conjugate, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, and (c) a third dose of about 50% to about 500% of the full dose of the FITC-folate conjugate, or a pharmaceutically acceptable salt thereof, wherein the full dose of the FITC-folate conjugate or a pharmaceutically acceptable salt thereof, is about 1×10⁻² mg/kg to about 5×10⁻² mg/kg; and (ii) a stable dosing cycle comprising administering the MTD of the FITC-folate conjugate, or a pharmaceutically acceptable salt thereof, in a dosing interval of once every about 5-10 days for a duration of time; wherein the human subject has received or is receiving a CAR T cell composition comprising a population of T cells expressing an anti-fluorescein CAR, thereby treating, ameliorating or inhibiting the osteosarcoma in the human subject. In some embodiments, the osteosarcoma is recurring or refractory. In some embodiments, the period of time between administering the first dose and administering the second dose is between about 1 to about 7 days. In some embodiments, the period of time between administering the first dose and administering the second dose is about 2 days. In some embodiments, the period of time between administering the second dose and administering the third dose is between about 1 to about 7 days. In some embodiments, the period of time between administering the second dose and administering the third dose is about 3 days. In some embodiments, the full dose of the FITC-folate conjugate, or the pharmaceutically acceptable salt thereof, is about 3.1×10² mg/kg. In some embodiments, the method further comprises administering a fifth dose of the MTD of the FITC-folate conjugate, or a pharmaceutically acceptable salt thereof. In some embodiments, the method further comprises a restaging period after the end of the fifth dose, wherein the restaging period comprises: assessing the subject to determine if one or more criteria for receiving at least one subsequent dosing cycle of the FITC-folate conjugate, or the pharmaceutically acceptable salt thereof, is met, provided the subject is not administered the FITC-folate conjugate during the restaging period. In some embodiments, the subject meets one or more criteria, and wherein the subject is administered at least one subsequent dosing cycle comprising administering the MTD of the FITC-folate conjugate, or the pharmaceutically acceptable salt thereof, in a dosing interval of once every about 5-10 days for a duration of time. In some embodiments, the CAR comprises a fully humanized anti-fluorescein antibody or antigen binding fragment thereof. In some embodiments, the CAR comprises a fully humanized anti-fluorescein scFv. In some embodiments, the CAR comprises a fully humanized E2 anti-fluorescein scFv. In some embodiments, the CAR T cells express a cell surface selectable marker comprising a truncated EGFR (EGFRt) polypeptide. In some embodiments, the dose of the CAR T cell composition is between about 1×10⁵ cells/kg to about 1×10⁷ cells/kg. In some embodiments, the FITC-folate conjugate, or the pharmaceutically acceptable salt thereof, is administered intravenously.

Some embodiments include methods of treating, ameliorating or inhibiting a cancer in a human subject comprising: (i) administering to the subject a first dose of a fluorescein isothiocyanate (FITC)-folate conjugate, or a pharmaceutically acceptable salt thereof; (ii) administering to the subject a second dose of the FITC-folate conjugate, or the pharmaceutically acceptable salt thereof, wherein the second dose is higher than the first dose; (iii) administering to the subject a third dose of the FITC-folate conjugate, or the pharmaceutically acceptable salt thereof, wherein the third dose is higher than the second dose, wherein a maximum tolerated dose (MTD) of the FITC-folate conjugate, or a pharmaceutically acceptable salt thereof, is identified after the third dose; and (iv) administering to the subject a fourth dose of the MTD of the FITC-folate conjugate, or a pharmaceutically acceptable salt thereof; wherein the human subject has received or is receiving a CAR T cell composition comprising a population of T cells expressing an anti-fluorescein CAR, thereby treating, ameliorating or inhibiting the cancer in the human subject. In some embodiments, the cancer comprises an osteosarcoma.

Some embodiments include methods of treating, ameliorating or inhibiting an osteosarcoma in a human subject, comprising administering to the subject a fluorescein isothiocyanate (FITC)-folate conjugate, or a pharmaceutically acceptable salt thereof in combination with a CAR T cell therapy, wherein the FITC-folate conjugate is administered to the subject in an escalating dosing regimen subsequent to or concurrently with administration of the CAR T cell therapy to the subject, thereby treating the osteosarcoma in the human subject. In some embodiments, the escalating dosing regimen comprises: a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, (c) a third dose of about 50% to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg. In some embodiments, the escalating dosing regimen comprises: (i) a dosing escalation cycle to identify a maximum tolerated dose (MTD) of the FITC-folate conjugate, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, and (c) a third dose of about 50% to about 500% of the full dose of the FITC-folate conjugate, or a pharmaceutically acceptable salt thereof, wherein the full dose of the FITC-folate conjugate or a pharmaceutically acceptable salt thereof, is about 1×10⁻² mg/kg to about 5×10⁻² mg/kg; and (ii) a stable dosing cycle comprising administering the MTD of the FITC-folate conjugate, or a pharmaceutically acceptable salt thereof, in a dosing interval of once every about 5-10 days for a duration of time. In some embodiments, the CAR T cell therapy comprises administering to the subject a population of CAR T cells expressing an anti-fluorescein CAR.

Some embodiments include use of a fluorescein isothiocyanate (FITC)-folate conjugate, or a pharmaceutically acceptable salt thereof for treating, ameliorating or inhibiting an osteosarcoma in a human subject in combination with a CAR T cell therapy, wherein the FITC-folate conjugate is administered to the subject in an escalating dosing regimen subsequent to or concurrently with administration of the CAR T cell therapy to the subject. In some embodiments, the escalating dosing regimen comprises a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, (c) a third dose of about 50% to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg. In some embodiments, the escalating dosing regimen comprises: (i) a dosing escalation cycle to identify a maximum tolerated dose (MTD) of the FITC-folate conjugate, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, and (c) a third dose of about 50% to about 500% of the full dose of the FITC-folate conjugate, or a pharmaceutically acceptable salt thereof, wherein the full dose of the FITC-folate conjugate or a pharmaceutically acceptable salt thereof, is about 1×10⁻² mg/kg to about 5×10⁻² mg/kg; and (ii) a stable dosing cycle comprising administering the MTD of the FITC-folate conjugate, or a pharmaceutically acceptable salt thereof, in a dosing interval of once every about 5-10 days for a duration of time.

Some embodiments include use of a fluorescein isothiocyanate (FITC)-folate conjugate, or a pharmaceutically acceptable salt thereof for treating, ameliorating or inhibiting a cancer in a human subject in combination with a CAR T cell therapy, wherein the FITC-folate conjugate, or a pharmaceutically acceptable salt thereof is administered in a method comprising: (i) administering to the subject a first dose of the FITC-folate conjugate, or a pharmaceutically acceptable salt thereof; (ii) administering to the subject a second dose of the FITC-folate conjugate, or the pharmaceutically acceptable salt thereof, wherein the second dose is higher than the first dose; (iii) administering to the subject a third dose of the FITC-folate conjugate, or the pharmaceutically acceptable salt thereof, wherein the third dose is higher than the second dose, wherein a maximum tolerated dose (MTD) of the FITC-folate conjugate, or a pharmaceutically acceptable salt thereof, is identified after the third dose; and (iv) administering to the subject a fourth dose of the MTD of the FITC-folate conjugate, or a pharmaceutically acceptable salt thereof. In some embodiments, the cancer comprises an osteosarcoma. In some embodiments, the FITC-folate conjugate is administered to the subject in an escalating dosing regimen subsequent to or concurrently with administration of the CAR T cell therapy to the subject. In some embodiments, the CAR T cell therapy comprises administration to the subject a population of CAR T cells expressing an anti-fluorescein CAR.

Some embodiments include a kit comprising a container comprising a fluorescein isothiocyanate (FITC)-folate conjugate, or pharmaceutically acceptable salt thereof, and optionally a pharmaceutically carrier, wherein the kit comprises instructions for treating, ameliorating or inhibiting osteosarcoma in a subject that has received or is receiving a CAR T cell composition comprising CAR T cells, wherein treating, ameliorating or inhibiting comprises administering the FITC-folate conjugate in a dosing regimen comprising: a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, (c) a third dose of about 50% to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg, and wherein the CAR T cells express an anti-fluorescein CAR. In some embodiments, the container is a vial.

Some embodiments include a system for treating, ameliorating or inhibiting osteosarcoma in a subject with a fluorescein isothiocyanate (FITC)-folate conjugate, or pharmaceutically acceptable salt thereof in combination with a CAR T cell therapy, wherein the FITC-folate conjugate is administered to the subject in an escalating dosing regimen subsequent to or concurrently with administration of the CAR T cell therapy to the subject, the system comprising: (a) a first dose of about 0.5% to about 5% of a full dose of the FITC-folate conjugate, or a pharmaceutically acceptable salt thereof; (b) a second dose of about 5% to about 500% of the full dose of the FITC-folate conjugate, or a pharmaceutically acceptable salt thereof; (c) a third dose of about 500% to about 500% of the full dose of the FITC-folate conjugate, or a pharmaceutically acceptable salt thereof, wherein a maximum tolerated dose (MTD) of the FITC-folate conjugate, or a pharmaceutically acceptable salt thereof is identified after the third dose; and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg. In some embodiments, the system further comprises a population of CAR T cells expressing an anti-fluorescein CAR.

### BRIEF DESCRIPTION OF THE DRAWINGS

FIG. 1 shows a diagram of an exemplary dosing scheme according to the claimed method. In this exemplary scheme, E2 CAR T cell (CAR T cell expressing SEQ ID NO:04) administration occurs in week 1 on Monday and Thursday prior to administration of the small molecule ligand linked to the targeting moiety by a linker (e.g., EC17) beginning in week 2. In week 2, the small molecule ligand linked to the targeting moiety by a linker (e.g., EC17) is administered on Monday and Thursday, and then in week 3 on Monday using a dose escalation sequence (i.e., Sequence 1). There is no administration of the small molecule ligand linked to the targeting moiety by a linker (e.g., EC17) during week 3 on Tuesday to Sunday. Weeks 2 to 3 are referred to as “Cycle 1”. In week 4, the small molecule ligand linked to the targeting moiety by a linker (e.g., EC17) is administered on Monday and Thursday, and then in week 5 on Monday using a dose escalation sequence (i.e., Sequence 2). There is no administration of the small molecule ligand linked to the targeting moiety by a linker (e.g., EC17) during week 5 on Tuesday to Sunday. Weeks 4 to 5 are referred to as “Cycle 2”. In week 6, the small molecule ligand linked to the targeting moiety by a linker (e.g., EC17) is administered on Monday and Thursday, and then in week 7 on Monday using a dose escalation sequence (i.e., Sequence 3). There is no administration of the small molecule ligand linked to the targeting moiety by a linker (e.g., EC17) during week 7 on Tuesday to Sunday. Weeks 6 to 7 are referred to as “Cycle 3”. Weeks 2 to 7 are referred to as “Course 1”. During Sequence 1, the small molecule ligand linked to the targeting moiety by a linker (e.g., EC17) is administered in week 2 on Monday and Thursday, and then in week 3 on Monday with doses of the small molecule ligand linked to the targeting moiety by a linker (e.g., EC17) that are 1, 10, and 100 percent of a full dose (e.g., 31 μg/kg), respectively, of the small molecule ligand linked to the targeting moiety (e.g., EC17). During Sequence 2, the small molecule ligand linked to the targeting moiety by a linker (e.g., EC17) is administered in week 4 on Monday and Thursday, and then in week 5 on Monday with doses of the small molecule ligand linked to the targeting moiety by a linker (e.g., EC17) that are 1, 30, and 300 percent of a full dose (e.g., 31 μg/kg), respectively, of the small molecule ligand linked to the targeting moiety (e.g., EC17). During Sequence 3, the small molecule ligand linked to the targeting moiety by a linker (e.g., EC17) is administered in week 6 on Monday and Thursday, and then in week 7 on Monday with doses of the small molecule ligand linked to the targeting moiety by a linker (e.g., EC17) that are 1, 50, and 500 percent of a full dose (e.g., 31 μg/kg), respectively, of the small molecule ligand linked to the targeting moiety (e.g., EC17). Course 1 is then repeated assuming clinical benefit and/or tolerable toxicity.

FIG. 2 shows a general diagram of a construct used for CAR T cell transduction.

FIG. 3 shows an E2 construct vs. a 4M5.3 construct diagrammatically and shows a plasmid map of the E2 construct.

FIG. 4 (top panel) is a chart showing tumor volumes of HOS-FRα (human osteosarcoma-folate receptor alpha) tumors when treated with E2 Car-T cells only (•) and E2 Car-T cells+EC-17 (o). FIG. 4 (bottom panel) is a chart showing the body weight change in mice bearing HOS-FRα tumors when treated with E2 Car-T cells only (•) and E2 Car-T cells+EC-17 (o).

FIG. 5 is a chart showing the body weight change in mice bearing THP-1-FRβ (folate receptor beta) tumors expressing AMF (autocrine motility factor) when treated with no Car-T (•); no EC-17 (▪); EC-17 SIW 500 nmol/kg (Δ); EC-17 TIW (5, 50, 500 nmol/kg on/off) (∇); (♦) EC-17 dose escalation (M/Th/M on/off).

FIG. 6 (left panel) is a chart showing liver metastatic tumor burden. FIG. 6 (right panel) is a chart showing non-liver metastatic tumor burden.

FIG. 7 is a chart showing counts of circulating THP1-FRb cells per 100 μL blood at day 39 on a logarithmic scale.

FIG. 8 is a chart showing percentage of the total E2 CAR T cells isolated from the solid liver tumors (y axis) at different EC-17 dosing regimens. For each dosing regimen, far left bar is PD1+LAG3+ TIM3+, second left bar is PD1+LAG3+ TIM3−, middle bar is PD1+LAG3− TIM3+; second right bar is PD1+LAG3− TIM3−; far right bar is PD1− LAG3− TIM3−.

FIG. 9 shows a fully human CAR construct including anti-FITC scFv (clone E2), a full-length IgG4 spacer (Fc derived hinge-CH2(F235D, N297Q)-CH3), CD28tm transmembrane domain, 4-1BB/CD3ζ cytoplasmic activation domains, and a non-functional truncated cell surface polypeptide of epidermal growth factor receptor (EGFRt). Bottom: Examples of CD4/CD8 T cell phenotyping performed by flow cytometry on an EGFRt-sorted (left pie charts) CAR-T cell preparation and an unsorted “clinical facsimile” (right pie charts). The color keys are as shown.

FIG. 10, Panel A: Kd values of ³H-EC17 uptake by FR+ target cells after a 2-hour incubation at 37° C. (calculated from the numbers of molecules bound per cell). Panel B: Kd value of 3H-EC17 uptake by E2-CAR-T cells (−24% EGFRt+, −95:5 CD8/CD4 ratio) after a 2-hour incubation at room temperate (calculated from total cell-associated radioactivity, DPM).

FIG. 11 shows functional FR levels on tumor cells measured by a 3H-FA-based binding assay (100 nM, 1 hour at 37° C.).

FIG. 12 shows EC17 dose finding and CRS assessment in tumor versus tumor-free mice. Panel A: Schematic diagram to show dose scheduling of CAR-T cells (−10 million “clinical facsimile”) plus EC17 dosed 3 different ways in NSG mice without or with pre-established MDA-MB-231 xenografts. Tumor-free mice received EC17 SIW 500 nmol/kg (2 doses on days 2 and 10). Tumor-bearing mice received EC17 as follows: EC17 SIW 500 nmol/kg (5 doses on days 2, 10, 17, 24, and 31), EC17 M/Th/M_Escalation-1 (repeats of 5/50/500 nmol/kg on Monday/Thursday/Monday followed by 1-week break, i.e., on days 2, 6, 10, 17, 20, 24, and 31), or EC17 M/Th/M_Escalation-2 (repeats of 5/100/1000 nmol/kg on Monday/Thursday/Monday followed by 1-week break, i.e., days 2, 6, 10, 17, 20, 24, and 31). Panel B: Systemic levels of human IFNγ on a log 2 scale detected in mouse plasma on days 11 and 12 after CAR-T cell injection in tumor-bearing versus tumor-free mice (i.e., ~20 and 42 hours after the previous EC17 dose in all treated cohorts). Panel C: Circulating CAR-T cells in mouse blood identified as human CD3ε+ EGFRt+ events by flow cytometry and enumerated per 100 μL of whole blood. Panel D: Measurements of change in body weight and tumor volume in tumor-bearing mice that received CAR-T cells only or CAR-T cells plus EC17 dosed 3 different ways (dashed lines indicated each EC17 dose). n=5 mice per group. All data represent mean±s.e.m. * p<0.05 by one-way ANOVA test.

FIG. 13 shows EC17 dose escalation in safety and anti-leukemic activity in-vivo. Panel A: Schematic diagrams to show dose scheduling of EC17 plus unsorted EGFRt CAR-T cells (~6 million, day 0) in NSG mice with 1-day-old intravenous THP-1-FRβ) xenografts. Starting 3 days after CAR-T cell injection, EC17 was dosed in 3 different ways: EC17 SIW 500 nmol/kg (6 doses on days 3, 10, 17, 24, 31, and 38), EC17 TIW 5/50/500 nmol/kg (3 repeats of 5/50/500 nmol/kg on Monday/Wednesday/Friday followed by a 9-day break, i.e., on days 3/5/7, 17/19/21 and 31/33/35), or EC17 M/Th/M_dose escalation on Monday/Thursday/Monday (3 escalation cycles at 5/10/100 nmol/kg in Cycle 1, 5/30/300 nmol/kg in Cycle 2, and 5/50/500 nmol/kg in Cycle 3, i.e., on days 3/6/10, 17/20/24, and 31/34/38). Panel B: Measurements of change in body weight (n=5). Panel C: Circulating CAR-T cells as human CD3s+ EGFRt+ events found per 100 μL of mouse whole blood on a logarithmic scale on day 31. Panel D: Left bar graph: circulating tumor cells (GFP+) per 100 μL of whole blood in all cohorts measured at the end of study (day 39); Middle bar graph: THP1-ERB infiltrated liver weights representing liver metastatic burden; Right bar graph: total tumor weights of all non-liver macrometastases. Panel E: Flow cytometric analysis of T-cell exhaustion markers, PD1, LAG3, TIM3, on pre-infusion CAR-T cell product (triple-negative) and tumor-infiltrating CAR-T cells isolated from liver metastases. A cardinal feature of fully exhausted T cells is co-expression of multiple inhibitory receptor markers (i.e., triple-positive).

FIG. 14 shows antitumor activity and CRS rescue in an aggressive model of pediatric osteosarcoma. Panel A: Schematic diagram to show dose scheduling of CAR-T cells (~6 million, day 0) plus EC17 in NSG mice with 3-day-old subcutaneous HOS-FRα xenografts (n=5). Starting 3 days after CAR-T cell injection (6 days after tumor implant), 3 cycles of EC17 M/Th/M “intra-patient” dose escalation on Monday/Thursday/Monday at 5/10/100 nmol/kg in Cycle 1, 5/30/300 nmol/kg in Cycle 2, and 5/50/500 nmol/kg in Cycle 3, i.e. on days 3/6/10, 17/20/24, and 31/34/38. Panel B: Measurements of tumor volumes and change in body weights. Due to tumor progression, five mice received CAR-T cells only (no EC17) were euthanized on days 23-31, and five EC17 treated mice were euthanized on days 33 (2 mice) and 47 (3 mice), respectively. Panel C: Flow cytometric analysis of CAR-T cells (human CD3ε+EGFRt+) per 100 μL of whole blood plotted on a logarithmic scale (left) and tumor-infiltrating CAR-T cells at the time of euthanasia (right).

FIG. 15 depicts an experimental design scheme for a clinical trial.

FIG. 16 depicts a dose modification schema.

FIG. 17 depicts the dose methodology for Dose Regimen 0. Under Course 1 of this methodology, a patient is given a CAR T infusion at 1×10⁶ cells/kg at day 0, followed by the administration of an MTD determined dose of compound UB-TT170 in an escalation phase of 0.5%, 5%, or 50% of a full dose at days 4, 7, 11, 18, and 25. After this dose escalation, restaging will occur. Under Courses 2-4, the patient is administered an MTD determined dose of compound UB-TT170 in an escalation phase (0.5%, 5%, or 50% of a full dose) at days 1, 8, 15, 22, 29, 36, and 43. The full dose of UB-TT170 is 3.1×10² mg/kg.

FIG. 18 depicts the dose methodology for Dose Regimen 1. Under Course 1 of this methodology, a patient is given a CAR T infusion at 1×10⁶ cells/kg at day 0, followed by the administration of an MTD determined dose of compound UB-TT170 in an escalation phase of 1%, 10%, or 100% of a full dose at days 4, 7, 11, 18, and 25. After this dose escalation, restaging will occur. Under Courses 2-4, the patient is administered an MTD determined dose of compound UB-TT170 in an escalation phase (1%, 10%, or 100% of a full dose) at days 1, 8, 15, 22, 29, 36, and 43. The full dose of UB-TT170 is 3.1×10⁻² mg/kg.

FIG. 19 depicts the dose methodology for Dose Regimen 2. Under Course 1 of this methodology, a patient is given a CAR T infusion at 1×10⁶ cells/kg at day 0, followed by the administration of an MTD determined dose of compound UB-TT170 in an escalation phase of 1%, 30%, or 300% of a full dose at days 4, 7, 11, 18, and 25. After this dose escalation, restaging will occur. Under Courses 2-4, the patient is administered an MTD determined dose of compound UB-TT170 in an escalation phase (1%, 30%, or 300% of a full dose) at days 1, 8, 15, 22, 29, 36, and 43. The full dose of UB-TT170 is 3.1×10² mg/kg.

FIG. 20 depicts the dose methodology for Dose Regimen 3. Under Course 1 of this methodology, a patient is given a CAR T infusion at 1×10⁶ cells/kg at day 0, followed by the administration of an MTD determined dose of compound UB-TT170 in an escalation phase of 1%, 50%, or 500% of a full dose at days 4, 7, 11, 18, and 25. After this dose escalation, restaging will occur. Under Courses 2-4, the patient is administered an MTD determined dose of compound UB-TT170 in an escalation phase (1%, 50%, or 500% of a full dose) at days 1, 8, 15, 22, 29, 36, and 43. The full dose of UB-TT170 is 3.1×10² mg/kg.

FIG. 21 depicts an EC17 intra-host dose escalation treatment (3 cycles) which includes administration of about 5 million E2 CAR T cells at day −3; administration of EC17 at days 0, 3, 7, 14, 17, 21, 28, 31 and 35.

FIG. 22 depicts an EC17 intra-host dose escalation treatment (3 cycles).

FIG. 23A depicts a study schema for Group 2 (CAR-T only).

FIG. 23B depicts a study schema for Group 3 (EC17 SIW).

FIG. 23C depicts a study schema for Group 4 (EC17 true TIW).

FIG. 23D depicts a study schema for Group 5 (EC17 low-dose “TIW” (M/Th/M, 6-day off) with/efficacy).

FIG. 24A depicts an EC17 dosing regimen for Cohort 3.

FIG. 24B depicts an EC17 dosing regimen for Cohort 4.

FIG. 24C depicts an EC17 dosing regimen for Cohort 5.

### DETAILED DESCRIPTION

Survival after cancer during childhood and adolescence has improved dramatically in the last several decades, with the greatest reduction in mortality observed in patients with hematologic malignancies. The treatment of pediatric and young adult solid tumors remains challenging despite the employment of aggressive multimodal therapy. Aside from primary CNS malignancies, the most common diagnoses in this demographic include neuroblastoma, bone and soft tissue sarcomas, and renal tumors. Recent attempts to improve outcomes with intensification of cytotoxic therapies have yielded limited success. Patients with the highest risk disease, i.e., those who present with metastatic, relapsed or refractory disease, are particularly resistant to such strategies, and their outcomes continue to be poor. Prognosis for patients with recurrent osteosarcoma is poor. Subjects enrolled on an upfront Phase 3 international trial INT-0133 who experienced recurrence had an overall survival rate of 20% at 10 years, which mirrors outcomes reported by other groups. Although there are several agents with activity in recurrent or refractory osteosarcoma, there are no chemotherapy agents or targeted therapies proven to improve overall survival in patients with recurrent osteosarcoma. Therefore, therapeutic options for this group of patients is extremely limited. Surgical resection via aggressive thoracotomies are generally accepted to be standard treatment for resectable osteosarcoma recurrences limited to the lung. Patients who develop bony or other sites of visceral recurrence (for example in the brain) also have very poor prognosis, often because surgical intervention is not feasible. Survivors from all risk groups frequently face chronic and debilitating long term complications of therapy, including infertility, cardiac toxicity and second malignant neoplasms. Therefore, new approaches that target the pathobiology of these diseases must be explored.

The potential of targeting tumor burden with immune-based therapeutics is attractive because of the opportunity to invoke immunologic effector mechanisms to which chemotherapy/radiation-resistant tumor cells are susceptible, and because of limited toxicity theoretically possible with tumor-specific immunologic effector mechanisms. Ideally, a cell-surface epitope target would be expressed solely on tumor cells and uniformly on all tumors within one or more specific population(s) of patients. To date, few ideal tumor-specific epitopes have been defined. The approach that has supplied the most experience to date in pediatric solid tumors is the use of tumor-directed monoclonal antibody therapy, which can target growth factors as well as other cell surface and immunomodulatory molecules. A related strategy is the adoptive transfer of T-lymphocytes that have been engineered to recognize tumor antigens with the specificity of a monoclonal antibody through the expression of a chimeric antigen receptor (CAR). T cells expressing tumor antigen-specific CARs specifically lyse target tumor cells and secrete pro-inflammatory cytokines. The recent success of CD19-specific CAR T cell therapy in high-risk B-cell malignancies has prompted interest in investigating this approach in solid tumors. Clinical trials using CAR T cells directed against L1-CAM, GD-2 in neuroblastoma and HER2 in sarcoma have provided some promising initial reports.

While the adoptive transfer of tumor-specific T cells may result in tumor eradication in a variety of animal models, adoptive T cell therapy for human malignancy has been significantly more challenging and less effective than for viral diseases. Perhaps the most clinically robust adoptive therapy data comes from efforts of the Surgery Branch at the National Cancer Institute (NCI) to treat melanoma. Their studies demonstrated that administration of lymphodepleting chemotherapy followed by the adoptive transfer of polyclonal tumor infiltrating lymphocytes (TILs) specific for melanocyte lineage antigens/tumor-testis antigens and high-dose rhuIL-2, results in demonstrable tumor regressions in nearly one half of treated subjects. Of note, the response rate in subjects that exhibited high-level persistent engraftment of transferred T cells was nearly 90%, while no responders were seen in the approximately 50% of subjects without T cell engraftment. Interestingly, persisting TILs represented only a small subset of the infused product repertoire, suggesting that the capacity for engraftment and persistence may be linked to the intrinsic programming of a rare population of T cells derived from TIL. The generalization of the NCI group's adoptive transfer results for melanoma to other tumor types is the subject of intense ongoing investigation.

Known barriers to the clinical success of adoptive therapy: Tumor antigens presented by human leukocyte antigen (HLA) molecules and recognized by T cells have been identified and can originate from processing of normal or over-expressed tissue-specific proteins, aberrantly expressed onco-fetal antigens and mutated proteins, or minor histocompatibility antigens in the setting of allogeneic HCT. However, isolating and expanding high-affinity major histocompatibility complex (MHC)-restricted tumor-reactive T cells from tumor-bearing subjects is technically difficult and even when successful, these T cells often fail to eliminate tumors after adoptive transfer. Our evolving understanding of the counter-regulatory mechanisms that impede successful tumor therapy have been derived largely from murine models. Because many tumor antigens are self-proteins that are expressed in some normal tissues, tolerance mechanisms shape the frequency, avidity, and function of tumor-reactive T cells. Additionally, tumors evade immune recognition through a variety of mechanisms including local recruitment of regulatory T cells (Treg) and other suppressor cells, loss of antigen expression, down-regulation of MHC and costimulatory molecules, and expression or secretion of inhibitory molecules or cytokines. Finally, cultured tumor-reactive T cells often exhibit limited persistence in vivo after adoptive transfer, even if high doses of rhuIL-2 are administered to support their survival.

Some embodiments of the methods and compositions provided herein include therapies for treating, ameliorating or inhibiting a cancer, such as an osteosarcoma, in a human subject. Some embodiments include administration to the subject of a fluorescein isothiocyanate (FITC)-folate conjugate, or a pharmaceutically acceptable salt thereof in an escalating dosing regimen, subsequent to or concurrently with administration to the subject of a chimeric antigen receptor (CAR) T cell therapy, such as a T cell comprising an anti-fluorescein CAR. In some such embodiments, the escalating dosing regimen comprises a dosing escalation cycle to identify a maximum tolerated dose (MTD) of the FITC-folate conjugate, or a pharmaceutically acceptable salt thereof. Some embodiments of the methods and compositions provided herein include aspects disclosed in U.S. 2021/0346431 which is expressly incorporated by reference herein in its entirety.

In the various embodiments described herein, a small molecule ligand linked to a targeting moiety by a linker is used as a bridge between a cancer and chimeric antigen receptor (CAR) T cells which are T cells expressing a CAR. The bridge directs the CAR T cells to the cancer for amelioration of the cancer. In one embodiment, the “small molecule ligand” can be a folate, a CAIX ligand, DUPA, an NK-1R ligand, a ligand of gamma glutamyl transpeptidase, an NKG2D ligand, or a CCK2R ligand, each of which is a small molecule ligand that binds specifically to a cancer cell type (i.e., the receptor for each of these ligands is overexpressed on cancers compared to normal tissues).

In some embodiments, the “small molecule ligand” is an NK-1 receptor ligand. In these embodiments, the NK-1 receptor-binding moiety may be any of a number of ligands, such as a small molecule ligand customized to be highly selective or specific for an NK-1 receptor. An example NK-1 receptor-binding ligand (“NKIRL”) may be synthesized from a high affinity NK-1 receptor antagonist (2S,3S)-3-{[3,5 -bis(trifluoromethyl)benzyl]oxy}-2-phenylpiperidine (“L733060”) and acetic acid (AcOH), and has the formula:

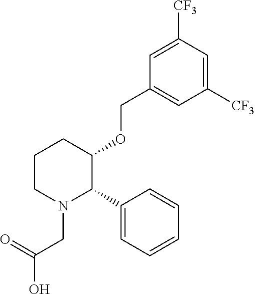 [SMILES: O=C(O)CN1CCC[C@H](OCc2cc(C(F)(F)F)cc(C(F)(F)F)c2)[C@@H]1c1ccccc1]

In alternative embodiments of fluorescent imaging conjugates, the NK-1 receptor-binding moiety may be a small molecule ligand other than NKIRL. For example, the NK-1 receptor-binding moiety may be a sulfur pentafluoride-containing small molecule (“N IRL-SF5”) according to one of the following structures:

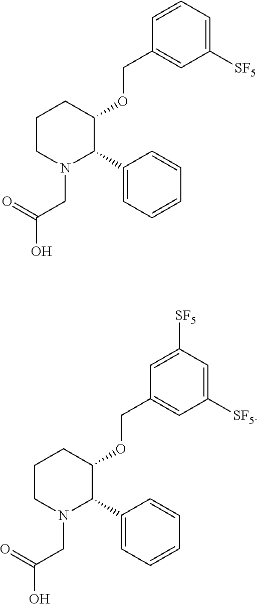

In some embodiments, the NK-1R ligand is conjugated to fluorescein, or a fluorescein derivative. In some embodiments, the NK-1R ligated is conjugated to the fluorescein derivative FITC.

The “targeting moiety” linked to the small molecule ligand binds to the recognition region of the genetically engineered CAR expressed by the CAR T cells. Accordingly, the recognition region of the CAR (e.g., a single chain fragment variable region (scFv) of an antibody, an Fab, Fv, Fc, or (Fab′)2 fragment, and the like) is directed to the “targeting moiety.” Thus, the small molecule ligand linked to a targeting moiety by a linker acts as a bridge between the cancer and the CAR T cells directing the CAR T cells to the cancer for amelioration of the cancer. In various embodiments, the bridge between the cancer and the CAR T cells can be any of the applicable conjugates shown in the Examples.

The bridge is a small organic molecule so clearance from the bloodstream can be rapidly achieved (e.g., about 20 minutes or less). In one aspect, the CAR T cell response can be targeted to only those cancer cells expressing a receptor for the small molecule ligand portion of the ‘bridge,’ thereby reducing off-target toxicity to normal tissues. Additionally, this system can be ‘universal’ because one type of CAR T cell construct can be used to target a wide variety of cancers using different ‘bridges’. Illustratively, the targeting moiety recognized by the CAR T cells may remain constant so that one type of CAR T cell construct can be used, while the small molecule ligand that binds to the cancer can be altered to allow targeting of a wide variety of cancers.

In one embodiment, a method of treatment or inhibition of a cancer is provided. The method comprises i) administering to a patient at least one dose of a CAR T cell composition comprising CAR T cells wherein the CAR T cells comprise a CAR directed to a targeting moiety; ii) administering to the patient a compound, or a pharmaceutically acceptable salt thereof, wherein the compound comprises a small molecule ligand linked to a targeting moiety by a linker and wherein the compound, or the pharmaceutically acceptable salt thereof, is administered in at least a first dose escalation sequence. In one embodiment, the compound, or the pharmaceutically acceptable salt thereof, is administered in a second dose escalation sequence.

In another embodiment, a method of treatment or inhibition of a cancer is provided. The method comprises i) administering to a patient at least one dose of a CAR T cell composition comprising CAR T cells wherein the CAR T cells comprise a CAR directed to a targeting moiety; ii) administering to the patient a compound, or a pharmaceutically acceptable salt thereof, wherein the compound comprises a small molecule ligand linked to a targeting moiety by a linker and wherein the compound, or the pharmaceutically acceptable salt thereof, is administered in a first dose escalation sequence wherein, if serious CRS occurs in the first dose escalation sequence, the compound, or the pharmaceutically acceptable salt thereof, is administered using a lower dose escalation sequence wherein the first dose of the compound, or the pharmaceutically acceptable salt thereof, in the lower dose escalation sequence is lower than the first dose of the compound, or the pharmaceutically acceptable salt thereof, administered in the first dose escalation sequence. In another embodiment, in the lower dose escalation sequence, the compound, or the pharmaceutically acceptable salt thereof, can be administered at about 0.5 percent, about 5 percent, and about 50 percent of a full dose of the compound, or the pharmaceutically acceptable salt thereof, on three separate days.

In various embodiments described in the clause list below and in the claims and throughout the application, the small molecule ligand linked to a targeting moiety by a linker is referred to as a “compound.”

### Certain Definitions

As used herein, “a” or “an” may mean one or more. As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

As used herein, the terms “treat,” “treating,” “treated,” or “treatment” refer to both therapeutic treatment and prophylactic or preventative treatment.

As used herein, the terms “ameliorate,” “ameliorating,” “amelioration,” or “ameliorated” in reference to cancer can mean reducing the symptoms of the cancer, reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic, prophylactic, or preventative treatment of the cancer.

As used herein, the terms “administer,” administering,” or “administered” mean all means of introducing the compound, or pharmaceutically acceptable salt thereof, or CAR T cell composition described herein to the patient, including, but not limited to, oral, intravenous, intramuscular, subcutaneous, and transdermal.

As used herein, the term “off-target toxicity” means organ damage or a reduction in the patient's weight that is unacceptable to the physician treating the patient, or any other effect on the patient that is unacceptable to the physician treating the patient, for example, B cell aplasia, a fever, a drop in blood pressure, or pulmonary edema.

As used herein, the terms “transduction” and “transfection” are used equivalently and the terms mean introducing a nucleic acid into a cell by any artificial method, including viral and non-viral methods.

As used herein, the term “fluorescein” can refer to the standard xanthene dye compound, as well as any fluorescein derivative. It will therefore be understood that in some embodiments, an anti-fluorescein CAR may be an anti-fluorescein derivative CAR. In one embodiment, the CAR is anti-FITC. In one embodiment, the CAR is able to bind to a FITC-folate conjugate (EC17).

As used herein, the term “stable dosing cycle” means that a therapeutically effective compound, or a pharmaceutically acceptable salt thereof, is administered at a set dosing interval for a duration of time.

As used herein, the term “dose escalation sequence” means that increasing doses of the compound, or the pharmaceutically acceptable salt thereof, are administered over time. As used herein, references to a “second dose escalation sequence”, a “third dose escalation sequence”, a “fourth dose escalation sequence”, a “fifth dose escalation sequence”, and a “sixth dose escalation sequence”, etc. mean that multiple dose escalation sequences occur, and that for each separate dose escalation sequence, after the first dose escalation sequence, the first dose of the compound, or the pharmaceutically acceptable salt thereof, in the subsequent dose escalation sequence, is lower than the last dose of the compound, or the pharmaceutically acceptable salt thereof, in the prior dose escalation sequence (see, for example, FIG. 1 and the explanation of FIG. 1 in the Brief Description of the Drawings).

Several embodiments are described by the following enumerated clauses. Any of the following embodiments in combination with any applicable embodiments described in the Summary section of this patent application, in the Detailed Description section, the Examples section, or the claims of this patent application, are also contemplated.

1. A method of treatment or inhibition of a cancer, the method comprising
i) administering to a patient at least one dose of a CAR T cell composition comprising CAR T cells wherein the CAR T cells comprise a CAR directed to a targeting moiety; and
ii) administering to the patient a compound, or a pharmaceutically acceptable salt thereof, wherein the compound comprises a small molecule ligand linked to a targeting moiety by a linker and wherein the compound, or the pharmaceutically acceptable salt thereof, is administered in at least a first dose escalation sequence. In one embodiment, the compound, or the pharmaceutically acceptable salt thereof, is administered in at least a second dose escalation sequence.

2. The method of clause 1 wherein the compound, or the pharmaceutically acceptable salt thereof, is administered in at least a first dose escalation sequence, a second dose escalation sequence, and a third dose escalation sequence.

3. The method of clause 1 wherein the compound, or the pharmaceutically acceptable salt thereof, is administered in at least a first dose escalation sequence, a second dose escalation sequence, a third dose escalation sequence, and a fourth dose escalation sequence.

4. The method of clause 1 wherein the compound, or the pharmaceutically acceptable salt thereof, is administered is administered in at least a first dose escalation sequence, a second dose escalation sequence, a third dose escalation sequence, a fourth dose escalation sequence, and a fifth dose escalation sequence.

5. The method of clause 1 wherein the compound, or the pharmaceutically acceptable salt thereof, is administered in at least a first dose escalation sequence, a second dose escalation sequence, a third dose escalation sequence, a fourth dose escalation sequence, a fifth dose escalation sequence, and a sixth dose escalation sequence.

6. The method of any one of clauses 1 to 5 wherein a first dose of the CAR T cells and a second dose of the CAR T cells are administered to the patient.

7. The method of clause 6 wherein the first dose of the CAR T cells is a test dose to monitor the patient for tolerability to the CAR T cells.

8. The method of clause 6 wherein the second dose of the CAR T cells comprises a higher dose of the CAR T cells than the first dose of the CAR T cells.

9. The method of any one of clauses 6 to 8 wherein the first dose of the CAR T cells comprises about 0.5×10⁵ of the CAR T cells per kg of patient body weight to about 1.5×10⁶ of the CAR T cells per kg of patient body weight.

10. The method of any one of clauses 6 to 9 wherein the second dose of the CAR T cells comprises about 0.8×10⁶ of the CAR T cells per kg of patient body weight to about 2×10⁷ of the CAR T cells per kg of patient body weight.

11. The method of any one of clauses 1 to 10 wherein the first dose escalation sequence is followed by a period of time during which the compound, or the pharmaceutically acceptable salt thereof, is not administered.

12. The method of any one of clauses 1 to 11 wherein the second dose escalation sequence is followed by a period of time during which the compound, or the pharmaceutically acceptable salt thereof, is not administered.

13. The method of any one of clauses 2 to 12 wherein the third dose escalation sequence is followed by a period of time during which the compound, or the pharmaceutically acceptable salt thereof, is not administered.

14. The method of any one of clauses 3 to 13 wherein the fourth dose escalation sequence is followed by a period of time during which the compound, or the pharmaceutically acceptable salt thereof, is not administered.

15. The method of any one of clauses 4 to 14 wherein the fifth dose escalation sequence is followed by a period of time during which the compound, or the pharmaceutically acceptable salt thereof, is not administered.

16. The method of any one of clauses 5 to 15 wherein the sixth dose escalation sequence is followed by a period of time during which the compound, or the pharmaceutically acceptable salt thereof, is not administered.

17. The method of any one of clauses 11 to 16 wherein the period of time is about 7 days.

18. The method of any one of clauses 1 to 17 wherein the first dose escalation sequence comprises administering to the patient about 1 percent, about 10 percent, and about 100 percent of a full dose of the compound, or the pharmaceutically acceptable salt thereof, on three separate days.

19. The method of any one of clauses 1 to 18 wherein the second dose escalation sequence comprises administering to the patient about 1 percent, about 30 percent, and about 300 percent of a full dose of the compound, or the pharmaceutically acceptable salt thereof, on three separate days.

20. The method of any one of clauses 2 to 19 wherein the third dose escalation sequence comprises administering to the patient about 1 percent, about 50 percent, and about 500 percent of a full dose of the compound, or the pharmaceutically acceptable salt thereof, on three separate days.

21. The method of any one of clauses 3 to 20 wherein the fourth dose escalation sequence comprises administering to the patient about 1 percent, about 10 percent, and about 100 percent of a full dose of the compound, or the pharmaceutically acceptable salt thereof, on three separate days.

22. The method of any one of clauses 4 to 21 wherein the fifth dose escalation sequence comprises administering to the patient about 1 percent, about 30 percent, and about 300 percent of a full dose of the compound, or the pharmaceutically acceptable salt thereof, on three separate days.

23. The method of any one of clauses 5 to 22 wherein the sixth dose escalation sequence comprises administering to the patient about 1 percent, about 50 percent, and about 500 percent of a full dose of the compound, or the pharmaceutically acceptable salt thereof, on three separate days.

24. The method of any one of clauses 18 to 23 wherein the full dose of the compound, or the pharmaceutically acceptable salt thereof, is about 10 μg/kg to about 50 μg/kg of the compound, or the pharmaceutically acceptable salt thereof.

25. The method of any one of clauses 18 to 24 wherein the full dose of the compound, or the pharmaceutically acceptable salt thereof, is about 20 μg/kg to about 40 μg/kg of the compound, or the pharmaceutically acceptable salt thereof.

26. The method of any one of clauses 18 to 25 wherein the full dose of the compound, or the pharmaceutically acceptable salt thereof, is about 25 μg/kg to about 35 μg/kg of the compound, or the pharmaceutically acceptable salt thereof.

27. The method of any one of clauses 18 to 26 wherein the full dose of the compound, or the pharmaceutically acceptable salt thereof, is about 30 μg/kg of the compound, or the pharmaceutically acceptable salt thereof.

28. The method of any one of clauses 6 to 27 wherein the first dose of the CAR T cells and the second dose of the CAR T cells are administered to the patient during week 1.

29. The method of any one of clauses 6 to 28 wherein the first dose of the CAR T cells and the second dose of the CAR T cells are administered to the patient during week 1 on Monday and Thursday.

30. The method of any one of clauses 1 to 29 wherein the first dose escalation sequence for the compound, or the pharmaceutically acceptable salt thereof, occurs during weeks 2 and 3.

31. The method of any one of clauses 1 to 30 wherein the second dose escalation sequence for the compound, or the pharmaceutically acceptable salt thereof, occurs during weeks 4 and 5.

32. The method of any one of clauses 2 to 31 wherein the third dose escalation sequence for the compound, or the pharmaceutically acceptable salt thereof, occurs during weeks 6 and 7.

33. The method of clause 30 wherein the compound, or the pharmaceutically acceptable salt thereof, is administered on three separate days and the three separate days are Monday and Thursday of week 2 and Monday of week 3.

34. The method of clause 31 wherein the compound, or the pharmaceutically acceptable salt thereof, is administered on three separate days and the three separate days are Monday and Thursday of week 4 and Monday of week 5.

35. The method of clause 32 wherein the compound, or the pharmaceutically acceptable salt thereof, is administered on three separate days and the three separate days are Monday and Thursday of week 6 and Monday of week 7.

36. The method of any one of clauses 1 to 35 wherein lymphocytes are depleted in the patient before administration of the CAR T cell composition to the patient.

37. The method of any one of clauses 1 to 36 further comprising administering platelets to the patient, administering packed red blood cells to the patient, administering cryoprecipitate to the patient, administering intravenous immunoglobulin to the patient, and/or providing antimicrobial therapy to the patient.

38. The method of any one of clauses 1 to 37 wherein, if no CRS or neurotoxicity is observed in the patient during the first dose escalation sequence, the method is advanced to the second dose escalation sequence.

39. The method of any one of clauses 2 to 38 wherein, if no CRS or neurotoxicity is observed in the patient during the second dose escalation sequence, the method is advanced to the third dose escalation sequence.

40. The method of any one of clauses 3 to 39 wherein, if no CRS or neurotoxicity is observed in the patient during the third dose escalation sequence, the method is advanced to the fourth dose escalation sequence.

41. The method of any one of clauses 4 to 40 wherein, if no CRS or neurotoxicity is observed in the patient during the fourth dose escalation sequence, the method is advanced to the fifth dose escalation sequence.

42. The method of any one of clauses 5 to 41 wherein, if no CRS or neurotoxicity is observed in the patient during the fifth dose escalation sequence, the method is advanced to the sixth dose escalation sequence.

43. The method of any one of clauses 1 to 42 wherein if fever without hypotension is observed in the patient and no neurotoxicity is observed in the patient during any one of the dose escalation sequences, all subsequent doses of the compound, or the pharmaceutically acceptable salt thereof, are administered to the patient at the dose escalation sequence level that caused the fever without hypotension.

44. The method of any one of clauses 1 to 43 wherein, if serious CRS or neurotoxicity occurs in the patient in any dose escalation sequence, all subsequent doses of the compound, or the pharmaceutically acceptable salt thereof, are administered to the patient at the dose escalation sequence level below the dose escalation sequence level that caused the serious CRS or neurotoxicity in the patient.

45. A method of treatment or inhibition of a cancer, the method comprising
i) administering to a patient at least one dose of a CAR T cell composition comprising CAR T cells wherein the CAR T cells comprise a CAR directed to a targeting moiety;
ii) administering to the patient a compound, or a pharmaceutically acceptable salt thereof, wherein the compound comprises a small molecule ligand linked to a targeting moiety by a linker and wherein the compound, or the pharmaceutically acceptable salt thereof, is administered in a first dose escalation sequence wherein, if serious CRS occurs in the first dose escalation sequence, the compound, or the pharmaceutically acceptable salt thereof, is administered using a lower dose escalation sequence wherein the first dose of the compound, or the pharmaceutically acceptable salt thereof, in the lower dose escalation sequence is lower than the first dose of the compound, or the pharmaceutically acceptable salt thereof, administered in the first dose escalation sequence.

46. The method of clause 45 wherein in the lower dose escalation sequence, the compound, or the pharmaceutically acceptable salt thereof, is administered at about 0.5 percent, about 5 percent, and about 50 percent of a full dose of the compound, or the pharmaceutically acceptable salt thereof, on three separate days.

47. The method of clause 46 wherein the full dose of the compound, or the pharmaceutically acceptable salt thereof, is about the full dose of the compound, or the pharmaceutically acceptable salt thereof, is about 10 μg/kg to about 50 μg/kg of the compound, or the pharmaceutically acceptable salt thereof.

48. The method of any one of clauses 46 to 47 wherein the full dose of the compound, or the pharmaceutically acceptable salt thereof, is about 25 μg/kg to about 35 μg/kg of the compound, or the pharmaceutically acceptable salt thereof.

49. The method of any one of clauses 46 to 48 wherein the full dose of the compound, or the pharmaceutically acceptable salt thereof, is about 30 μg/kg of the compound, or the pharmaceutically acceptable salt thereof.

50. The method of any one of clauses 1 to 49 wherein the ligand is selected from the group consisting of a folate, DUPA, an NK-1R ligand, a CAIX ligand, a ligand of gamma glutamyl transpeptidase, an NKG2D ligand, and a CCK2R ligand.

51. The method of any one of clauses 1 to 50 wherein the ligand is a folate.

52. The method of any one of clauses 1 to 50 wherein the ligand is an NK-1R ligand.

53. The method of any one of clauses 1 to 50 wherein the ligand is DUPA.

54. The method of any one of clauses 1 to 50 wherein the ligand is a CCK2R ligand.

55. The method of any one of clauses 1 to 50 wherein the ligand is a ligand of gamma glutamyl transpeptidase.

56. The method of any one of clauses 1 to 55 wherein the targeting moiety is selected from the group consisting of 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester, tetrafluorophenyl ester, a knottin, a centyrin, and a DARPin.

57. The method of any one of clauses 1 to 56 wherein the targeting moiety is FITC.

58. The method of any one of clauses 1 to 56 wherein the targeting moiety is DNP.

59. The method of any one of clauses 1 to 56 wherein the targeting moiety is TNP.

60. The method of any one of clauses 1 to 59 wherein the linker comprises polyethylene glycol (PEG), polyproline, a hydrophilic amino acid, a sugar, an unnatural peptidoglycan, a polyvinylpyrrolidone, pluronic F-127, or a combination thereof.

61. The method of any one of clauses 1 to 60 wherein the linker comprises PEG.

62. The method of any one of clauses 1 to 61 wherein the compound, or the pharmaceutically acceptable salt thereof, has the formula

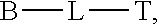

wherein B represents the small molecule ligand, L represents the linker, and T represents the targeting moiety, and wherein L comprises a structure having the formula

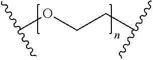

wherein n is an integer from 0 to 200.

63. The method of clause 62 wherein n is an integer from 0 to 150.

64. The method of clause 62 wherein n is an integer from 0 to 110.

65. The method of clause 62 wherein n is an integer from 0 to 20.

66. The method of clause 62 wherein n is an integer from 15 to 20.

67. The method of clause 62 wherein n is an integer from 15 to 110

68. The method of any one of clauses 1 to 67 wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, osteosarcoma, including pediatric or non-pediatric osteosarcoma, cancer of the urethra, prostate cancer, chronic leukemia, acute leukemia, acute myelocytic leukemia, lymphocytic lymphoma, myeloid leukemia, myelomonocytic leukemia, hairy cell leukemia, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, and adenocarcinoma of the gastroesophageal junction.

69. The method of any one of clauses 1 to 51 or 56 to 68 wherein the cancer is a folate receptor expressing cancer.

70. The method of any one of clauses 1 to 69 wherein the cancer is an osteosarcoma.

71. The method of any one of clauses 1 to 70 wherein the CAR has a recognition region and the recognition region is a single chain fragment variable (scFv) region of an antibody.

72. The method of any one of clauses 1 to 57 or 60 to 71 wherein the CAR has a recognition region and the recognition region of the CAR is a single chain fragment variable (scFv) region of an anti-FITC antibody.

73. The method of any one of clauses 1 to 72 wherein the CAR has a co-stimulation domain and the co-stimulation domain is selected from the group consisting of CD28, CD137 (4-1 BB), CD134 (0X40), and CD278 (ICOS).

74. The method of any one of clauses 1 to 73 wherein the CAR has an activation signaling domain and the activation signaling domain is a T cell CD3ζ chain or an Fc receptor γ.

75. The method of any one of clauses 1 to 57 or 60 to 74 wherein the CAR has a recognition region and the recognition region is a single chain fragment variable (scFv) region of an anti-FITC antibody, wherein the CAR has a co-stimulation domain and the co-stimulation domain is CD137 (4-1BB), and wherein the CAR has an activation signaling domain and the activation signaling domain is a T cell CD3ζ chain.

76. The method of any one of clauses 1 to 75 wherein the patient is imaged prior to administration of the compound, or the pharmaceutically acceptable salt thereof, or prior to administration of the CAR T cell composition.

77. The method of any one of clauses 1 to 76 wherein the compound, or the pharmaceutically acceptable salt thereof, is not an antibody, and does not comprise a fragment of an antibody.

78. The method of any one of clauses 1 to 77 wherein the targeting moiety does not comprise a peptide epitope.

79. The method of any one of clauses 1 to 78 wherein cytokine release resulting in off-target toxicity in the patient does not occur and wherein CAR T cell toxicity to the cancer occurs.

80. The method of any one of clauses 1 to 78 wherein off-target tissue toxicity does not occur in the patient and wherein CAR T cell toxicity to the cancer occurs.

81. The method of any one of clauses 1 to 78 wherein the cancer comprises a tumor, wherein tumor size is reduced in the patient, and wherein off-target toxicity does not occur.

82. The method of any one of clauses 1 to 81 wherein the CAR T cells comprise a nucleic acid comprising SEQ ID NO:01.

83. The method of any one of clauses 1 to 82 wherein the CAR T cells comprise a polypeptide comprising SEQ ID NO:02.

84. The method of clause 82 wherein the nucleic acid encodes a chimeric antigen receptor.

85. The method of any one of clauses 1 to 81 wherein the CAR T cells comprise a nucleic acid comprising SEQ ID NO:04.

86. The method of any one of clauses 1 to 81 or 85 wherein the CAR T cells comprise a polypeptide comprising SEQ ID NO:05.

87. The method of clause 85 wherein the nucleic acid encodes a chimeric antigen receptor.

88. The method of any one of clauses 1 to 87 wherein the CAR comprises humanized amino acid sequences.

89. The method of any one of clauses 1 to 87 wherein the CAR consists of humanized amino acid sequences.

90. The method of any one of clauses 1 to 89 further comprising administering to the patient a folate, a conjugate comprising a folate wherein the conjugate comprising a folate does not comprise a targeting moiety, or an agent that inhibits activation of the CAR T cells.

91. The method of clause 90 wherein a folate is administered.

92. The method of clause 90 wherein folic acid or leucovorin is administered.

93. The method of clause 90 wherein the conjugate comprising a folate is administered.

94. The method of clause 93 wherein the conjugate comprising a folate comprises a folate linked to one or more amino acids.

95. The method of clause 93 wherein the conjugate comprising a folate has the formula

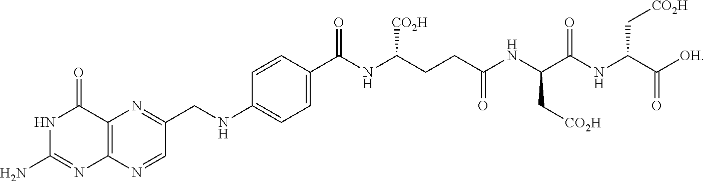 [SMILES: Nc1nc2ncc(CNc3ccc(C(=O)N[C@@H](CCC(=O)N[C@H](CC(=O)O)C(=O)N[C@H](CC(=O)O)C(=O)O)C(=O)O)cc3)nc2c(=O)[nH]1]

96. The method of clause 91 wherein the folate has the formula

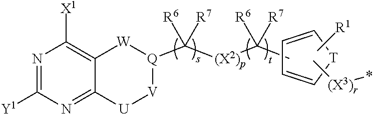

wherein X¹ and Y¹ are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents an optional covalent bond to the rest of the conjugate, if any additional chemical moieties are part of the folate.

97. The method of clause 90 wherein the agent that inhibits activation of the CAR T cells is selected from the group consisting of a lymphocyte-specific

protein tyrosine kinase inhibitor, a PI3 kinase inhibitor, an inhibitor of an IL-2 inducible T cell kinase, a JAK inhibitor, a BTK inhibitor, EC2319, and an agent that blocks CAR T cell binding to the compound, or the pharmaceutically acceptable salt thereof, but does not bind to the cancer.

98. The method of clause 90 wherein the agent that inhibits activation of the CAR T cells is administered and the agent is a lymphocyte-specific protein tyrosine kinase inhibitor.

99. The method of clause 98 wherein the lymphocyte-specific protein tyrosine kinase inhibitor is Dasatinib.

100. The method of clause 90 wherein the agent that inhibits activation of the CAR T cells is administered and the agent is a PI3 kinase inhibitor.

101. The method of clause 100 wherein the PI3 kinase inhibitor is GDC0980.

102. The method of clause 90 wherein the agent that inhibits activation of the CAR T cells is administered and the agent is an IL-2 inducible T cell kinase inhibitor.

103. The method of clause 102 wherein the IL-2 inducible T cell kinase inhibitor is BMS-509744.

104. The method of clause 90 wherein the agent that inhibits activation of the CAR T cells is administered and is an agent that blocks CAR T cell binding to the compound, or the pharmaceutically acceptable salt thereof, but does not bind to the cancer.

105. The method of clause 104 wherein the agent is fluoresceinamine, FITC, or sodium fluorescein.

106. The method of clause 105 wherein the agent is sodium fluorescein.

107. The method of any one of clauses 90 to 106 wherein administration of the folate, the conjugate comprising a folate wherein the conjugate comprising a folate does not comprise a targeting moiety, or the agent that inhibits activation of the CAR T cells causes reduction in cytokine levels in the patient.

108. The method of clause 107 wherein the reduction in cytokine levels is a reduction to about the cytokine levels in an untreated patient.

109. The method of any one of clauses 90 to 108 wherein the cancer comprises a tumor and tumor size in the patient is not increased when the folate, the conjugate comprising a folate wherein the conjugate comprising a folate does not comprise a targeting moiety, or the agent that inhibits activation of the CAR T cells is administered to the patient.

110. The method of any one of clauses 104 to 106 wherein the agent that inhibits activation of the CAR T cells is administered to the patient when the CRS grade reaches 1, 2, 3, or 4.

111. The method of clause 110 wherein the agent that inhibits activation of the CAR T cells is administered to the patient when the CRS grade reaches 3 or 4.

112. The method of any one of clauses 104 to 106 wherein the agent that inhibits activation of the CAR T cells is administered at a dose of about 0.01 to about 300 μmoles/kg of body weight of the patient.

113. The method of any one of clauses 1 to 112 wherein CRS is reduced or prevented in the patient and the method results in a decrease in tumor volume in the patient.

114. The method of any one of clauses 1 to 113 wherein body weight loss due to CRS is reduced or prevented.

115. The method of any one of clauses 90 to 114 further comprising re-administering the compound, or the pharmaceutically acceptable salt thereof, to the patient.

116. The method of clause 115 wherein the subsequent administration of the compound, or the pharmaceutically acceptable salt thereof, causes CAR T cell activation and an increase in cytokine levels in the patient.

117. The method of any one of clauses 1 to 116 wherein the CAR T cell composition is administered before the compound, or the pharmaceutically acceptable salt thereof.

118. The method of any one of clauses 1 to 117 wherein the CAR T cells are autologous.

119. The method of any one of clauses 2 to 17 wherein the first dose escalation sequence comprises administering to the patient about 1 percent, about 2 percent, and about 20 percent of a full dose of the compound, or the pharmaceutically acceptable salt thereof, on three separate days, wherein the second dose escalation sequence comprises administering to the patient about 1 percent, about 6 percent, and about 60 percent of a full dose of the compound, or the pharmaceutically acceptable salt thereof, on three separate days, and wherein the third dose escalation sequence comprises administering to the patient about 1 percent, about 10 percent, and about 100 percent of a full dose of the compound, or the pharmaceutically acceptable salt thereof, on three separate days.

120. The method of any one of clauses 2 to 17 wherein the first dose escalation sequence comprises administering to the patient about 1 percent, about 10 percent, and about 100 percent of a full dose of the compound, or the pharmaceutically acceptable salt thereof, on three separate days, and wherein the second dose escalation sequence comprises administering to the patient about 1 percent, about 20 percent, and about 200 percent of a full dose of the compound, or the pharmaceutically acceptable salt thereof, on three separate days.

121. The method of any one of clauses 2 to 17 wherein the first dose escalation sequence comprises administering to the patient about 1 percent, about 10 percent, and about 100 percent of a full dose of the compound, or the pharmaceutically acceptable salt thereof, on three separate days, and wherein the second dose escalation sequence comprises administering to the patient about 1 percent, about 10 percent, and about 100 percent of a full dose of the compound, or the pharmaceutically acceptable salt thereof, on three separate days.

122. The method of any one of clauses 2 to 17 wherein the first dose escalation sequence comprises administering to the patient about 1 percent, about 20 percent, and about 200 percent of a full dose of the compound, or the pharmaceutically acceptable salt thereof, on three separate days, and wherein the second dose escalation sequence comprises administering to the patient about 1 percent, about 20 percent, and about 200 percent of a full dose of the compound, or the pharmaceutically acceptable salt thereof, on three separate days.

123. The method of any one of clauses 119 to 122 wherein the full dose of the compound, or the pharmaceutically acceptable salt thereof, is about 500 nmoles/kg.

Accordingly, various embodiments are provided in the preceding paragraphs and in the clause list above, and all applicable embodiments described in this “Detailed Description” the “Summary” section, the Examples, and the claims apply to these embodiments.

As described herein, a “patient” or “subject” can be a human or, in the case of veterinary applications, the patient or subject can be a laboratory, an agricultural, a domestic, or a wild animal. In various aspects, the patient or subject can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, a goat, or a wild animal in captivity such as a bear, a panda, a lion, a tiger, a leopard, an elephant, a zebra, a giraffe, a gorilla, a dolphin, or a whale.

In various embodiments, the cancer to be treated or inhibited can be selected from a carcinoma, a sarcoma, a lymphoma, a melanoma, a mesothelioma, a nasopharyngeal carcinoma, a leukemia, an adenocarcinoma, or a myeloma. In other embodiments, the cancer may be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, osteosarcoma, including pediatric or non-pediatric osteosarcoma, cancer of the urethra, prostate cancer, chronic leukemia, acute leukemia, including acute myelocytic leukemia, a lymphocytic lymphoma, myeloid leukemia, myelomonocytic leukemia, hairy cell leukemia, pleural mesothelioma, cancer of the bladder, Burkitt's lymphoma, cancer of the ureter, cancer of the kidney, renal cell carcinoma, carcinoma of the renal pelvis, a neoplasm of the central nervous system (CNS), primary CNS lymphoma, a spinal axis tumor, a brain stem glioma, a pituitary adenoma, or an adenocarcinoma of the gastroesophageal junction.

In some aspects of these embodiments, the cancer is a folate receptor expressing cancer. In another embodiment, the cancer is a folate receptor a-expressing cancer. In yet another embodiment, the cancer is a folate receptor b-expressing cancer. In some aspects of these embodiments, the cancer is an endometrial cancer, a non-small cell lung cancer, an ovarian cancer, an osteosarcoma, including pediatric or non-pediatric osteosarcoma, or a triple-negative breast cancer. In another embodiment, the cancer being treated or inhibited is a tumor. In another embodiment, the cancer is malignant. In another embodiment, the cancer is an osteosarcoma including pediatric or non-pediatric osteosarcoma.

In one embodiment, the “small molecule ligand” can be a folate, DUPA (a ligand bound by PSMA-positive human prostate cancer cells and other cancer cell types), an NK-1R ligand (receptors for the NK-1R ligand are found, for example, on cancers of the colon and pancreas), a CAIX ligand (receptors for the CAIX ligand are found, for example, on renal, ovarian, vulvar, and breast cancers), a ligand of gamma glutamyl transpeptidase (the transpeptidase is overexpressed, for example, in ovarian cancer, colon cancer, liver cancer, astrocytic gliomas, melanomas, and leukemias), an NKG2D ligand (receptors for the NKG2D ligand are found, for example, on cancers of the lung, colon, kidney, prostate, and on T and B cell lymphomas), or a CCK2R ligand (receptors for the CCK2R ligand are found on cancers of the thyroid, lung, pancreas, ovary, brain, stomach, gastrointestinal stroma, and colon, among others), each of which is a small molecule ligand that binds specifically to a cancer cell type (i.e., the receptor for each of these ligands can be overexpressed on cancers compared to normal tissues).

In one embodiment, the small molecule ligand may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one embodiment, a DUPA derivative can be the ligand of the small molecule ligand linked to a targeting moiety, and DUPA derivatives are described in WO 2015/057852, which is expressly incorporated herein by reference.

In one embodiment, the small molecule ligand in the context of the “small molecule ligand linked to a linker” is a folate. In various embodiments, the folate can be folic acid, a folic acid analog, or another folate receptor-binding molecule. In various embodiments, analogs of folate that can be used include folinic acid (e.g., leucovorin), pteropoly glutamic acid, and folate receptor-binding pteridines such as tetrahydropterins, dihydrofolates, tetrahydrofolates, or their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, or 10-deaza analogs. The dideaza analogs include, for example, 1,5 dideaza, 5,10-dideaza, 8,10-dideaza, or 5,8-dideaza analogs. The foregoing folic acid analogs are conventionally termed “folates,” reflecting their capacity to bind to folate receptors. Other folate receptor-binding analogs useful in aspect described herein include aminopterin, amethopterin (methotrexate), N10-methylfolate, 2-deamino-hydroxyfolate, deaza analogs such as 1-deazamethopterin or 3-deazamethopterin, or 3′,5′-dichloro-4-amino-4-deoxy-NlO-methylpteroylglutamic acid (dichloromethotrexate).

In another embodiment, the small molecule ligand in the context of the “small molecule ligand linked to a linker” can have the formula:

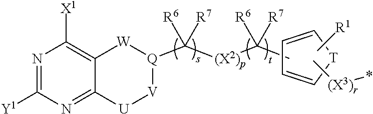

wherein X¹ and Y¹ are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents an optional covalent bond to the rest of the conjugate, if any additional chemical moieties are part of the folate.

In one aspect, the “targeting moiety” that binds to the CAR expressed by CAR T cells can be selected, for example, from 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester, tetrafluorophenyl ester, a knottin, a centyrin, a DARPin, an affibody, an affilin, an anticalin, an atrimer, an avimer, a bicicyclic peptide, an FN3 scaffold, a cys-knot, a fynomer, a Kunitz domain, or an Obody. The identity of the targeting moiety is limited only in that it should be recognized and bound by the CAR, preferably with specificity, and that it has a relatively low molecular weight. In various aspects, exemplary targeting moieties are haptens, including small molecular weight organic molecules.

In one illustrative embodiment, the targeting moiety can have the following illustrative structure:

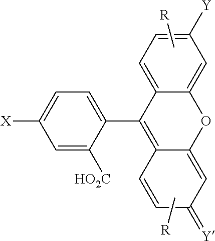

wherein X is oxygen, nitrogen, or sulfur, and where X is attached to linker L; Y is OR^{a}, NR^{a}₂, or NR^{a}₃⁺; and Y′ is O, NR^{a}, or NR^{a}₂⁺; where each R is independently selected in each instance from H, fluoro, sulfonic acid, sulfonate, and salts thereof, and the like; and R^{a} is hydrogen or alkyl.

In one illustrative aspect, the linker can comprise polyethylene glycol (PEG), polyproline, a hydrophilic amino acid, a sugar, an unnatural peptidoglycan, a polyvinylpyrrolidone, pluronic F-127, or a combination thereof.

In another illustrative aspect, the linker in the compound, or pharmaceutically acceptable salt thereof, described herein can comprise a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Daltons) are employed. In another embodiment, linker lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In various embodiments, the small molecule ligand linked to a targeting moiety can be of the formula:

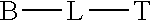

wherein B represents the small molecule ligand, L represents the linker, and T represents the targeting moiety, and wherein L comprises a structure having the formula

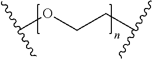

wherein n is an integer from 0 to 200. In another embodiment, n can be an integer from 0 to 150, 0 to 110, 0 to 100, 0 to 90, 0 to 80, 0 to 70, 0 to 60, 0 to 50, 0 to 40, 0 to 30, 0 to 20, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 0 to 1, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 15 to 31, 15 to 32, 15 to 33, 15 to 34, 15 to 35, 15 to 36, 15 to 37, 15 to 38, 15 to 39, 15 to 40, 15 to 50, 15 to 60, 15 to 70, 15 to 80, 15 to 90, 15 to 100, 15 to 110, 15 to 120, 15 to 130, 15 to 140, 15 to 150, or n can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 108, 110, 120, 130, 140, or 150.

In another embodiment, the linker may be a divalent linker that may include one or more spacers. Illustrative spacers are shown in the following table. The following non-limiting, illustrative spacers are described where * indicates the point of attachment to the small molecule ligand or to the targeting moiety, or to other divalent linker portions.

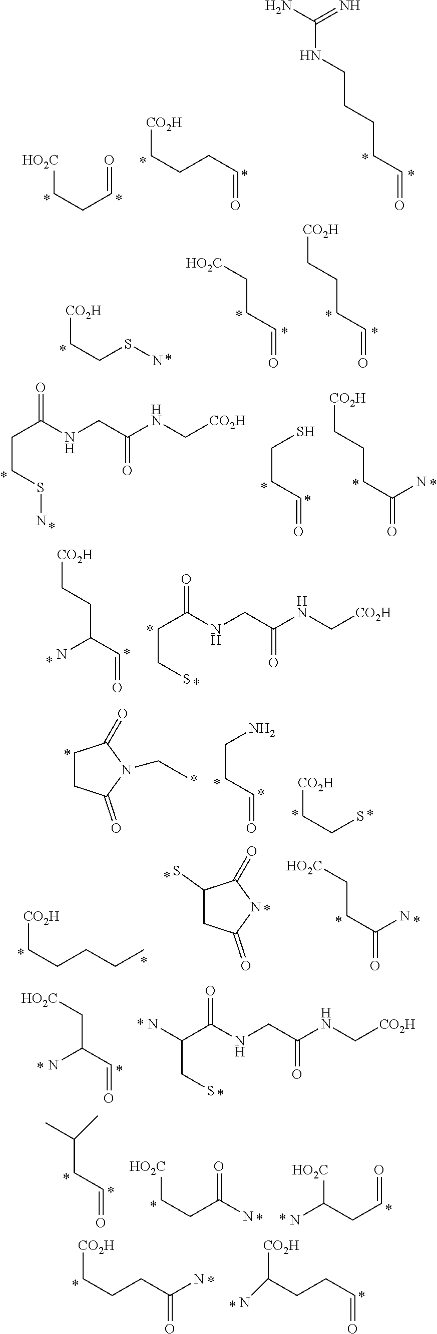

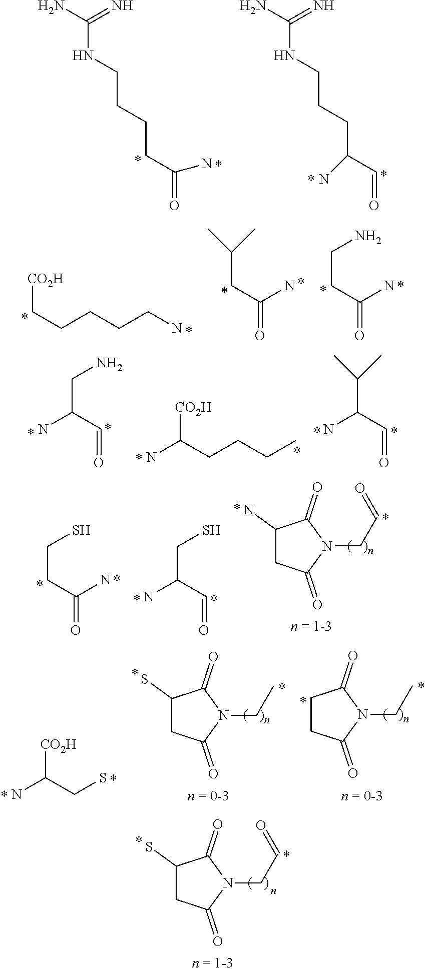

In other embodiments, the small molecule ligand linked to a targeting moiety (bridge) can have any of the following structures.

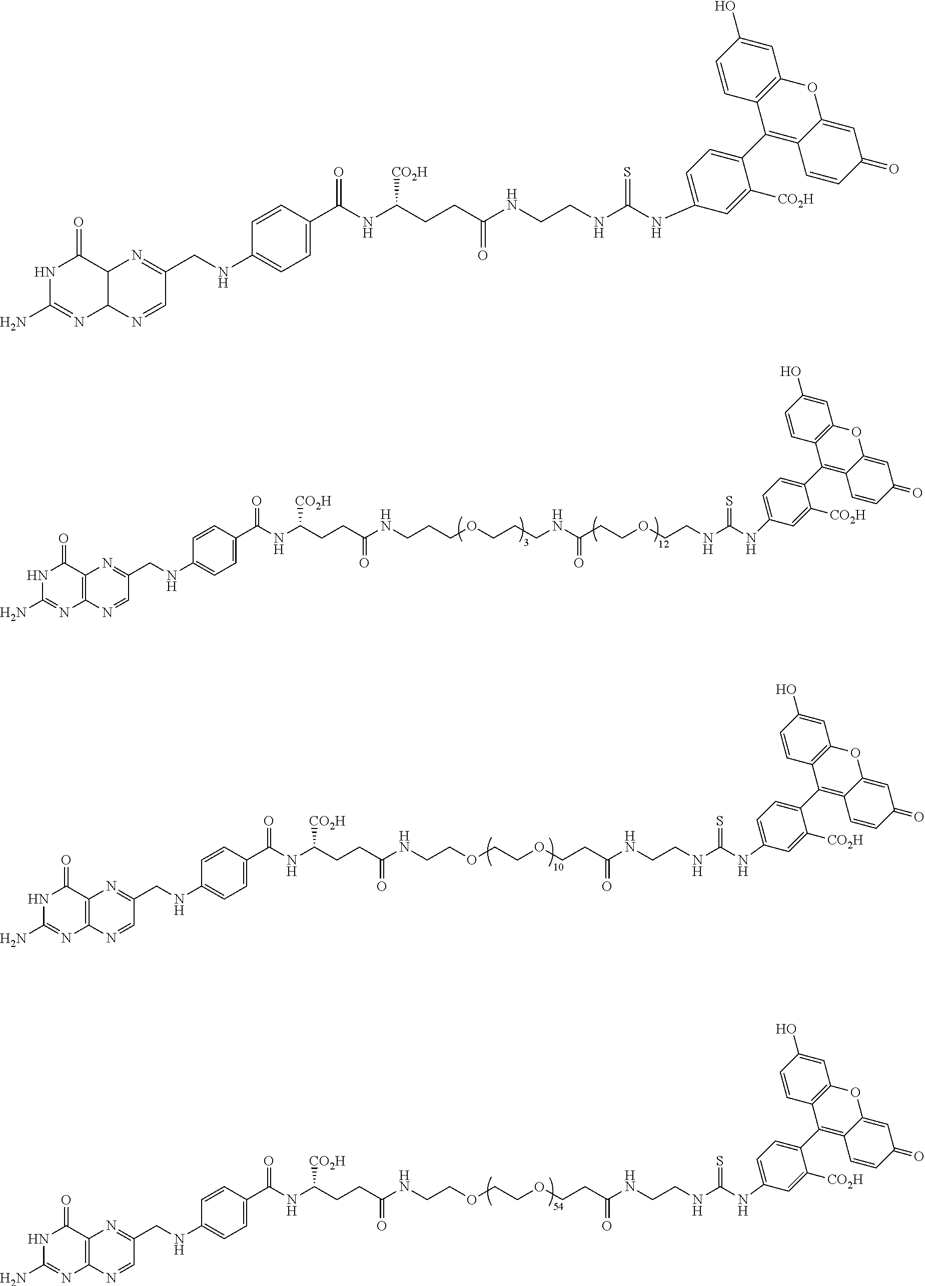

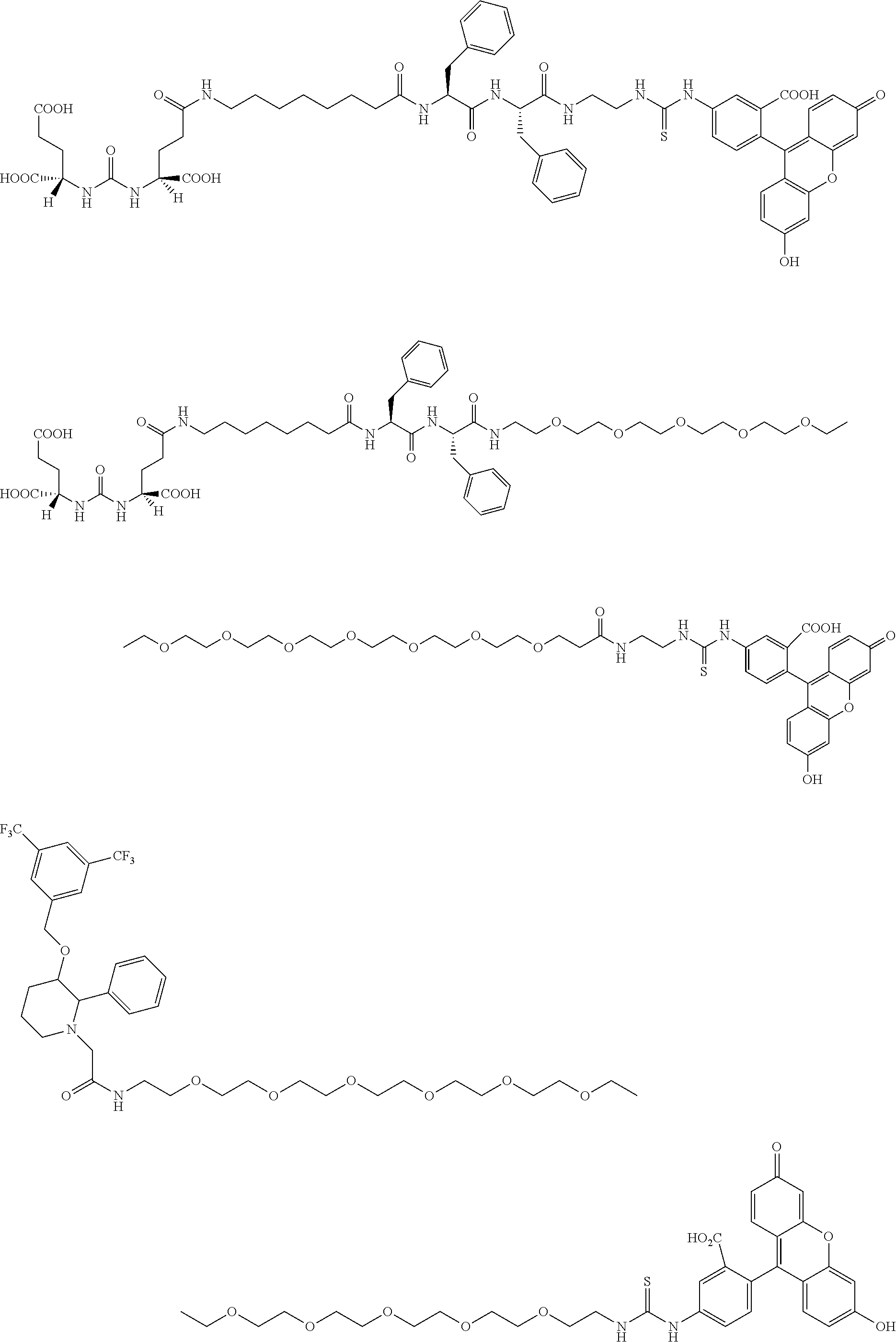

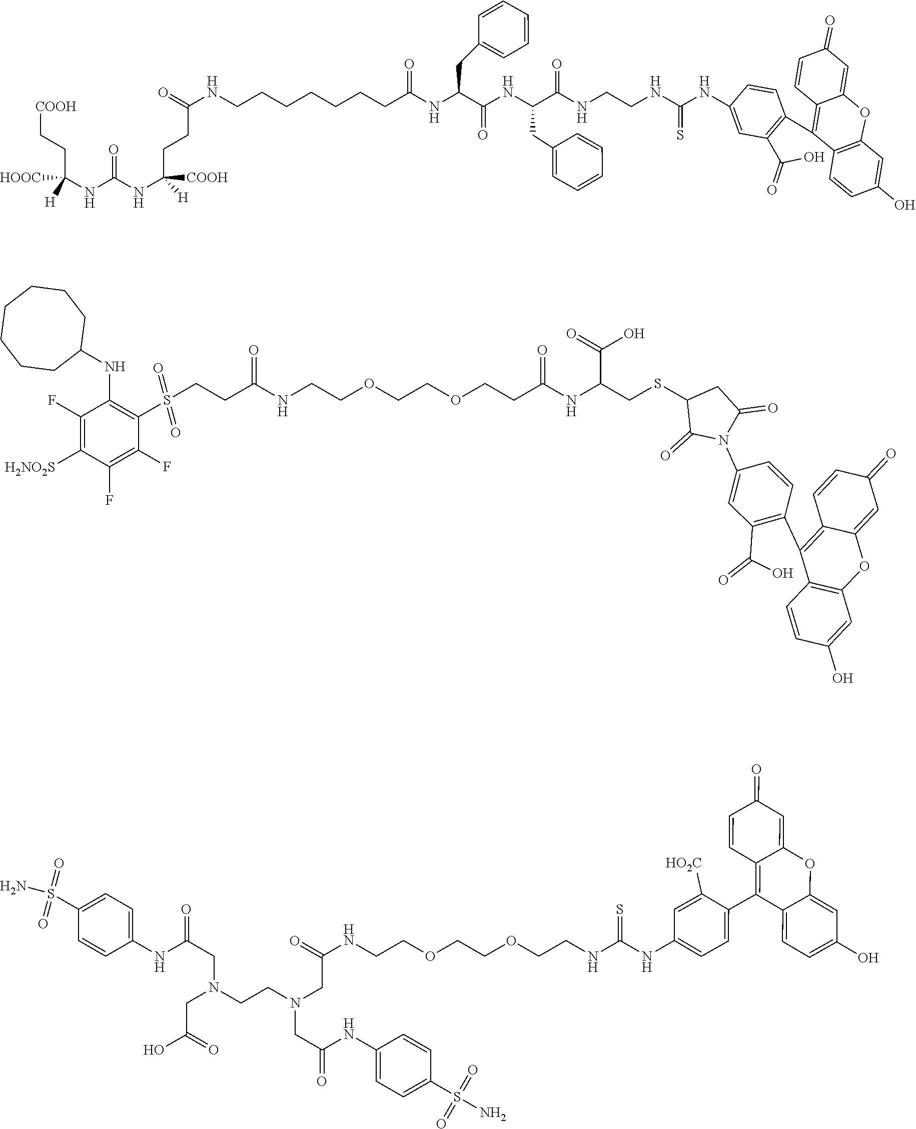 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O ; NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F ; NS(=O)(=O)c1ccc(NC(=O)CN(CCN(CC(=O)NCCOCCOCCNC(=S)Nc2ccc(-c3c4ccc(=O)cc-4oc4cc(O)ccc34)c(C(=O)O)c2)CC(=O)Nc2ccc(S(N)(=O)=O)cc2)CC(=O)O)cc1]

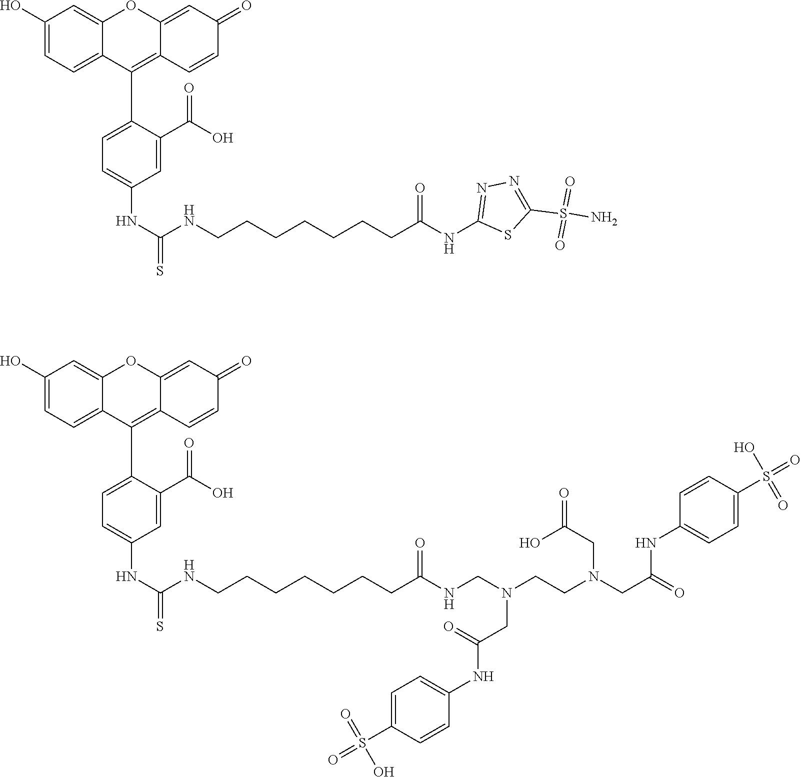 [SMILES: NS(=O)(=O)c1nnc(NC(=O)CCCCCCCNC(=S)Nc2ccc(-c3c4ccc(=O)cc-4oc4cc(O)ccc34)c(C(=O)O)c2)s1 ; O=C(O)CN(CCN(CNC(=O)CCCCCCCNC(=S)Nc1ccc(-c2c3ccc(=O)cc-3oc3cc(O)ccc23)c(C(=O)O)c1)CC(=O)Nc1ccc(S(=O)(=O)O)cc1)CC(=O)Nc1ccc(S(=O)(=O)O)cc1]

In other embodiments, the compound, or the pharmaceutically acceptable salt thereof, is not an antibody, and does not comprise a fragment of an antibody. In yet another embodiment, the targeting moiety does not comprise a peptide epitope.

In one illustrative embodiment, the small molecule ligand linked to a targeting moiety by a linker (the bridge) comprises fluorescein isothiocyanate (FITC) linked to the small molecule ligand. In one aspect, the cancer may overexpress a receptor for the small molecule ligand. In another aspect, for example, cytotoxic T cells, or another type of T cell, can be transformed to express a CAR that comprises anti-FITC scFv. In this aspect, the CAR may target FITC, wherein the cancer is decorated with FITC molecules as a result of binding of the small molecule ligand to the cancer. Thus, toxicity to normal, non-target cells can be avoided or reduced. In this embodiment, when the anti-FITC CAR-expressing T cells bind FITC, the CAR T cells are activated and the cancer is ameliorated or inhibited.

A “pharmaceutically acceptable salt” of a small molecule ligand linked to a targeting moiety by a linker is contemplated. As used herein, the term “pharmaceutically acceptable salt” refers to those salts whose counter ions may be used in pharmaceuticals. In various embodiments, such salts include, but are not limited to 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt is contemplated in connection with the embodiments described herein.

In various embodiments, suitable acid addition salts are formed from acids, which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate or trifluoroacetate salts.

In various embodiments, suitable base salts are formed from bases, which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate or hemicalcium salts.

In one illustrative aspect, the compound, or a pharmaceutically salt thereof, described herein may contain one or more chiral centers, or may otherwise be capable of existing as multiple stereoisomers. Accordingly, various embodiments may include pure stereoisomers as well as mixtures of stereoisomers, such as enantiomers, diastereomers, and enantiomerically or diastereomerically enriched mixtures. In one aspect, the compound, or pharmaceutically acceptable salt thereof, described herein may be capable of existing as geometric isomers. Accordingly, various embodiments may include pure geometric isomers or mixtures of geometric isomers.

In some aspects, the compound, or pharmaceutically acceptable salt thereof, described herein may exist in unsolvated forms as well as solvated forms, including hydrated forms. In general, the solvated forms are equivalent to unsolvated forms and are encompassed within the scope of the present invention.

The methods described herein also utilize T lymphocytes (e.g., cytotoxic T lymphocytes) engineered to express a chimeric antigen receptor (CAR) that recognizes and binds to the targeting moiety (e.g., FITC, DNP, or TNP) of the bridge. In one embodiment, the CARs described herein comprise three domains including 1) a recognition region (e.g., a single chain fragment variable (scFv) region of an antibody, a Fab fragment, and the like) which recognizes and binds to the targeting moiety with specificity, 2) a co-stimulation domain which enhances the proliferation and survival of the T lymphocytes, and 3) an activation signaling domain which generates a T lymphocyte activation signal.

In various aspects, as non-limiting examples, scFv regions of antibodies that bind 2,4-dinitrophenol (DNP), 2,4,6-trinitrophenol (TNP), biotin, digoxigenin, fluorescein, fluorescein isothiocyanate (FITC), NHS-fluorescein, pentafluorophenyl ester, tetrafluorophenyl ester, a knottin, a centyrin, a DARPin, an affibody, an affilin, an anticalin, an atrimer, an avimer, a bicicyclic peptide, an FN3 scaffold, a cys-knot, a fynomer, a Kunitz domain, or an Obody can be used. In illustrative non-limiting embodiments, the scFv regions can be prepared from (i) an antibody known in the art that binds a targeting moiety, (ii) an antibody newly prepared using a selected targeting moiety, such as a hapten, and (iii) sequence variants derived from the scFv regions of such antibodies, e.g., scFv regions having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the scFv region from which they are derived.

In one aspect, the co-stimulation domain serves to enhance the proliferation and survival of the cytotoxic T lymphocytes upon binding of the CAR to a targeting moiety.

Suitable co-stimulation domains include, but are not limited to, CD28, CD137 (4-1BB), a member of the tumor necrosis factor (TNF) receptor family, CD134 (OX40), a member of the TNFR-superfamily of receptors, CD27, CD30, CD150, DAP10, NKG2D, and CD278 (ICOS), a CD28-superfamily co-stimulatory molecule expressed on activated T cells, or combinations thereof. A skilled artisan will understand that sequence variants of these co-stimulation domains can be used without adversely impacting the invention, where the variants have the same or similar activity as the domain upon which they are modeled. In various embodiments, such variants can have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the domain from which they are derived.

In an illustrative embodiment, the activation signaling domain serves to activate T lymphocytes (e.g., cytotoxic T lymphocytes) upon binding of the CAR to a targeting moiety. In various embodiments, suitable activation signaling domains include the T cell CD3ζ chain, CD3 delta receptor protein, mb1 receptor protein, B29 receptor protein, and the Fc receptor γ. The skilled artisan will understand that sequence variants of these activation signaling domains can be used where the variants have the same or similar activity as the domain upon which they are modeled. In various embodiments, the variants have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the domain from which they are derived.

In one aspect, constructs encoding the CARs are prepared using genetic engineering techniques. Such techniques are described in detail in Sambrook et al., “Molecular Cloning: A Laboratory Manual”, 3rd Edition, Cold Spring Harbor Laboratory Press, (2001), incorporated herein by reference, and Green and Sambrook, “Molecular Cloning; A Laboratory Manual”, 4th Edition, Cold Spring Harbor Laboratory Press, (2012), incorporated herein by reference.

As examples, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein comprising a recognition region, one or more co-stimulation domains, and an activation signaling domain, in frame and linked in a 5′ to 3′ direction. In other embodiments, other arrangements are acceptable and include a recognition region, an activation signaling domain, and one or more co-stimulation domains. In one embodiment, the placement of the recognition region in the fusion protein will generally be such that display of the region on the exterior of the CAR T cell is achieved. In one embodiment, the CARs may include additional elements, such as a signal peptide (e.g., CD8α signal peptide) to ensure proper export of the fusion protein to the cell surface, a transmembrane domain to ensure the fusion protein is maintained as an integral membrane protein (e.g., CD8α transmembrane domain, CD28 transmembrane domain, or CD3ζ transmembrane domain), and a hinge domain (e.g., CD8α hinge) that imparts flexibility to the recognition region and allows strong binding to the targeting moiety.

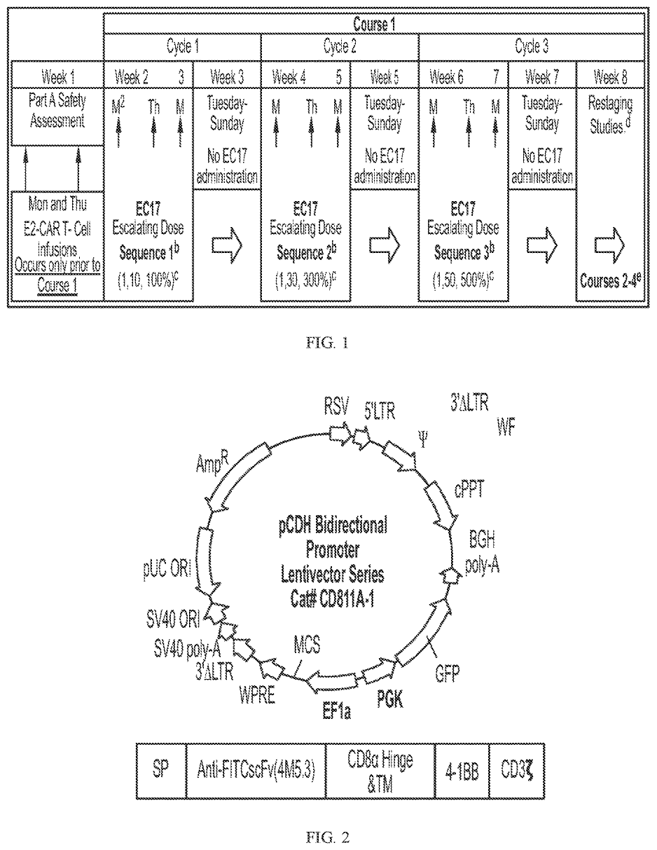

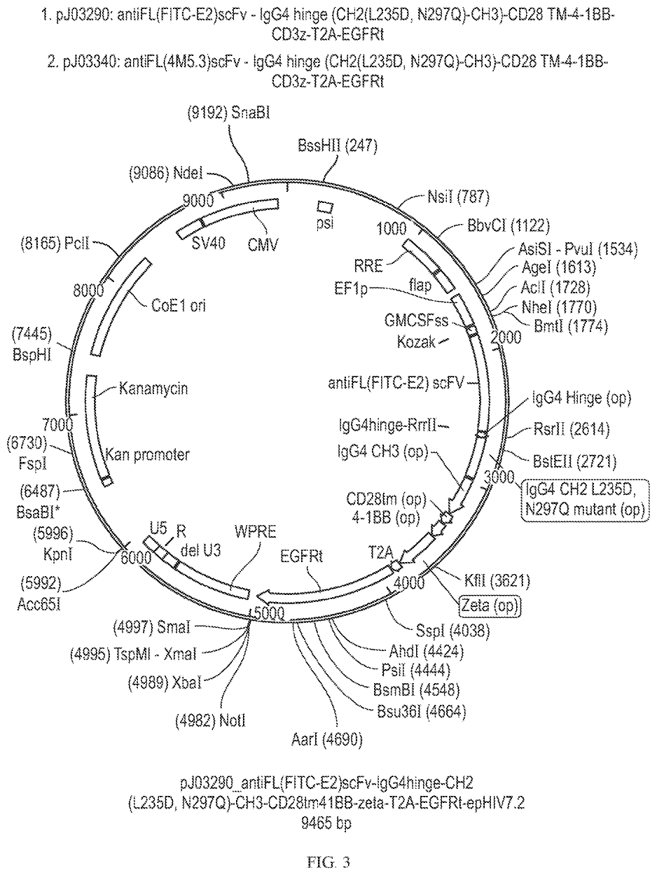

Diagrams of exemplary CARs are shown in FIG. 2 and FIG. 3. For FIG. 2, the fusion protein sequence can be incorporated into a lentivirus expression vector where “SP” is a signal peptide, the CAR is an anti-FITC CAR, a CD8α hinge and a CD8α transmembrane domain are present, the co-stimulation domain is 4-1BB, and the activation signaling domain is CD3ζ. Exemplary nucleic acid sequences of a CAR insert are provided as SEQ ID NO:01 and SEQ ID NO:03 and the encoded amino acid sequence is provided as SEQ ID NO:02. In yet another embodiment, SEQ ID NO:02 can comprise or consist of humanized, or human amino acid sequences.

For FIG. 3, a diagram of an exemplary CAR construct, wherein the expressed CAR comprises an E2 anti-fluorescein antibody fragment is shown where the fusion protein sequence can be incorporated into an expression vector and where the CAR comprises an E2 anti-fluorescein antibody fragment, an IgG4 hinge domain, a CD28 transmembrane domain, and where the co-stimulation domain is CD137 (4-1BB), and the activation signaling domain is CD3ζ. The CAR can comprise additional suitable domains. An exemplary nucleic acid sequence of such a CAR insert is provided as SEQ ID NO:04 and the exemplary encoded amino acid sequence is provided as SEQ ID NO:05. SEQ ID NO:04 includes the sequence beginning at the underlined “AGE” codon and ending with the underlined “GGC” codon (See TABLE 1). This portion of the longer sequence, encodes the CAR that is inserted into the T cell membrane. The other portions of the longer sequence include coding sequence for signal peptides, the EGFRt domain, etc. which are not part of the CAR that is inserted into the membrane and which functions as the chimeric antigen receptor. SEQ ID NO:05 includes the sequence beginning at the underlined “S” and ending with the underlined “G” (See TABLE 1). This portion of the longer sequence is the amino acid sequence for the CAR that is inserted into the T cell membrane. The other portions of the longer sequence include amino acid sequences for signal peptides, the EGFRt domain, etc. which are not part of the CAR inserted into the membrane and which functions as the chimeric antigen receptor. In yet another embodiment, SEQ ID NO:05 can comprise or consist of humanized, or human amino acid sequences. SEQ ID NO:04 and SEQ ID NO:05 are as described above and which are shown in TABLE 1. The start and stop codons in SEQ ID NO:04 (ATG, AGC, and GGC, respectively) are underlined and the longer sequence (SEQ ID NO:08) is an exemplary sequence that can be used for transduction of T cells for use in the methods as described herein.

Another exemplary CAR construct is the 4M5.3 CAR shown diagrammatically in FIG. 3. SEQ ID NO:06 includes the sequence shown below beginning at the underlined “GAC” codon and ending with the underlined “GGC” codon (See TABLE 1). This portion of the longer sequence, encodes the exemplary 4M5.3 CAR. The CAR is inserted into the T cell membrane. The other portions of the longer sequence include coding sequence for signal peptides, the EGFRt domain, etc. which are not part of the CAR that is inserted into the membrane and which functions as the chimeric antigen receptor. SEQ ID NO:07 includes the sequence beginning at the underlined “D” and ending with the underlined “G” (See TABLE 1). This portion of the longer sequence is the amino acid sequence for the CAR that is inserted into the T cell membrane. The other portions of the longer sequence include amino acid sequences for signal peptides, the EGFRt domain, etc. which are not part of the CAR inserted into the membrane and which functions as the chimeric antigen receptor. In yet another embodiment, SEQ ID NO:07 can comprise or consist of humanized, or human amino acid sequences. SEQ ID NO:06 and SEQ ID NO:07 are as described above and which are shown below. The start and stop codons in the longer nucleic acid sequence are underlined and the longer sequence is an exemplary sequence that can be used for transduction of T cells to prepare the 4M5.3 CAR.

In one embodiment, the CAR has a recognition region and the recognition region is a single chain fragment variable (scFv) region of an anti-FITC antibody, a co-stimulation domain and the co-stimulation domain is CD137 (4-1BB), and an activation signaling domain and the activation signaling domain is a T cell CD3ζ chain. It is well-known to the skilled artisan that an anti-FITC scFv and an anti-fluorescein scFv are equivalent terms.

In one embodiment, T lymphocytes (e.g., cytotoxic T lymphocytes) can be genetically engineered to express CAR constructs by transfecting a population of the T lymphocytes with an expression vector encoding the CAR construct. Suitable methods for preparing a transduced population of T lymphocytes expressing a selected CAR construct are well-known to the skilled artisan, and are described in Sambrook et et, “Molecular Cloning: A Laboratory Manual”, 3rd Edition, Cold Spring Harbor Laboratory Press, (2001), incorporated herein by reference, and Green and Sambrook, “Molecular Cloning: A Laboratory Manual”, 4th Edition, Cold Spring Harbor Laboratory Press, (2012), incorporated herein by reference.

In one embodiment, CAR T cells comprising a nucleic acid of SEQ ID NO:01, SEQ ID NO:03, SEQ ID NO:04, or SEQ ID NO:06 are provided. In another embodiment, CAR T cells comprising a polypeptide of SEQ ID NO:02, SEQ ID NO:05, or SEQ ID NO:07 are provided. In another illustrative aspect, a nucleic acid (e.g., an isolated nucleic acid) comprising SEQ ID NO:01, SEQ ID NO:03, SEQ ID NO:04, or SEQ ID NO:06 and encoding a chimeric antigen receptor is provided. In yet another embodiment, a chimeric antigen receptor polypeptide comprising SEQ ID NO:02, SEQ ID NO:05, or SEQ ID NO:07 is provided. In another embodiment, a vector is provided comprising SEQ ID NO:01, SEQ ID NO:03, SEQ ID NO:04 or SEQ ID NO:06. In another aspect, a lentiviral vector is provided comprising SEQ ID NO:01, SEQ ID NO:03, SEQ ID NO:04, or SEQ ID NO:06. In yet another embodiment, SEQ ID NO:02, SEQ ID NO:05, or SEQ ID NO:07 can comprise or consist of humanized, or human amino acid sequences.

In each of these embodiments, variant nucleic acid sequences or amino acid sequences having at least about 80%, at least about 90°%, at least about 95%, at least about 97%, at least about 98%, at least about 99%, or at least about 99.5% sequence identity to SEQ ID NOS:01 to 07 are contemplated. In another embodiment, the nucleic acid sequence can be a variant nucleic acid sequence having at least about 80%, at least about 90%, at least about 95%, at least about 97%, at least about 98%, at least about 99%, or at least about 99.5% sequence identity to SEQ ID NO:01, 03, 04, or 06 as long as the variant sequence encodes a polypeptide of SEQ ID NO:02 (for SEQ ID NO:1 and SEQ ID NO:03), SEQ ID NO:05 (for SEQ ID NO:04), or SEQ ID NO:07 (for SEQ ID NO:06). In another embodiment, the nucleic acid sequence or the amino acid sequence can be a variant nucleic acid or amino acid sequence having at least about 80%, at least about 90%, at least about 95%, at least about 97%, at least about 98%, at least about 99%, or at least about 99.5% sequence identity to SEQ ID NO:01, SEQ ID NO:03, SEQ ID NO:04, or SEQ ID NO:06 along a stretch of 200 nucleic acids or, for SEQ ID NO:02, SEQ ID NO:05, or SEQ ID NO:07 along a stretch of 200 amino acids. In one embodiment, determination of percent identity or similarity between sequences can be done, for example, by using the GAP program (Genetics Computer Group, software; now available via Accelrys), and alignments can be done using, for example, the ClustalW algorithm (VNTI software, InforMax Inc.). A sequence database can be searched using the nucleic acid or amino acid sequence of interest. Algorithms for database searching are typically based on the BLAST software (Altschul et al., 1990). In some embodiments, the percent identity can be determined along the full-length of the nucleic acid or amino acid sequence.

Also within the scope of the invention are nucleic acids complementary to the nucleic acids represented by SEQ ID NO:01, SEQ ID NO:03, SEQ ID NO:04, or SEQ ID NO:06 and those that hybridize to the nucleic acids represented by SEQ ID NO:01, SEQ ID NO:03, SEQ ID NO:04, or SEQ ID NO:06 or those that hybridize to their complements under highly stringent conditions. In accordance with the invention “highly stringent conditions” means hybridization at 65° C. in 5×SSPE and 50% formamide, and washing at 65° C. in 0.5×SSPE. Conditions for high stringency, low stringency and moderately stringent hybridization are described in Sambrook et al., “Molecular Cloning: A Laboratory Manual”, 3rd Edition, Cold Spring Harbor Laboratory Press, (2001), incorporated herein by reference, and Green and Sambrook, “Molecular Cloning: A Laboratory Manual”, 4th Edition. Cold Spring Harbor Laboratory Press, (2012), incorporated herein by reference. In some illustrative aspects, hybridization occurs along the full-length of the nucleic acid.

In one embodiment, the T lymphocytes (e.g., cytotoxic T lymphocytes used to prepare CAR T cells), used in the methods described herein, can be autologous cells, although heterologous cells can also be used, such as when the patient being treated has received high-dose chemotherapy or radiation treatment to destroy the patient's immune system. In one embodiment, allogenic cells can be used.

In one aspect, the T lymphocytes can be obtained from a patient by methods well-known in the art. For example, T cells (e.g., cytotoxic T cells) can be obtained by collecting peripheral blood from the patient, subjecting the blood to Ficoll density gradient centrifugation, and then using a negative T cell isolation kit (such as EasySep™ T Cell Isolation Kit) to isolate a population of T cells from the peripheral blood. In one illustrative embodiment, the population of T lymphocytes (e.g., cytotoxic T cells) need not be pure and may contain other cells such as other types of T cells (in the case of cytotoxic T cells, for example), monocytes, macrophages, natural killer cells, and B cells. In one aspect, the population being collected can comprise at least about 90% of the selected cell type, at least about 91%, 92%, 93%, 94%, 95%, 96%, 97%, 98%, 99%, or 100% of the selected cell type.

In one embodiment, after the T lymphocytes (e.g., cytotoxic T cells used to prepare CAR T cells) are obtained, the cells are cultured under conditions that promote the activation of the cells. In this embodiment, the culture conditions may be such that the cells can be administered to a patient without concern for reactivity against components of the culture medium. For example, the culture conditions may not include bovine serum products, such as bovine serum albumin. In one illustrative aspect, the activation can be achieved by introducing known activators into the culture medium, such as anti-CD3 antibodies in the case of cytotoxic T cells. Other suitable activators include anti-CD28 antibodies. In one aspect, the population of lymphocytes can be cultured under conditions promoting activation for about 1 to about 4 days. In one embodiment, the appropriate level of activation can be determined by cell size, proliferation rate, or activation markers determined by flow cytometry.

In one illustrative embodiment, after the population of T lymphocytes (e.g., cytotoxic T lymphocytes used to prepare CAR T cells) has been cultured under conditions promoting activation, the cells can be transfected with an expression vector encoding a CAR. Suitable vectors and transfection methods for use in various embodiments are described above. In one aspect, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. In one aspect, suitable culture conditions can be similar to the conditions under which the cells were cultured for activation either with or without the agent that was used to promote activation.

Thus, as described above, in one illustrative aspect, the methods of treatment described herein can further comprise 1) obtaining a population of autologous or heterologous T lymphocytes (e.g., cytotoxic T lymphocytes used to prepare CAR T cells), 2) culturing the T lymphocytes under conditions that promote the activation of the cells, and 3) transfecting the lymphocytes with an expression vector encoding a CAR to form CAR T cells.

In one embodiment, culture media that lacks any animal products, such as bovine serum, can be used to culture the CAR T cells. In another embodiment, tissue culture conditions typically used by the skilled artisan to avoid contamination with bacteria, fungi and mycoplasma can be used. In an exemplary embodiment, prior to being administered to a patient, the cells (e.g., CAR T cells) are pelleted, washed, and are resuspended in a pharmaceutically acceptable carrier or diluent. Exemplary compositions comprising CAR-expressing T lymphocytes (e.g., cytotoxic T lymphocytes) include compositions comprising the cells in sterile 290 mOsm saline, in infusible cryomedia (containing Plasma-Lyte A, dextrose, sodium chloride injection, human serum albumin and DMSO), in 0.9% NaCl with 2% human serum albumin, or in any other sterile 290 mOsm infusible materials. Alternatively, in another embodiment, depending on the identity of the culture medium, the CAR T cells can be administered in the culture media as the composition, or concentrated and resuspended in the culture medium before administration. In various embodiments, the CAR T cell composition can be administered to the patient via any suitable means, such as parenteral administration, e.g., intradermally, subcutaneously, intramuscularly, intraperitoneally, intravenously, or intrathecally.

In one aspect, the total number of CAR T cells and the concentration of the cells in the composition administered to the patient will vary depending on a number of factors including the type of T lymphocytes (e.g., cytotoxic T lymphocytes) being used, the binding specificity of the CAR, the identity of the targeting moiety and the small molecule ligand, the identity of the cancer, the location of the cancer in the patient, the means used to administer the compositions to the patient, and the health, age and weight of the patient being treated. In various embodiments, suitable compositions comprising transduced CAR T cells include those having a volume of about 0.1 ml to about 200 ml and about 0.1 ml to about 125 ml.

In various embodiments, the transduced CAR T cells administered to the patient can comprise from about 1×10⁵ to about 1×10¹⁵ or 1×10⁶ to about 1×10⁵ transduced CAR T cells. In various embodiments about 1×10⁵ to about 1×10¹⁰, about 1×10⁶ to about 1×10¹⁰, about 1×10⁶ to about 1×10⁹, about 1×10⁶ to about 1×10⁸, about 1×10⁶ to about 2×10⁷, about 1×10⁶ to about 3×10⁷, about 1×10⁶ to about 1.5×10⁷, about 1×10⁶ to about 1×10⁷, about 1×10 to about 9×10⁶, about 1×10⁶ to about 8×10⁶, about 1×10⁶ to about 7×10⁶, about 1×10⁶ to about 6×10⁶, about 1×10⁶ to about 5×10⁶, about 1×10⁶ to about 4×10⁶, about 1×10⁶ to about 3×10⁶, about 1×10⁶ to about 2×10⁶, about 2×10⁶ to about 6×10⁶, about 2×10⁶ to about 5×10⁶, about 3×10⁶ to about 6×10⁶, about 4×10⁶ to about 6×10⁶, about 4×10⁶ to about 1×10⁷, about 1×10⁶ to about 1×10⁷, about 1×10⁶ to about 1.5×10⁷, about 1×10⁶ to about 2×10⁷, about 0.2×10⁶ to about 1×10⁷, about 0.2×10⁶ to about 1.5×10⁷, about 0.2×10⁶ to about 2×10%, about 0.2×10⁶ to about 3×10⁷, about 0.2×10⁶ to about 4×10⁷, about 0.2×10⁶ to about 5×10⁷, about 0.2×10⁵ to about 1.5×10⁶, about 0.5×10⁵ to about 1.5×10⁶, about 0.2×10⁵ to about 1.4×10⁶, about 0.2×10⁵ to about 1.3×10⁶, about 0.5×10⁵ to about 1.3×10⁶, about 0.8×10⁶ to about 2×10⁷, about 0.8×10⁶ to about 1.5×10⁷, about 0.9×10⁶ to about 1.2×10⁷, or about 0.5×10⁶, 1×10⁶, 5×10⁶, 6×10⁶, 7×10⁶, 8×10⁶, 9×10⁶, 1×10⁷, 1.5×10⁷, 2×10⁷, 3×10⁷, 4×10⁷ or 5×10⁷ CAR T cells can be administered to the patient. These amounts can be per kg of patient body weight.

In other embodiments, the dose of the CAR T cells administered to the patient in the CAR T cell composition is selected from the group consisting of about 1 million, about 2 million, about 3 million, about 4 million, about 5 million, about 6 million, about 7 million, about 8 million, about 9 million, about 10 million, about 11 million, about 12 million, about 12.5 million, about 13 million, about 14 million, and about 15 million of the CAR T cells.

In any of the embodiments described in this paragraph, the CAR T cell dose can be in numbers of CAR T cells per kg of patient body weight. In one aspect, in any embodiment described herein, a single dose or multiple doses of the CAR T cells can be administered to the patient. In one illustrative embodiment, a first dose of the CAR T cells, and a second dose of the CAR T cells can be administered to the patient. In one aspect, the first dose of the CAR T cells can be a test dose to monitor the patient for tolerability to the CAR T cells, and the second dose of the CAR T cells can comprise a higher dose of the CAR T cells than the first dose of the CAR T cells. In one embodiment, the first dose of the CAR T cells can comprise about 0.5×10⁵ of the CAR T cells to about 1.5×10⁶ of the CAR T cells. In one embodiment, the first dose of the CAR T cells can comprise about or comprise 1×10⁶ of the CAR T cells. In another embodiment, the second dose of the CAR T cells can comprise about 0.8×10⁶ of the CAR T cells to about 2×10⁷ of the CAR T cells. In these embodiments involving a first and second dose of CAR T cells, any dose of CAR T cells described herein can be administered.

In any embodiment described herein, the CAR T cells can be administered before or after the compound, or the pharmaceutically acceptable salt thereof. As would be understood, the designations i), ii), and iii), etc. for steps of any method described herein do not indicate an order unless otherwise stated.

The compound, or pharmaceutically acceptable salt thereof, or CAR T cell composition described herein can be administered to the patient using any suitable method known in the art. As described herein, the term “administering” or “administered” includes all means of introducing the compound, or pharmaceutically acceptable salt thereof, or CAR T cell composition to the patient, including, but not limited to, oral, intravenous, intramuscular, subcutaneous, transdermal, and the like. In one aspect, the compound, or pharmaceutically acceptable salt thereof, described herein may be administered in a unit dosage form and/or formulations containing conventional nontoxic pharmaceutically acceptable carriers, adjuvants, and vehicles.

In one aspect, the compound, or pharmaceutically acceptable salt thereof, or CAR T cell composition as described herein may be administered directly into the blood stream, into muscle, or into an internal organ. In various embodiments, suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but they may be more suitably formulated as a sterile non-aqueous solution or as a dried form to be used in conjunction with a suitable vehicle such as sterile, pyrogen-free water or sterile saline. In other embodiments, any of the liquid formulations described herein may be adapted for parenteral administration as described herein. The preparation under sterile conditions, by lyophilization to produce a sterile lyophilized powder for a parenteral formulation, may readily be accomplished using standard pharmaceutical techniques well-known to those skilled in the art. In one embodiment, the solubility of the compound, or pharmaceutically acceptable salt thereof, used in the preparation of a parenteral formulation may be increased by the use of appropriate formulation techniques, such as the incorporation of solubility-enhancing agents.

In one embodiment, the amount of the compound, or pharmaceutically acceptable salt thereof, to be administered to the patient can vary significantly depending on the cancer being treated, the route of administration of the compound, or pharmaceutically acceptable salt thereof, and the tissue distribution. In one aspect, the amount to be administered to a patient can be based on body surface area, mass, and physician assessment.

In various embodiments, the compound, or the pharmaceutically acceptable salt thereof, can be administered in 1) at least a first dose escalation sequence, 2) at least a first dose escalation sequence and a second dose escalation sequence, 3) at least a first dose escalation sequence, a second dose escalation sequence, and a third dose escalation sequence, 4) at least a first dose escalation sequence, a second dose escalation sequence, a third dose escalation sequence, and a fourth dose escalation sequence, 5) at least a first dose escalation sequence, a second dose escalation sequence, a third dose escalation sequence, a fourth dose escalation sequence, and a fifth dose escalation sequence, 6) at least a first dose escalation sequence, a second dose escalation sequence, a third dose escalation sequence, a fourth dose escalation sequence, a fifth dose escalation sequence, and a sixth dose escalation sequence, or 7) one or more additional dose escalation sequences can be included.

In various embodiments, for the first, second, third, fourth, fifth, or sixth, etc. dose escalation sequence, the amount of the compound, or the pharmaceutically acceptable salt thereof, to be administered to the patient can be or be about 1 ug/kg to 50 ug/kg, or about 31 ug/kg or 31 ug/kg. In various embodiments, for the first, second, third, fourth, fifth, or sixth, etc. dose escalation sequence, the amount of the compound, or the pharmaceutically acceptable salt thereof, to be administered to the patient can be about 0.1 μg/kg to about 2000 μg/kg, 0.1 μg/kg to about 1500 μg/kg, 0.1 μg/kg to about 1000 μg/kg, 0.1 μg/kg to about 500 μg/kg, about 0.1 μg/kg to about 100 μg/kg, about 0.1 μg/kg to about 80 p/kg, about 0.1 μg/kg to about 70 μg/kg, about 0.1 μg/kg to about 50 μg/kg, about 0.1 μg/kg to about 40 μg/kg, about 0.1 μg/kg to about 30 μg/kg, about 0.3 μg/kg to about 500 μg/kg, about 0.3 μg/kg to about 400 μg/kg, about 0.3 μg/kg to about 300 μg/kg, about 0.3 μg/kg to about 200 μg/kg, about 0.3 μg/kg to about 100 μg/kg, about 0.3 μg/kg to about 90 μg/kg, about 0.1 μg/kg to about 400 μg/kg, about 0.1 μg/kg to about 350 μg/kg, about 0.1 μg/kg to about 300 μg/kg, about 0.1 μg/kg to about 250 μg/kg, about 0.1 μg/kg to about 200 μg/kg, about 0.1 μg/kg to about 150 μg/kg, or about 0.3 μg/kg to about 150 μg/kg, or 5 μg/kg to about 2000 μg/kg, 5 μg/kg to about 1500 μg/kg, 5 μg/kg to about 1000 μg/kg, 5 μg/kg to about 500 μg/kg, about 5 μg/kg to about 100 μg/kg, about 5 μg/kg to about 80 p/kg, about 5 μg/kg to about 70 μg/kg, about 5 μg/kg to about 50 μg/kg, about 5 μg/kg to about 40 μg/kg, or about 5 μg/kg to about 30 μg/kg. In another embodiment, for the first and fourth dose escalation sequences, the amount of the compound, or the pharmaceutically acceptable salt thereof, to be administered to the patient can be about 0.1 μg/kg to about 100 μg/kg, about 0.1 μg/kg to about 80 μg/kg, about 0.1 μg/kg to about 70 μg/kg, about 0.1 μg/kg to about 50 μg/kg, about 0.1 μg/kg to about 40 μg/kg, about 0.1 μg/kg to about 30 μg/kg, or about 0.3 μg/kg to about 30 μg/kg, about 5 μg/kg to about 100 μg/kg, about 5 μg/kg to about 80 μg/kg, about 5 μg/kg to about 70 μg/kg, about 5 μg/kg to about 50 μg/kg, about 5 μg/kg to about 40 μg/kg, about 5 μg/kg to about 30 μg/kg, or about 5 μg/kg to about 2000 μg/kg, or about 5 μg/kg to about 1500 μg/kg, or about 5 μg/kg to about 1000 μg/kg, or about 5 μg/kg to about 500 μg/kg. In another embodiment, for the second and fifth dose escalation sequences, the amount of the compound, or the pharmaceutically acceptable salt thereof, to be administered to the patient can be about 0.3 μg/kg to about 500 μg/kg, about 0.3 μg/kg to about 400 μg/kg, about 0.3 μg/kg to about 300 μg/kg, about 0.3 μg/kg to about 200 μg/kg, about 0.3 μg/kg to about 100 μg/kg, or about 0.3 μg/kg to about 90 μg/kg, or about 5 μg/kg to about 100 μg/kg, about 5 μg/kg to about 80 jg/kg, about 5 μg/kg to about 70 μg/kg, about 5 μg/kg to about 50 μg/kg, about 5 μg/kg to about 40 μg/kg, about 5 μg/kg to about 30 μg/kg, or about 5 μg/kg to about 2000 μg/kg, or about 5 μg/kg to about 1500 μg/kg, or about 5 μg/kg to about 1000 μg/kg, or about 5 μg/kg to about 500 μg/kg. In yet another embodiment, for the third and sixth dose escalation sequences, the amount of the compound, or the pharmaceutically acceptable salt thereof, to be administered to the patient can be 0.1 μg/kg to about 400 μg/kg, about 0.1 μg/kg to about 350 μg/kg, about 0.1 μg/kg to about 300 μg/kg, about 0.1 μg/kg to about 250 μg/kg, about 0.1 μg/kg to about 200 μg/kg, about 0.1 μg/kg to about 150 μg/kg, or about 0.3 μg/kg to about 150 μg/kg, or about 5 μg/kg to about 100 μg/kg, about 5 μg/kg to about 80 μg/kg, about 5 μg/kg to about 70 μg/kg, about 5 μg/kg to about 50 μg/kg, about 5 μg/kg to about 40 μg/kg, about 5 μg/kg to about 30 μg/kg, or about 5 μg/kg to about 2000 μg/kg, or about 5 μg/kg to about 1500 μg/kg, or about 5 μg/kg to about 1000 μg/kg, or about 5 μg/kg to about 500 μg/kg. In these embodiments, “kg” is kilograms of body weight of the patient.

In any of these embodiments, the range of amounts of the compound, or the pharmaceutically acceptable salt thereof, can be a range based on calculating percentages of a “full dose” of the compound, or the pharmaceutically acceptable salt thereof, wherein a “full dose” of the compound, or the pharmaceutically acceptable salt thereof, can be about 30 μg/kg, and wherein the percentages are about 1 percent to about 100 percent (see Sequence 1 in FIG. 1), about 1 percent to about 300 percent (see Sequence 2 in FIG. 1), and about 1 percent to about 500 percent (see Sequence 3 in FIG. 1) of the “full dose” of the compound, or the pharmaceutically acceptable salt thereof. In other embodiments, the percentages of the “full dose” of the compound, or the pharmaceutically acceptable salt thereof, administered in a dose escalation sequence can be about 1 percent, about 2 percent, about 3 percent, about 4 percent, about 5 percent, about 6 percent, about 7 percent, about 8 percent, about 9 percent, about 10 percent, about 20 percent, about 30 percent, about 40 percent, about 50 percent, about 60 percent, about 70 percent, about 80 percent, about 90 percent, about 100 percent, about 200 percent, about 300 percent, about 400 percent, or about 500 percent, and the amounts administered can be about 0.3 μg/kg, about 3 μg/kg, about 9 μg/kg, about 15 μg/kg, about 30 μg/kg, about 90 μg/kg, or about 150 μg/kg, respectively or about 17 percent, about 333 percent, about 1666 percent, or about 3333 percent, and the amounts administered can be about 5 μg/kg, about 100 μg/kg, about 500 μg/kg, or about 1000 μg/kg, respectively. In these embodiments, “kg” is kilograms of body weight of the patient.

In another embodiment, the percentages of the “full dose” of the compound, or the pharmaceutically acceptable salt thereof, administered in a dose escalation sequence can be about 1 percent, about 2 percent, and about 20 percent for the first dose escalation sequence, about 1 percent, about 6 percent, and about 60 percent for the second dose escalation sequence, and about 1 percent, about 10 percent, and about 100 percent for the third dose escalation sequence, and the “full dose” of the compound, or the pharmaceutically acceptable salt thereof, can be 500 nmoles/kg. In this embodiment, “kg” is kilograms of body weight of the patient. In this embodiment, the compound, or the pharmaceutically acceptable salt thereof, can be administered on varying days with about can be repeated one, two, three, four, five, six, seven, eight, nine, or ten times or any appropriate number of days between each dose escalation cycle. In this embodiment, the compound, or the pharmaceutically acceptable salt thereof, can be administered on varying days with about 6 days between each dose escalation cycle. In this embodiment, the compound, or the pharmaceutically acceptable salt therefor, can be administered on day 4, day 7, day 11, day 18, and day 25 following the first dose. In this embodiment, the compound, or the pharmaceutically acceptable salt thereof, can be administered on any one of days 26-32 following the first dose.

In another embodiment, the percentages of the “full dose” of the compound, or the pharmaceutically acceptable salt thereof, administered in a dose escalation sequence can be about 1 percent, about 5 percent, about 10 percent, about 30 percent, about 50 percent, about 100 percent, about 200 percent, about 300 percent, about 400 percent, about 500 percent, about 600 percent, or any integer that is between 1 and 600 percent, for the first dose escalation sequence. In this embodiment, the percentages of the “full dose” of the compound, or the pharmaceutically acceptable salt thereof, administered in a dose escalation sequence can be about 1 percent, about 5 percent, about 10 percent, about 20 percent, about 30 percent, about 50 percent, about 100 percent, about 200 percent, about 300 percent, about 400 percent, about 500 percent, about 600 percent, or any integer that is between 1 and 600 percent, for the second dose escalation sequence. In this embodiment, the “full dose” of the compound, or the pharmaceutically acceptable salt thereof, can be 500 nmoles/kg. In this embodiment, “kg” is kilograms of body weight of the patient. In this embodiment, the compound, or the pharmaceutically acceptable salt thereof, can be administered on varying days with about can be repeated one, two, three, four, five, six, seven, eight, nine, or ten times or any appropriate number of days between each dose escalation cycle. In this embodiment, the compound, or the pharmaceutically acceptable salt thereof, can be administered on varying days with about 6 days between each dose escalation cycle. In this embodiment, the compound, or the pharmaceutically acceptable salt therefor, can be administered on day 4, day 7, day 11, day 18, and day 25 following the first dose. In this embodiment, the compound, or the pharmaceutically acceptable salt thereof, can be administered on any one of days 26-32 following the first dose. In other embodiments, the dose escalations can be repeated one, two, three, four, five, six, seven, eight, nine, or ten times or any appropriate number of times.

In another embodiment, the percentages of the “full dose” of the compound, or the pharmaceutically acceptable salt thereof, administered in the first or fourth dose escalation sequence can be about 1 percent, about 10 percent, and about 100 percent of the “full dose” of the compound, or the pharmaceutically acceptable salt thereof, (about 10 to about 50, about 20 to about 40, about 25 to about 35, or about 30 μg/kg) in escalating amounts, and the amounts of the compound, or the pharmaceutically acceptable salt thereof, administered can be about 0.3 μg/kg, about 3 μg/kg, and about 30 μg/kg, respectively. In yet another embodiment, the percentages of the “full dose” of the compound, or the pharmaceutically acceptable salt thereof, administered in the second or fifth dose escalation sequence can be about 1 percent, about 30 percent, and about 300 percent of the “full dose” of the compound, or the pharmaceutically acceptable salt thereof, (about 10 to about 50, about 20 to about 40, about 25 to about 35, or about 30 μg/kg) in escalating amounts, and the amounts of the compound, or the pharmaceutically acceptable salt thereof, administered can be about 0.3 μg/kg, about 9 μg/kg, and about 90 μg/kg, respectively. In still another embodiment, the percentages of the “full dose” of the compound, or the pharmaceutically acceptable salt thereof, administered in the third or sixth dose escalation sequence can be about 1 percent, about 50 percent, and about 500 percent of the “full dose” of the compound, or the pharmaceutically acceptable salt thereof, (about 10 to about 50, about 20 to about 40, about 25 to about 35, or about 30 μg/kg) in escalating amounts, and the amounts of the compound, or the pharmaceutically acceptable salt thereof, administered can be about 0.3 μg/kg, about 15 μg/kg, and about 150 μg/kg, respectively. In these embodiments, “kg” is kilograms of body weight of the patient.

In various other embodiments, amounts to be administered to the patient can range, for example, from about 0.05 mg to about 30 mg, 0.05 mg to about 25.0 mg, about 0.05 mg to about 20.0 mg, about 0.05 mg to about 15.0 mg, about 0.05 mg to about 10.0 mg, about 0.05 mg to about 9.0 mg, about 0.05 mg to about 8.0 mg, about 0.05 mg to about 7.0 mg, about 0.05 mg to about 6.0 mg, about 0.05 mg to about 5.0 mg, about 0.05 mg to about 4.0 mg, about 0.05 mg to about 3.0 mg, about 0.05 mg to about 2.0 mg, about 0.05 mg to about 1.0 mg, about 0.05 mg to about 0.5 mg, about 0.05 mg to about 0.4 mg, about 0.05 mg to about 0.3 mg, about 0.05 mg to about 0.2 mg, about 0.05 mg to about 0.1 mg, about 0.01 mg to about 2 mg, about 0.3 mg to about 10 mg, about 0.1 mg to about 20 mg, or about 0.8 to about 3 mg.

In other embodiments, the dose of the compound, or pharmaceutically acceptable salt thereof, can range, for example, from about 50 nmoles/kg to about 3000 nmoles/kg of patient body weight, about 50 nmoles/kg to about 2000 nmoles/kg, about 50 nmoles/kg to about 1000 nmoles/kg, about 50 nmoles/kg to about 900 nmoles/kg, about 50 nmoles/kg to about 800 nmoles/kg, about 50 nmoles/kg to about 700 nmoles/kg, about 50 nmoles/kg to about 600 nmoles/kg, about 50 nmoles/kg to about 500 nmoles/kg, about 50 nmoles/kg to about 400 nmoles/kg, about 50 nmoles/kg to about 300 nmoles/kg, about 50 nmoles/kg to about 200 nmoles/kg, about 50 nmoles/kg to about 100 nmoles/kg, about 100 nmoles/kg to about 300 nmoles/kg, about 100 nmoles/kg to about 500 nmoles/kg, about 100 nmoles/kg to about 1000 nmoles/kg, about 100 nmoles/kg to about 2000 nmoles/kg of patient body weight. In other embodiments, the dose may be about 1 nmoles/kg, about 5 nmoles/kg, about 10 nmoles/kg, about 20 nmoles kg, about 25 nmoles/kg, about 30 nmoles/kg, about 40 nmoles/kg, about 50 nmoles/kg, about 60 nmoles/kg, about 70 nmoles/kg, about 80 nmoles/kg, about 90 nmoles/kg, about 100 nmoles/kg, about 150 nmoles/kg, about 200 nmoles/kg, about 250 nmoles/kg, about 300 nmoles/kg, about 350 nmoles/kg, about 400 nmoles/kg, about 450 nmoles/kg, about 500 nmoles/kg, about 600 nmoles/kg, about 700 nmoles/kg, about 800 nmoles/kg, about 900 nmoles/kg, about 1000 nmoles/kg, about 2000 nmoles/kg, about 2500 nmoles/kg or about 3000 nmoles/kg of body weight of the patient. In yet other embodiments, the dose may be about 0.1 nmoles/kg, about 0.2 nmoles/kg, about 0.3 nmoles/kg, about 0.4 nmoles kg, or about 0.5 nmoles/kg, about 0.1 nmoles/kg to about 1000 nmoles/kg, about 0.1 nmoles/kg to about 900 nmoles/kg, about 0.1 nmoles/kg to about 850 nmoles/kg, about 0.1 nmoles/kg to about 800 nmoles/kg, about 0.1 nmoles/kg to about 700 nmoles/kg, about 0.1 nmoles/kg to about 600 nmoles/kg, about 0.1 nmoles/kg to about 500 nmoles/kg, about 0.1 nmoles/kg to about 400 nmoles/kg, about 0.1 nmoles/kg to about 300 nmoles/kg, about 0.1 nmoles/kg to about 200 nmoles/kg, about 0.1 nmoles/kg to about 100 nmoles/kg, about 0.1 nmoles/kg to about 50 nmoles/kg, about 0.1 nmoles/kg to about 10 nmoles/kg, or about 0.1 nmoles/kg to about 1 nmoles/kg of body weight of the patient. In other embodiments, the dose may be about 0.3 nmoles/kg to about 1000 nmoles/kg, about 0.3 nmoles/kg to about 900 nmoles/kg, about 0.3 nmoles/kg to about 850 nmoles/kg, about 0.3 nmoles/kg to about 800 nmoles/kg, about 0.3 nmoles/kg to about 700 nmoles/kg, about 0.3 nmoles/kg to about 600 nmoles/kg, about 0.3 nmoles/kg to about 500 nmoles/kg, about 0.3 nmoles/kg to about 400 nmoles/kg, about 0.3 nmoles/kg to about 300 nmoles/kg, about 0.3 nmoles/kg to about 200 nmoles/kg, about 0.3 nmoles/kg to about 100 nmoles/kg, about 0.3 nmoles/kg to about 50 nmoles/kg, about 0.3 nmoles/kg to about 10 nmoles/kg, or about 0.3 nmoles/kg to about 1 nmoles/kg of body weight of the patient. In these embodiments, “kg” is kilograms of body weight of the patient.

In another embodiment, a first dose escalation step and a second dose escalation step with the compound (e.g., where each dose escalation step is 50 nmol/kg and then 500 nmol/kg), or a pharmaceutically acceptable salt thereof, are performed after administration of CAR-T cells (e.g., in any of the amounts described herein). In this embodiment, after the first and second dose escalation steps in, for example, weeks one and two, the level of the compound, or a pharmaceutically acceptable salt thereof, can be kept constant in week three relative to the last dose administered in week two (e.g., kept constant at 500 nmol/kg). In any embodiment described herein, the level of the compound, or a pharmaceutically acceptable salt thereof, can be kept constant in the succeeding week relative to the last dose administered in the prior week.

In various other embodiments, the dose of the compound, or the pharmaceutically acceptable salt thereof, may range from, for example, about 10 nmoles/kg to about 10000 nmoles/kg, from about 10 nmoles/kg to about 5000 nmoles/kg, from about 10 nmoles/kg to about 3000 nmoles/kg, about 10 nmoles/kg to about 2500 nmoles/kg, about 10 nmoles/kg to about 2000 nmoles/kg, about 10 nmoles/kg to about 1000 nmoles/kg, about 10 nmoles/kg to about 900 nmoles/kg, about 10 nmoles/kg to about 800 nmoles/kg, about 10 nmoles/kg to about 700 nmoles/kg, about 10 nmoles/kg to about 600 nmoles/kg, about 10 nmoles/kg to about 500 nmoles/kg, about 10 nmoles/kg to about 400 nmoles/kg, about 10 nmoles/kg to about 300 nmoles/kg, about 10 nmoles/kg to about 200 nmoles/kg, about 10 nmoles/kg to about 150 nmoles/kg, about 10 nmoles/kg to about 100 nmoles/kg, about 10 nmoles/kg to about 90 nmoles/kg, about 10 nmoles/kg to about 80 nmoles/kg, about 10 nmoles/kg to about 70 nmoles/kg, about 10 nmoles/kg to about 60 nmoles/kg, about 10 nmoles/kg to about 50 nmoles/kg, about 10 nmoles/kg to about 40 nmoles/kg, about 10 nmoles/kg to about 30 nmoles/kg, about 10 nmoles/kg to about 20 nmoles/kg, about 200 nmoles/kg to about 900 nmoles/kg, about 200 nmoles/kg to about 800 nmoles/kg, about 200 nmoles/kg to about 700 nmoles/kg, about 200 nmoles/kg to about 600 nmoles/kg, about 200 nmoles/kg to about 500 nmoles/kg, about 250 nmoles/kg to about 600 nmoles/kg, about 300 nmoles/kg to about 600 nmoles/kg, about 300 nmoles/kg to about 500 nmoles/kg, or about 400 nmoles/kg to about 600 nmoles/kg, of body weight of the patient. In these embodiments, “kg” is kilograms of body weight of the patient.

In all of the dose embodiments described above, the percentages of the “full dose” of the compound, or the pharmaceutically acceptable salt thereof, administered at any step in a dose escalation sequence can be about 1 percent, about 2 percent, about 3 percent, about 4 percent, about 5 percent, about 6 percent, about 7 percent, about 8 percent, about 9 percent, about 10 percent, about 20 percent, about 30 percent, about 40 percent, about 50 percent, about 60 percent, about 70 percent, about 80 percent, about 90 percent, about 100 percent, about 200 percent, about 300 percent, about 400 percent, or about 500 percent of the “full dose” of the compound, or the pharmaceutically acceptable salt thereof. The “full dose” of the compound, or the pharmaceutically acceptable salt thereof, can be any of the doses of the compound, or the pharmaceutically acceptable salt thereof, described in the preceding paragraphs as being doses administered in a dose escalation sequence.

In various other embodiments, the dose of the compound, or the pharmaceutically acceptable salt thereof, may range from, for example, about 1 nmoles/kg to about 10000 nmoles/kg, from about 1 nmoles/kg to about 5000 nmoles/kg, from about 1 nmoles/kg to about 3000 nmoles/kg, about 1 nmoles/kg to about 2500 nmoles/kg, about 1 nmoles/kg to about 2000 nmoles/kg, about 1 nmoles/kg to about 1000 nmoles/kg, about 1 nmoles/kg to about 900 nmoles/kg, about 1 nmoles/kg to about 800 nmoles/kg, about 1 nmoles/kg to about 700 nmoles/kg, about 1 nmoles/kg to about 600 nmoles/kg, about 1 nmoles/kg to about 500 nmoles/kg, about 1 nmoles/kg to about 400 nmoles/kg, about 1 nmoles/kg to about 300 nmoles/kg, about 1 nmoles/kg to about 200 nmoles/kg, about 1 nmoles/kg to about 150 nmoles/kg, about 1 nmoles/kg to about 100 nmoles/kg, about 1 nmoles/kg to about 90 nmoles/kg, about 1 nmoles/kg to about 80 nmoles/kg, about 1 nmoles/kg to about 70 nmoles/kg, about 1 nmoles/kg to about 60 nmoles/kg, about 1 nmoles/kg to about 50 nmoles/kg, about 1 nmoles/kg to about 40 nmoles/kg, about 1 nmoles/kg to about 30 nmoles/kg, or about 1 nmoles/kg to about 20 nmoles/kg, In these embodiments, “kg” is kilograms of body weight of the patient.

In another embodiment, from about 20 ug/kg of body weight of the patient to about 3 mg/kg of body weight of the patient of the compound, or the pharmaceutically acceptable salt thereof, can be administered to the patient. In another aspect, amounts can be from about 0.2 mg/kg of body weight of the patient to about 0.4 mg/kg of body weight of the patient.

In any of the above-described dose embodiments, a single dose or multiple doses of the compound, or pharmaceutically acceptable salt thereof, may be administered to the patient.

In one embodiment, the small molecule ligand linked to the targeting moiety can be administered to the patient before the CAR T cell composition. In another embodiment, the small molecule ligand linked to the targeting moiety can be administered to the patient at the same time as the CAR T cell composition, but in different formulations, or in the same formulation. In yet another embodiment, the small molecule ligand linked to the targeting moiety can be administered to the patient after the CAR T cell composition.

In one illustrative aspect, the timing between the administration of CAR T cells and the small molecule linked to the targeting moiety may vary widely depending on factors that include the type of CAR T cells being used, the binding specificity of the CAR, the identity of the targeting moiety and the small molecule ligand, the identity of the cancer, the location in the patient of the cancer, the method used to administer to the patient the CAR T cells and the small molecule ligand linked to the targeting moiety, and the health, age, and weight of the patient. In one aspect, the small molecule ligand linked to the targeting moiety can be administered before or after the CAR T cells, such as within about 3, 6, 9, 12, 15, 18, 21, 24, 27, 30, 33, 36, 39, 42, 45, 48, or 51 hours, or within about 0.5, 1, 1.5, 2, 2.5, 3, 45, 6, 7, 8, 9, 10 or more days.

In one embodiment, any applicable dosing schedule known in the art can be used for administration of the compound, or the pharmaceutically acceptable salt thereof, or for the CAR T cell composition. In one aspect, the dosing schedule selected for the compound, or the pharmaceutically acceptable salt thereof, and the CAR T cell composition can take into consideration the concentration of the compound, or the pharmaceutically acceptable salt thereof, and the number of CAR T cells administered, to regulate the cytotoxicity of the CAR T cell composition and to control CRS.

In one exemplary embodiment, the first dose escalation sequence, the second dose escalation sequence, the third dose escalation sequence, the fourth dose escalation sequence, the fifth dose escalation sequence, the sixth dose escalation sequence, or any additional dose escalation sequence can be followed by a period of time during which the compound, or the pharmaceutically acceptable salt thereof, is not administered. In various illustrative embodiments, the period of time that the compound, or the pharmaceutically acceptable salt thereof, is not administered can be 1, 2, 3, 4, 5, 6, 7, 8, 9, or 10 days. In another embodiment, the period of time that the compound, or the pharmaceutically acceptable salt thereof, is not administered can be 6, 7, or 8 days. In yet another embodiment, the period of time that the compound, or the pharmaceutically acceptable salt thereof, is not administered can be 7 days.

In one aspect, a first dose of the CAR T cells and a second dose of the CAR T cells are administered to the patient during week 1, for example, on Monday and Thursday. In this embodiment, the first dose escalation sequence for the compound, or the pharmaceutically acceptable salt thereof, can then occur during weeks 2 and 3. For example, the compound, or the pharmaceutically acceptable salt thereof, can be administered on three separate days and the three separate days can be Monday and Thursday of week 2 and Monday of week 3 (see Sequence 1 in FIG. 1). In this embodiment, the second dose escalation sequence for the compound, or the pharmaceutically acceptable salt thereof, can then occur during weeks 4 and 5 (see Sequence 2 in FIG. 1). For example, the compound, or the pharmaceutically acceptable salt thereof, can be administered on three separate days and the three separate days can be Monday and Thursday of week 4 and Monday of week 5. In this embodiment, the third dose escalation sequence for the compound, or the pharmaceutically acceptable salt thereof, can then occur during weeks 6 and 7 (see Sequence 3 in FIG. 1). For example, the compound, or the pharmaceutically acceptable salt thereof, can be administered on three separate days and the three separate days can be Monday and Thursday of week 6 and Monday of week 7. In other embodiments, subsequent dose escalation sequences can follow a similar sequence, or Sequence 3 can be repeated about 7 days after Sequence 3 ends as “Course 2” and no additional treatments with the compound, or the pharmaceutically acceptable salt thereof, occur until the beginning of “Course 3” (the length of “Course 2” being based on the length of time shown in FIG. 1 for “Course” 1). In one embodiment, the patient can receive one, two, three, or four courses of therapy.

In another illustrative embodiment, a method of treatment or inhibition of a cancer is provided. The method comprises i) administering to a patient at least one dose of a CAR T cell composition comprising CAR T cells wherein the CAR T cells comprise a CAR directed to a targeting moiety, ii) administering to the patient a compound, or a pharmaceutically acceptable salt thereof, wherein the compound comprises a small molecule ligand linked to a targeting moiety by a linker and wherein the compound, or the pharmaceutically acceptable salt thereof, is administered in a first dose escalation sequence wherein, if serious CRS occurs in the first dose escalation sequence, the compound, or the pharmaceutically acceptable salt thereof, is administered using a lower dose escalation sequence wherein the first dose of the compound, or the pharmaceutically acceptable salt thereof, in the lower dose escalation sequence is lower than the first dose of the compound, or the pharmaceutically acceptable salt thereof, administered in the first dose escalation sequence. In this embodiment, in the lower dose escalation sequence, the compound, or the pharmaceutically acceptable salt thereof, is administered at about 0.5 percent, about 5 percent, and about 50 percent of a full dose of the compound, or the pharmaceutically acceptable salt thereof, on three separate days. In this embodiment, the full dose of the compound, or the pharmaceutically acceptable salt thereof, can be about 10 μg/kg to about 50 μg/kg, about 20 μg/kg to about 40 μg/kg, about 25 μg/kg to about 35 μg/kg, or about 30 μg/kg. In these embodiments, “kg” is kilograms of body weight of the patient.

In various related embodiments, lymphocytes can be depleted in the patient before administration of the CAR T cell composition to the patient using, for example, cytoxan, fludarabine, and/or etopside, and the method can further comprise additional steps including, but not limited to, administering platelets to the patient, administering packed red blood cells to the patient, administering cryoprecipitate to the patient, administering intravenous immunoglobulin to the patient, treating with calcium supplements, acid-citrate-dextrose, and/or heparin, and/or providing antimicrobial therapy to the patient. In one embodiment, lymphodepletion occurs at least about 24 hours prior to CAR T cell administration.

In another aspect, the method can comprise CRS monitoring steps. In one illustrative aspect, if no CRS or neurotoxicity is observed in the patient during the first dose escalation sequence, the method can be advanced to the second dose escalation sequence. If no CRS or neurotoxicity is observed in the patient during the second dose escalation sequence, the method can be advanced to the third dose escalation sequence. If no CRS or neurotoxicity is observed in the patient during the third dose escalation sequence, the method can be advanced to the fourth dose escalation sequence. If no CRS or neurotoxicity is observed in the patient during the fourth dose escalation sequence, the method can be advanced to the fifth dose escalation sequence. If no CRS or neurotoxicity is observed in the patient during the fifth dose escalation sequence, the method can be advanced to the sixth dose escalation sequence, and so on.

In another illustrative embodiment, if fever without hypotension (i.e., non-serious CRS) is observed in the patient and no neurotoxicity is observed in the patient during any one of the dose escalation sequences, all subsequent doses of the compound, or the pharmaceutically acceptable salt thereof, can be administered to the patient at the dose escalation sequence level that caused the fever without hypotension. In another aspect, if CRS (i.e., serious CRS) or neurotoxicity occurs in the patient in any dose escalation sequence, all subsequent doses of the compound, or the pharmaceutically acceptable salt thereof, can be administered to the patient at the dose escalation sequence level below the dose escalation sequence level that caused the serious CRS or neurotoxicity in the patient.

In one aspect, serious CRS may include any toxicity requiring the use of cetuximab, any ≥grade 3 autoimmune toxicity, any ≥grade 3 toxicity that may be attributed to CAR T cell administration or administration of the compound, or the pharmaceutically acceptable salt thereof, and that occurs within 28 days following initiation of treatment except ≤grade 4 fever lasting for less than 48 hours after initiation of treatment, ≤grade 3 chills lasting for less than 24 hours after initiation of treatment, 5 grade 3 cough lasting for less than 24 hours after initiation of treatment, ≤grade 3 transaminases lasting for less than 7 days after initiation of treatment, ≤grade 3 hypotension lasting for less than 48 hours after initiation of treatment, ≤grade 3 CRS lasting for less than 48 hours after initiation of treatment, 5 grade 3 anaphylaxis related to DMSO resolvable with Benadryl and/or epinephrine, or ≤grade 3 pain controlled with oral or IV narcotic therapy, or, in addition, except for toxicities occurring about 3 weeks after the CAR T cell infusion including 5 grade 3 chills lasting up to 5 days, ≤grade 3 transaminases lasting up to 2 weeks, ≤grade 3 CRS lasting up to 2 weeks, ≤grade 4 lymphopenia, ≤grade 4 leukopenia, or 5 grade 3 pain lasting for up to 2 weeks controlled with oral or IV narcotic therapy.

In one embodiment, to prevent or inhibit CRS in the patient, the method can further comprise the step of administering to the patient a folate, a conjugate comprising a folate wherein the conjugate comprising a folate does not comprise a targeting moiety, or a drug that inhibits activation of the CAR T cells. In this embodiment, any of a folate, a conjugate comprising a folate wherein the conjugate comprising a folate does not comprise a targeting moiety, or a drug that inhibits activation of the CAR T cells can be referred to herein as “a rescue agent”. In one embodiment, a folate, such as folic acid, can be administered to prevent or inhibit CRS in the patient. In this embodiment, the folate inhibits interaction of the bridge (i.e., the small molecule ligand linked to the targeting moiety by a linker) with the receptors for the bridge on the tumor inhibiting tumor lysis and preventing or inhibiting CRS in the patient.

In one embodiment, the folate administered as an inhibitor of binding of the bridge to the tumor can be, for example, folic acid, a folic acid analog, or another folate receptor-binding molecule. In various embodiments, analogs of folate that can be used include folinic acid, pteropolyglutamic acid, and folate receptor-binding pteridines such as tetrahydropterins, dihydrofolates, tetrahydrofolates, or their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, or 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, or 3′,5′-dichloro-4-amino-4-deoxy-NlO-methylpteroylglutamic acid (dichloromethotrexate).

In another embodiment, the folate administered as an inhibitor of binding of the bridge to the tumor has the formula:

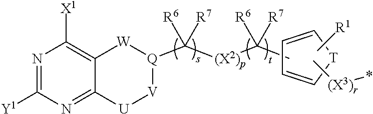

wherein X¹ and Y¹ are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{4b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents an optional covalent bond to the rest of the conjugate, if any additional chemical moieties are part of the folate.

In yet another embodiment, a conjugate comprising a folate can be administered to prevent or inhibit cytokine release syndrome (CRS) in the patient. CRS can cause detrimental effects to the patient, including, but not limited to weight loss, high fever, pulmonary edema, and a dangerous drop in blood pressure.

In this embodiment, the conjugate comprising a folate does not comprise a targeting moiety, and, thus, the conjugate inhibits interaction of the bridge with the tumor to prevent tumor lysis and reduce CRS in the patient. In this embodiment, the folate moiety in the conjugate comprising a folate can comprise any of the folates described in the preceding paragraphs linked to a chemical moiety that does not comprise a targeting moiety. In one aspect, the conjugate comprising a folate can comprise a folate linked to one or more amino acids that do not comprise a targeting moiety. Illustratively, the conjugate comprising a folate can have the formula:

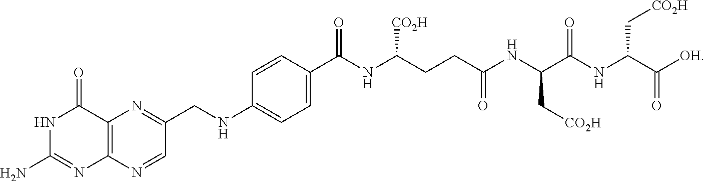 [SMILES: Nc1nc2ncc(CNc3ccc(C(=O)N[C@@H](CCC(=O)N[C@H](CC(=O)O)C(=O)N[C@H](CC(=O)O)C(=O)O)C(=O)O)cc3)nc2c(=O)[nH]1]

This compound can also be referred to as “EC923”. In these embodiments, the folate or the conjugate comprising a folate can be administered to the patient in molar excess relative to the bridge (i.e., the small molecule ligand linked to a targeting moiety by a linker), such as a 10-fold excess, a 100-fold excess, a 200-fold excess a 300-fold excess a 400-fold excess a 500-fold excess a 600-fold excess a 700-fold excess a 800-fold excess a 900-fold excess, a 1000-fold excess, or a 10,000-fold excess of the folate or the conjugate comprising a folate relative to the small molecule ligand linked to a targeting moiety by a linker. The amount of the folate or the conjugate comprising a folate relative to the amount of the small molecule ligand linked to a targeting moiety by a linker needed to inhibit interaction of the bridge with the tumor can be determined by the skilled artisan.

In another embodiment, an agent that inhibits activation of the CAR T cells can be administered to the patient to inhibit CAR T cell activation and to inhibit or prevent CRS in the patient. In one aspect the agent can be selected from the group consisting of a lymphocyte-specific protein tyrosine kinase inhibitor (e.g., Dasatinib), a PI3 kinase inhibitor (e.g., GDC0980), Tociluzumab, an inhibitor of an IL-2 inducible T cell kinase (e.g., BMS-509744), JAK inhibitors, BTK inhibitors, SIP agonists (e.g. Siponimod and Ozanimod), and an agent that blocks CAR T cell binding to the bridge, but does not bind to the cancer (e.g., fluoresceinamine, FITC, or sodium fluorescein). It is understood by the skilled artisan that FITC (i.e., fluorescein) can be in the form of a salt (e.g., sodium fluorescein), or in its unsalted form, under physiological conditions or, for example, in a buffer at physiological pH. Accordingly, in one embodiment, when fluorescein is administered to a patient it may be in equilibrium between its salted form (e.g., sodium fluorescein) and its unsalted form. In another embodiment, a rescue agent that inhibits activation of CAR T cells can be a compound of the formula:

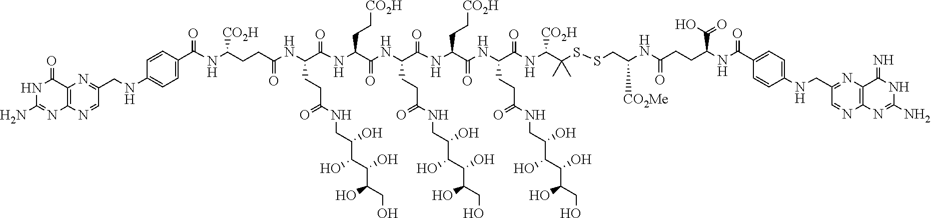 [SMILES: COC(=O)[C@H](CSSC(C)(C)[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CC[C@H](NC(=O)c1ccc(NCc2cnc3nc(N)[nH]c(=O)c3n2)cc1)C(=O)O)C(=O)O)NC(=O)CC[C@H](NC(=O)c1ccc(NCc2cnc3nc(N)[nH]c(=N)c3n2)cc1)C(=O)O]

This compound can also be referred to as “EC2319”.

In various embodiments, the rescue agent can be administered at a concentration of from about 0.001 nM to about 100 mM, about 0.01 nM to about 100 mM, about 1 nM to about 100 mM, about 10 nM to about 100 mM, about 50 nM to about 100 mM, or from about 100 nM to about 100 mM in any appropriate volume, including, for example, 0.1 ml, 0.2 ml, 0.3 ml, 0.4 ml, 0.5 ml, 0.6 ml, 0.7 ml, 0.8 ml, 0.9 ml, 1 ml, 2 ml, 3 ml, 4 ml, 5 ml, 10 ml, 100 ml, or 1000 ml. In other embodiments, the rescue agent can be administered at a dose of about 0.01 to about 300 umoles/kg of body weight of the patient, about 0.06 to about 100 umoles/kg of body weight of the patient, about 0.06 to about 90 umoles/kg of body weight of the patient, about 0.06 to about 80 umoles/kg of body weight of the patient, about 0.06 to about 70 umoles/kg of body weight of the patient, about 0.06 to about 60 umoles/kg of body weight of the patient, about 0.06 to about 50 umoles/kg of body weight of the patient, about 0.06 to about 40 umoles/kg of body weight of the patient, about 0.06 to about 30 umoles/kg of body weight of the patient, about 0.06 to about 20 umoles/kg of body weight of the patient, about 0.06 to about 10 umoles/kg of body weight of the patient, about 0.06 to about 8 umoles/kg of body weight of the patient, or about 0.06 to about 6 umoles/kg of body weight of the patient.

In these embodiments, the rescue agent can be administered to the patient in molar excess relative to the compound, or its pharmaceutically acceptable salt (i.e., the small molecule ligand linked to a targeting moiety by a linker), such as about a 10-fold excess, about a 20-fold excess, about a 30-fold excess, about a 40-fold excess, about a 50-fold excess, about a 60-fold excess, about a 70-fold excess, about a 80-fold excess, about a 90-fold excess, about a 100-fold excess, about a 200-fold excess, about a 300-fold excess, about a 400-fold excess, about a 500-fold excess, about a 600-fold excess, about a 700-fold excess, about a 800-fold excess, about a 900-fold excess, about a 1000-fold excess, or about a 10,000-fold excess of the rescue agent relative to the small molecule ligand linked to a targeting moiety by a linker. The amount of the rescue agent relative to the amount of the small molecule ligand linked to a targeting moiety by a linker needed to inhibit interaction of the compound, or its pharmaceutically acceptable salt, with the tumor and/or the CAR T cells can be determined by the skilled artisan.

In another embodiment, more than one dose can be administered to the patient of the folate, the conjugate comprising a folate wherein the conjugate comprising a folate does not comprise a targeting moiety, or the agent that inhibits activation of the CAR T cells.

In the ‘rescue agent’ embodiments described herein, the folate, the conjugate comprising a folate wherein the conjugate comprising a folate does not comprise a targeting moiety, or the agent that inhibits activation of the CAR T cells can be administered to the patient before and/or after the compound, or the pharmaceutically acceptable salt thereof. In another aspect, the compound, or the pharmaceutically acceptable salt thereof, can be administered before and subsequent to administration of the folate, the conjugate comprising a folate wherein the conjugate comprising a folate does not comprise a targeting moiety, or the agent that inhibits activation of the CAR T cells. In this embodiment, the subsequent administration of the compound, or the pharmaceutically acceptable salt thereof, can cause CAR T cell activation and an increase in cytokine levels in the patient.

In another embodiment, administration of the folate, the conjugate comprising a folate wherein the conjugate comprising a folate does not comprise a targeting moiety, or the agent that inhibits activation of the CAR T cells can cause reduction in cytokine levels in the patient. In another embodiment, the reduction in cytokine levels is a reduction to about the cytokine levels in an untreated patient. In other embodiments, the agent that inhibits activation of the CAR T cells is administered to the patient when the CRS grade reaches 1, 2, 3, or 4 or when the CRS grade reaches 3 or 4.

In any of the embodiments described herein where a folate is the ligand linked to the targeting moiety by a linker, the patient can be put on a folate deficient diet prior to treatment with the methods described herein, or the patient can be administered a folate in the diet. In the embodiment where the patient is administered folate, the dose can range, for example, from about 50 nmol/kg to about 3000 nmol/kg of patient body weight, about 50 nmol/kg to about 2000 nmol/kg, about 50 nmol/kg to about 1000 nmol/kg, about 50 nmol/kg to about 900 nmol/kg, about 50 nmol/kg to about 800 nmol/kg, about 50 nmol/kg to about 700 nmol/kg, about 50 nmol/kg to about 600 nmol/kg, about 50 nmol/kg to about 500 nmol/kg, about 50 nmol/kg to about 400 nmol/kg, about 50 nmol/kg to about 300 nmol/kg, about 50 nmol/kg to about 200 nmol/kg, about 50 nmol/kg to about 100 nmol/kg, about 100 nmol/kg to about 300 nmol/kg, about 100 nmol/kg to about 500 nmol/kg, about 100 nmol/kg to about 1000 nmol/kg, about 100 nmol/kg to about 2000 nmol/kg of patient body weight. In other embodiments, the dose may be about 100 nmol/kg, about 150 nmol/kg, about 200 nmol/kg, about 250 nmol/kg, about 300 nmol/kg, about 350 nmol/kg, about 400 nmol/kg, about 450 nmol/kg, about 500 nmol/kg, about 600 nmol/kg, about 700 nmol/kg, about 800 nmol/kg, about 900 nmol/kg, about 1000 nmol/kg, about 2000 nmol/kg, or about 3000 nmol/kg of patient body weight. In these embodiments, “kg” is kilograms of patient body weight. In one aspect, the folate can be administered, for example, daily, weekly, biweekly, three times a week, or using any suitable regimen for administration of the folate.

In various embodiments described herein, the CAR T cells can persist in elevated numbers of circulating CAR T cells for as long as about 10 days, as long as about 15 days, as long as about 20 days, as long as about 25 days, as long as about 30 days, as long as about 35 days, as long as about 40 days, as long as about 45 days, as long as about 50 days, as long as about 55 days, as long as about 60 days, as long as about 65 days, as long as about 70 days, as long as about 75 days, or as long as about 80 days post CAR T cell administration.

In various embodiments described herein, half-maximal effective concentrations (EC50) for the compound, or the pharmaceutically acceptable salt thereof, can be about 1 pM to about 2 nM, about 1 pM to about 5 nM, about 1 pM to about 10 nM, about 1 pM to about 20 nM, about 1 pM to about 30 nM, about 1 pM to about 40 nM, about 1 pM to about 50 nM, about 1 pM to about 60 nM, about 1 pM to about 70 nM, about 1 pM to about 80 nM, about 1 pM to about 90 nM, about 1 pM to about 100 nM, about 1 pM to about 200 nM, about 1 pM to about 300 nM, about 1 pM to about 400 nM, about 1 pM to about 500 nM, about 1 pM to about 600 nM, about 1 pM to about 700 nM, about 1 pM to about 800 nM, about 1 pM to about 900 nM, about 1 pM to about 1 nM, about 1 pM to about 900 pM, about 1 pM to about 800 pM, about 1 pM to about 700 pM, about 1 pM to about 600 pM, about 1 pM to about 500 pM, about 1 pM to about 400 pM, about 1 pM to about 300 pM, about 1 pM to about 200 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, or about 1 pM to about 5 pM.

In various embodiments described herein, the Kd for binding of the compound, or the pharmaceutically acceptable salt thereof, to the CAR T cells can be about 1 nM to about 100 nM, about 1 nM to about 200 nM, about 1 nM to about 300 nM, about 1 nM to about 400 nM, about 1 nM to about 500 nM, about 1 nM to about 600 nM, about 1 nM to about 700 nM, about 1 nM to about 800 nM, about 1 nM to about 900 nM, about 100 nM to about 500 nM, about 100 nM to about 400 nM, about 100 nM to about 300 nM, about 100 nM to about 200 nM, about 100 nM to about 150 nM, or about 130 nM.

In the various embodiments described herein, EGFRt-sorted or unsorted CAR T cells can be used. In another embodiment, a “clinical facsimile” batch of CAR T cells can be used with a low differentiation profile. In another embodiment, a “research batch” of CAR T cells can be used. The “clinical facsimile” batch (~39% EGFRt+) can comprise CD4+ subsets at about 66% TSCM and about 32% TCM and CD8 subsets at about 95% TSCM and about 3% TCM. The research batch (~23% EGFRt+) can comprise CD4 subsets at about 32% TSCM, about 53% TCM, about 11% TEM and about 3.7% TEFF and CD8 subsets at about 44% TSCM, about 0.28% TCM, about 3.4% TEM or about 52% TEFF.

In various illustrative embodiments described herein, the compound, or the pharmaceutically acceptable salt thereof, can be first administered to the patient about 1, about 2, about 3, about 4, about 5, about 6, about 7, about 8, about 9, or about 10 days before or after the CAR T cells, or on any appropriate day before or after the CAR T cells.

In one embodiment of the methods described herein, the cancer is imaged prior to administration to the patient of the compound, or the pharmaceutically acceptable salt thereof, or prior to administration of the CAR T cell composition to the patient. In one illustrative embodiment, imaging occurs by PET imaging. In other illustrative embodiments imaging occurs by MRI imaging or SPECT/CT imaging. The imaging method can be any suitable imaging method known in the art. In one embodiment, the imaging method can involve the use of the small molecule ligand described herein but linked to an imaging agent suitable for the types of imaging described herein to determine if the patient is positive for folate receptor expression. In another embodiment, immunohistochemical analysis can be used for this purpose.

In any of the embodiments described herein, cytokine release resulting in off-target toxicity in the patient may not occur even though CAR T cell toxicity to the cancer occurs. In any embodiment described herein, off-target tissue toxicity may not occur in the patient even though CAR T cell toxicity to the cancer occurs. In any embodiment described herein, the cancer may comprise a tumor, and tumor size may be reduced in the patient, even though off-target toxicity does not occur. In any of the embodiments described herein. CRS can be reduced or prevented, and the method can result in a decrease in tumor volume in the patient. In any embodiment described herein, body weight loss due to CRS, and CAR T cell exhaustion can be reduced or prevented. In any embodiment described herein, the cancer can comprise a tumor and a complete response for the tumor may be obtained.

### Certain Therapeutic Methods and Compositions

Some embodiments of the methods and compositions provided herein include methods of treating, ameliorating or inhibiting an osteosarcoma in a human subject. Some such methods include administering to the subject a fluorescein isothiocyanate (FITC)-folate conjugate, or a pharmaceutically acceptable salt thereof, in a dosing regimen comprising: a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, (c) a third dose of about 50% to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg; wherein the human subject has received or is receiving a chimeric antigen receptor (CAR) T cell composition comprising a population of T cells expressing an anti-fluorescein CAR, thereby treating the osteosarcoma in the human subject.

Some embodiments of the methods and compositions provided herein include methods of treating, ameliorating or inhibiting an osteosarcoma in a human subject. Some such methods include administering to the subject a fluorescein isothiocyanate (FITC)-folate conjugate, or a pharmaceutically acceptable salt thereof, in a dosing regimen comprising: (i) a dosing escalation cycle to identify a maximum tolerated dose (MTD) of the FITC-folate conjugate, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, and (c) a third dose of about 50% to about 500% of the full dose of the FITC-folate conjugate, or a pharmaceutically acceptable salt thereof, wherein the full dose of the FITC-folate conjugate or a pharmaceutically acceptable salt thereof, is about 1×10⁻² mg/kg to about 5×10⁻² mg/kg; and (ii) a stable dosing cycle comprising administering the MTD of the FITC-folate conjugate, or a pharmaceutically acceptable salt thereof, in a dosing interval of once every about 5-10 days for a duration of time; wherein the human subject has received or is receiving a CAR T cell composition comprising a population of T cells expressing an anti-fluorescein CAR, thereby treating, ameliorating or inhibiting the osteosarcoma in the human subject.

In some embodiments, the osteosarcoma is recurring. In some embodiments, the osteosarcoma is refractory.

In some embodiments, the period of time between administering the first dose and administering the second dose is between about 1 to about 7 days. In some embodiments, the period of time between administering the first dose and administering the second dose is about 2 days.

In some embodiments, the period of time between administering the second dose and administering the third dose is between about 1 to about 7 days. In some embodiments, the period of time between administering the second dose and administering the third dose is about 3 days.

In some embodiments, the full dose of the FITC-folate conjugate, or the pharmaceutically acceptable salt thereof, is about 3.1×10⁻² mg/kg.

Some embodiments also include administering a fifth dose of the MTD of the FITC-folate conjugate, or a pharmaceutically acceptable salt thereof. Some embodiments also include a restaging period after the end of the fifth dose, wherein the restaging period comprises: assessing the subject to determine if one or more criteria for receiving at least one subsequent dosing cycle of the FITC-folate conjugate, or the pharmaceutically acceptable salt thereof, is met, provided the subject is not administered the FITC-folate conjugate during the restaging period.

In some embodiments, the subject meets one or more criteria, and wherein the subject is administered at least one subsequent dosing cycle comprising administering the MTD of the FITC-folate conjugate, or the pharmaceutically acceptable salt thereof, in a dosing interval of once every about 5-10 days for a duration of time.

In some embodiments, the anti-fluorescein CAR comprises a fully humanized anti-fluorescein antibody or antigen binding fragment thereof. In some embodiments, the anti-fluorescein CAR comprises a fully humanized anti-fluorescein scFv. In some embodiments, the anti-fluorescein CAR comprises a fully humanized E2 anti-fluorescein scFv. In some embodiments, the CAR T cells express a cell surface selectable marker comprising a truncated EGFR (EGFRt) polypeptide.

In some embodiments, the dose of the CAR T cell composition is between about 1×10⁵ cells/kg to about 1×10⁷ cells/kg.

In some embodiments, the FITC-folate conjugate, or the pharmaceutically acceptable salt thereof, is administered intravenously.

Some embodiments of the methods and compositions provided herein include methods of treating, ameliorating or inhibiting a cancer in a human subject comprising: (i) administering to the subject a first dose of a fluorescein isothiocyanate (FITC)-folate conjugate, or a pharmaceutically acceptable salt thereof; (ii) administering to the subject a second dose of the FITC-folate conjugate, or the pharmaceutically acceptable salt thereof, wherein the second dose is higher than the first dose; (iii) administering to the subject a third dose of the FITC-folate conjugate, or the pharmaceutically acceptable salt thereof, wherein the third dose is higher than the second dose, wherein a maximum tolerated dose (MTD) of the FITC-folate conjugate, or a pharmaceutically acceptable salt thereof, is identified after the third dose; and (iv) administering to the subject a fourth dose of the MTD of the FITC-folate conjugate, or a pharmaceutically acceptable salt thereof; wherein the human subject has received or is receiving a CAR T cell composition comprising a population of T cells expressing an anti-fluorescein CAR, thereby treating, ameliorating or inhibiting the cancer in the human subject.

Some embodiments of the methods and compositions provided herein include methods of treating, ameliorating or inhibiting an osteosarcoma in a human subject, comprising administering to the subject a fluorescein isothiocyanate (FITC)-folate conjugate, or a pharmaceutically acceptable salt thereof in combination with a CAR T cell therapy, wherein the FITC-folate conjugate is administered to the subject in an escalating dosing regimen subsequent to or concurrently with administration of the CAR T cell therapy to the subject, thereby treating the osteosarcoma in the human subject.

In some embodiments, the escalating dosing regimen comprises: a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 500% of the full dose of the FITC-folate conjugate, or a pharmaceutically acceptable salt thereof, (c) a third dose of about 50%/6 to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10-2 mg/kg to about 5×10-2 mg/kg;

In some embodiments, the escalating dosing regimen comprises: (i) a dosing escalation cycle to identify a maximum tolerated dose (MTD) of the FITC-folate conjugate, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, and (c) a third dose of about 50% to about 500% of the full dose of the FITC-folate conjugate, or a pharmaceutically acceptable salt thereof, wherein the full dose of the FITC-folate conjugate or a pharmaceutically acceptable salt thereof, is about 1×10-2 mg/kg to about 5×10-2 mg/kg; and (ii) a stable dosing cycle comprising administering the MTD of the FITC-folate conjugate, or a pharmaceutically acceptable salt thereof, in a dosing interval of once every about 5-10 days for a duration of time.

In some embodiments, the CAR T cell therapy comprises administering to the subject a population of CAR T cells expressing an anti-fluorescein CAR

Some embodiments of the methods and compositions provided herein include use of a fluorescein isothiocyanate (FITC)-folate conjugate, or a pharmaceutically acceptable salt thereof for treating, ameliorating or inhibiting an osteosarcoma in a human subject in combination with a CAR T cell therapy, wherein the FITC-folate conjugate is administered to the subject in an escalating dosing regimen subsequent to or concurrently with administration of the CAR T cell therapy to the subject.

In some embodiments, the escalating dosing regimen comprises a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 500% of the full dose of the FITC-folate conjugate, or a pharmaceutically acceptable salt thereof, (c) a third dose of about 50% to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg.

In some embodiments, the escalating dosing regimen comprises: (i) a dosing escalation cycle to identify a maximum tolerated dose (MTD) of the FITC-folate conjugate, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, and (c) a third dose of about 50% to about 500% of the full dose of the FITC-folate conjugate, or a pharmaceutically acceptable salt thereof, wherein the full dose of the FITC-folate conjugate or a pharmaceutically acceptable salt thereof, is about 1×10⁻² mg/kg to about 5×10⁻² mg/kg; and (ii) a stable dosing cycle comprising administering the MTD of the FITC-folate conjugate, or a pharmaceutically acceptable salt thereof, in a dosing interval of once every about 5-10 days for a duration of time.

In some embodiments, the escalating dosing regimen comprises: (i) a dosing escalation cycle to identify a maximum tolerated dose (MTD) of the FITC-folate conjugate, wherein the dosing escalation cycle comprises administering: (a) a first dose of about 0.5% or about 1% of a full dose of the FITC-folate conjugate, or a pharmaceutically acceptable salt thereof, (b) a second dose of about 5%, about 100, about 30%, or about 50% of the full dose of the FITC-folate conjugate, or a pharmaceutically acceptable salt thereof, and (c) a third dose of about 50%, about 100%, about 300%, or about 500% of the full dose of the FITC-folate conjugate, or a pharmaceutically acceptable salt thereof, wherein the full dose of the FITC-folate conjugate or a pharmaceutically acceptable salt thereof, is about 1×10² mg/kg to about 5×10² mg/kg; and (ii) a stable dosing cycle comprising administering the MTD of the FITC-folate conjugate, or a pharmaceutically acceptable salt thereof, in a dosing interval of once every about 5-10 days for a duration of time. In these embodiments, the dosing regiment may be (1) a first dose of 0.5% of a full dose of the FITC-folate conjugate, or a pharmaceutically acceptable salt thereof, a second dose of 5% of a full dose of the FITC-folate conjugate, or a pharmaceutically acceptable salt thereof, and a third dose of 50% of a full dose of the FITC-folate conjugate, or a pharmaceutically acceptable salt thereof; (2) a first dose of 1% of a full dose of the FITC-folate conjugate, or a pharmaceutically acceptable salt thereof, a second dose of 10% of a full dose of the FITC-folate conjugate, or a pharmaceutically acceptable salt thereof, and a third dose of 100% of a full dose of the FITC-folate conjugate, or a pharmaceutically acceptable salt thereof; (3) a first dose of 1% of a full dose of the FITC-folate conjugate, or a pharmaceutically acceptable salt thereof, a second dose of 30% of a full dose of the FITC-folate conjugate, or a pharmaceutically acceptable salt thereof, and a third dose of 300% of a full dose of the FITC-folate conjugate, or a pharmaceutically acceptable salt thereof; or (4) a first dose of 1% of a full dose of the FITC-folate conjugate, or a pharmaceutically acceptable salt thereof, a second dose of 50% of a full dose of the FITC-folate conjugate, or a pharmaceutically acceptable salt thereof, and a third dose of 500% of a full dose of the FITC-folate conjugate, or a pharmaceutically acceptable salt thereof.

Some embodiments of the methods and compositions provided herein include use of a fluorescein isothiocyanate (FITC)-folate conjugate, or a pharmaceutically acceptable salt thereof for treating, ameliorating or inhibiting a cancer in a human subject in combination with a CAR T cell therapy, wherein the FITC-folate conjugate, or a pharmaceutically acceptable salt thereof is administered in a method comprising: (i) administering to the subject a first dose of the FITC-folate conjugate, or a pharmaceutically acceptable salt thereof; (ii) administering to the subject a second dose of the FITC-folate conjugate, or the pharmaceutically acceptable salt thereof, wherein the second dose is higher than the first dose; (iii) administering to the subject a third dose of the FITC-folate conjugate, or the pharmaceutically acceptable salt thereof, wherein the third dose is higher than the second dose, wherein a maximum tolerated dose (MTD) of the FITC-folate conjugate, or a pharmaceutically acceptable salt thereof, is identified after the third dose; and (iv) administering to the subject a fourth dose of the MTD of the FITC-folate conjugate, or a pharmaceutically acceptable salt thereof. In some embodiments, the cancer comprises an osteosarcoma. In some embodiments, the FITC-folate conjugate is administered to the subject in an escalating dosing regimen subsequent to or concurrently with administration of the CAR T cell therapy to the subject. In some embodiments, the CAR T cell therapy comprises administration to the subject a population of CAR T cells expressing an anti-fluorescein CAR.

### Certain Kits and Systems

Some embodiments of the methods and compositions provided herein include systems and kits comprising a container comprising a fluorescein isothiocyanate (FITC)-folate conjugate, or pharmaceutically acceptable salt thereof, and optionally a pharmaceutically carrier, wherein the kit comprises instructions for treating, ameliorating or inhibiting osteosarcoma in a subject that has received or is receiving a CAR T cell composition comprising CAR T cells, wherein treating, ameliorating or inhibiting comprises administering the FITC-folate conjugate in a dosing regimen comprising: a dosing escalation cycle to identify a maximum tolerated dose (MTD) of the FITC-folate conjugate, or a pharmaceutically acceptable salt thereof, wherein the dosing escalation cycle comprises administering: (a) a first dose of about 0.5% to about 5% of a full dose of the FITC-folate conjugate, or a pharmaceutically acceptable salt thereof, (b) a second dose of about 5% to about 50% of the full dose of the FITC-folate conjugate, or a pharmaceutically acceptable salt thereof, (c) a third dose of about 50% to about 500% of the full dose of the FITC-folate conjugate, or a pharmaceutically acceptable salt thereof, wherein the MTD is identified after the third dose, and (d) a fourth dose of the MTD of the FITC-folate conjugate, or a pharmaceutically acceptable salt thereof, wherein the full dose of the FITC-folate conjugate, or a pharmaceutically acceptable salt thereof, is about 1×10⁻² mg/kg to about 5×10⁻² mg/kg, and wherein the CAR comprises an anti-fluorescein antibody or antigen binding fragment thereof. In some embodiments, the container is a vial.

Some embodiments of the methods and compositions provided herein include systems for treating, ameliorating or inhibiting osteosarcoma in a subject with a fluorescein isothiocyanate (FITC)-folate conjugate, or pharmaceutically acceptable salt thereof in combination with a CAR T cell therapy, wherein the FITC-folate conjugate is administered to the subject in an escalating dosing regimen subsequent to or concurrently with administration of the CAR T cell therapy to the subject. In some embodiments, the system comprises: (a) a first dose of about 0.5% to about 5% of a full dose of the FITC-folate conjugate, or a pharmaceutically acceptable salt thereof: (b) a second dose of about 5% to about 50% of the full dose of the FITC-folate conjugate, or a pharmaceutically acceptable salt thereof; (c) a third dose of about 50% to about 500% of the full dose of the FITC-folate conjugate, or a pharmaceutically acceptable salt thereof, wherein a maximum tolerated dose (MTD) of the FITC-folate conjugate, or a pharmaceutically acceptable salt thereof is identified after the third dose; and (d) a fourth dose of the MTD of the FITC-folate conjugate, or a pharmaceutically acceptable salt thereof. In some embodiments, the full dose of the FITC-folate conjugate, or a pharmaceutically acceptable salt thereof, is about 1×10⁻² mg/kg to about 5×10⁻² mg/kg. Some embodiments also include a population of CAR T cells expressing an anti-fluorescein CAR.

Certain polynucleotide and polypeptide sequences useful with embodiments provided herein are listed in TABLE 1.

TABLE 1

| SEQ ID NO: | Sequence |
|---|---|
| SEQ ID | ATGGCCTTACCAGTGACCGCCTTGCTCCTGCCGCTGGCCTTGCT |
| NO: 01 | GCTCCACGCCGCCAGGCCGGATGTCGTGATGACCCAGACCCCCC |
| | TCAGCCTCCCAGTGTCCCTCGGTGACCAGGCTTCTATTAGTTGC |
| | AGATCCAGCCAGTCCCTCGTGCACTCTAACGGTAATACCTACCT |
| | GAGATGGTATCTCCAGAAGCCCGGACAGAGCCCTAAGGTGCTG |
| | ATCTACAAAGTCTCCAACCGGGTGTCTGGAGTCCCTGACCGCTT |
| | CTCAGGGAGCGGTTCCGGCACCGACTTCACCCTGAAGATCAACC |
| | GGGTGGAGGCCGAAGACCTCGGCGTCTATTTCTGCTCTCAGAGT |
| | ACACATGTGCCCTGGACCTTCGGCGGAGGGACCAAGCTGGAGA |
| | TCAAAAGCTCCGCAGACGATGCCAAGAAAGATGCCGCTAAGAA |
| | AGACGATGCTAAGAAAGACGATGCAAAGAAAGACGGTGGCGTG |
| | AAGCTGGATGAAACCGGAGGAGGTCTCGTCCAGCCAGGAGGAG |
| | CCATGAAGCTGAGTTGCGTGACCAGCGGATTCACCTTTGGGCAC |
| | TACTGGATGAACTGGGTGCGACAGTCCCCAGAGAAGGGGCTCG |
| | AATGGGTCGCTCAGTTCAGGAACAAACCCTACAATTATGAGAC |
| | ATACTATTCAGACAGCGTGAAGGGCAGGTTTACTATCAGTAGAG |
| | ACGATTCCAAATCTAGCGTGTACCTGCAGATGAACAATCTCAGG |
| | GTCGAAGATACAGGCATCTACTATTGCACAGGGGCATCCTATGG |
| | TATGGAGTATCTCGGTCAGGGGACAAGCGTCACAGTCAGTTTCG |
| | TGCCGGTCTTCCTGCCAGCGAAGCCCACCACGACGCCAGCGCCG |
| | CGACCACCAACACCGGCGCCCACCATCGCGTCGCAGCCCCTGTC |
| | CCTGCGCCCAGAGGCGTGCCGGCCAGCGGCGGGGGGCGCAGTG |
| | CACACGAGGGGGCTGGACTTCGCCTGTGATATCTACATCTGGGC |
| | GCCCTTGGCCGGGACTTGTGGGGTCCTTCTCCTGTCACTGGTTAT |
| | CACCCTTTACTGCAACCACAGGAACCGTTTCTCTGTTGTTAAAC |
| | GGGGCAGAAAGAAACTCCTGTATATATTCAAACAACCATTTATG |
| | AGACCAGTACAAACTACTCAAGAGGAAGATGGCTGTAGCTGCC |
| | GATTTCCAGAAGAAGAAGAAGGAGGATGTGAACTGAGAGTGAA |
| | GTTCAGCAGGAGCGCAGACGCCCCCGCGTACCAGCAGGGCCAG |
| | AACCAGCTCTATAACGAGCTCAATCTAGGACGAAGAGAGGAGT |
| | ACGATGTTTTGGACAAGAGACGTGGCCGGGACCCTGAGATGGG |
| | GGGAAAGCCGAGAAGGAAGAACCCTCAGGAAGGCCTGTACAAT |
| | GAACTGCAGAAAGATAAGATGGCGGAGGCCTACAGTGAGATTG |
| | GGATGAAAGGCGAGCGCCGGAGGGGCAAGGGGCACGATGGCC |
| | TTTACCAGGGTCTCAGTACAGCCACCAAGGACACCTACGACGCC |
| | CTTCACATGCAGGCCCTGCCCCCTCGCTAA |
| SEQ ID | MALPVTALLLPLALLLHAARPDVVMTQTPLSLPVSLGDQASISCRS |
| NO: 02 | SQSLVHSNGNTYLRWYLQKPGQSPKVLIYKVSNRVSGVPDRFSGS |
| | GSGTDFTLKINRVEAEDLGVYFCSQSTHVPWTFGGGTKLEIKSSAD |
| | DAKKDAAKKDDAKKDDAKKDGGVKLDETGGGLVQPGGAMKLS |
| | CVTSGFTFGHYWMNWVRQSPEKGLEWVAQFRNKPYNYETYYSDS |
| | VKGRFTISRDDSKSSVYLQMNNLRVEDTGIYYCTGASYGMEYLGQ |
| | GTSVTVSFVPVFLPAKPTTTPAPRPPTPAPTIASQPLSLRPEACRPAA |
| | GGAVHTRGLDFACDIYIWAPLAGTCGVLLLSLVITLYCNHRNRFSV |
| | VKRGRKKLLYIFKQPFMRPVQTTQEEDGCSCRFPEEEEGGCELRVK |
| | FSRSADAPAYQQGQNQLYNELNLGRREEYDVLDKRRGRDPEMGG |
| | KPRRKNPQEGLYNELQKDKMAEAYSEIGMKGERRRGKGHDGLYQ |
| | GLSTATKDTYDALHMQALPPR |
| SEQ ID | GCCACCATGGCCTTACCAGTGACCGCCTTGCTCCTGCCGCTGGC |
| NO: 03 | CTTGCTGCTCCACGCCGCCAGGCCGGATGTCGTGATGACCCAGA |
| | CCCCCCTCAGCCTCCCAGTGTCCCTCGGTGACCAGGCTTCTATTA |
| | GTTGCAGATCCAGCCAGTCCCTCGTGCACTCTAACGGTAATACC |
| | TACCTGAGATGGTATCTCCAGAAGCCCGGACAGAGCCCTAAGG |
| | TGCTGATCTACAAAGTCTCCAACCGGGTGTCTGGAGTCCCTGAC |
| | CGCTTCTCAGGGAGCGGTTCCGGCACCGACTTCACCCTGAAGAT |
| | CAACCGGGTGGAGGCCGAAGACCTCGGCGTCTATTTCTGCTCTC |
| | AGAGTACACATGTGCCCTGGACCTTCGGCGGAGGGACCAAGCT |
| | GGAGATCAAAAGCTCCGCAGACGATGCCAAGAAAGATGCCGCT |
| | AAGAAAGACGATGCTAAGAAAGACGATGCAAAGAAAGACGGT |
| | GGCGTGAAGCTGGATGAAACCGGAGGAGGTCTCGTCCAGCCAG |
| | GAGGAGCCATGAAGCTGAGTTGCGTGACCAGCGGATTCACCTTT |
| | GGGCACTACTGGATGAACTGGGTGCGACAGTCCCCAGAGAAGG |
| | GGCTCGAATGGGTCGCTCAGTTCAGGAACAAACCCTACAATTAT |
| | GAGACATACTATTCAGACAGCGTGAAGGGCAGGTTTACTATCA |
| | GTAGAGACGATTCCAAATCTAGCGTGTACCTGCAGATGAACAAT |
| | CTCAGGGTCGAAGATACAGGCATCTACTATTGCACAGGGGCATC |
| | CTATGGTATGGAGTATCTCGGTCAGGGGACAAGCGTCACAGTCA |
| | GTTTCGTGCCGGTCTTCCTGCCAGCGAAGCCCACCACGACGCCA |
| | GCGCCGCGACCACCAACACCGGCGCCCACCATCGCGTCGCAGC |
| | CCCTGTCCCTGCGCCCAGAGGCGTGCCGGCCAGCGGCGGGGGG |
| | CGCAGTGCACACGAGGGGGCTGGACTTCGCCTGTGATATCTACA |
| | TCTGGGCGCCCTTGGCCGGGACTTGTGGGGTCCTTCTCCTGTCA |
| | CTGGTTATCACCCTTTACTGCAACCACAGGAACCGTTTCTCTGTT |
| | GTTAAACGGGGCAGAAAGAAACTCCTGTATATATTCAAACAAC |
| | CATTTATGAGACCAGTACAAACTACTCAAGAGGAAGATGGCTG |
| | TAGCTGCCGATTTCCAGAAGAAGAAGAAGGAGGATGTGAACTG |
| | AGAGTGAAGTTCAGCAGGAGCGCAGACGCCCCCGCGTACCAGC |
| | AGGGCCAGAACCAGCTCTATAACGAGCTCAATCTAGGACGAAG |
| | AGAGGAGTACGATGTTTTGGACAAGAGACGTGGCCGGGACCCT |
| | GAGATGGGGGGAAAGCCGAGAAGGAAGAACCCTCAGGAAGGC |
| | CTGTACAATGAACTGCAGAAAGATAAGATGGCGGAGGCCTACA |
| | GTGAGATTGGGATGAAAGGCGAGCGCCGGAGGGGCAAGGGGC |
| | ACGATGGCCTTTACCAGGGTCTCAGTACAGCCACCAAGGACACC |
| | TACGACGCCCTTCACATGCAGGCCCTGCCCCCTCGCTAA |
| SEQ ID | ATGCTTCTCCTGGTGACAAGCCTTCTGCTCTGTGAGTTACCACAC |
| NO: 04 | CCAGCATTCCTCCTGATCCCAAGCGTGCTGACACAGCCTAGCTC |
| E2 anti- | CGTGTCTGCCGCCCCTGGCCAGAAAGTGACCATCAGCTGTAGCG |
| fluorescein | GCAGCACCAGCAACATCGGCAACAACTACGTGTCCTGGTATCA |
| CAR | GCAGCACCCCGGCAAGGCCCCCAAGCTGATGATCTACGACGTG |
| The initial | TCCAAGCGGCCCAGCGGCGTGCCCGATAGATTTTCCGGCAGCAA |
| start codon is | GAGCGGCAACAGCGCCAGCCTGGATATCAGCGGCCTGCAGTCT |
| underlined | GAGGACGAGGCCGACTACTATTGCGCCGCCTGGGACGATAGCC |
| and begins at | TGAGCGAGTTCCTGTTTGGCACCGGCACCAAGCTGACAGTGCTG |
| nucleotide 1. | GGCGGAGGCGGAGGATCTGGCGGCGGAGGAAGTGGCGGAGGG |
| The internal | GGATCTCAGGTGCAGCTGGTGGAAAGCGGCGGCAACCTGGTGC |
| sequence | AGCCTGGCGGATCTCTGAGACTGAGCTGTGCCGCCAGCGGCTTC |
| beginning at | ACCTTCGGCAGCTTCAGCATGAGCTGGGTGCGCCAGGCTCCTGG |
| ‘AGC’ to | GGGAGGACTGGAATGGGTGGCAGGACTGAGCGCCAGAAGCAGC |
| ‘GGC’ (both | CTGACCCACTACGCCGATAGCGTGAAGGGCCGGTTCACCATCAG |
| underlined | CCGGGACAACGCCAAGAACAGCGTGTACCTGCAGATGAACAGC |
| and bold) | CTGCGGGTGGAAGATACCGCCGTGTACTACTGCGCCAGACGGTC |
| encodes the | CTACGACAGCAGCGGCTACTGGGGCCACTTCTACAGCTACATGG |
| expressed | ACGTGTGGGGCCAGGGCACCCTCGTGACAGTGTCTGAGAGCAA |
| CAR. | GTACGGACCGCCCTGCCCCCCTTGCCCTGCCCCCGAGTTCGACG |
| | GCGGACCCAGCGTGTTCCTGTTCCCCCCCAAGCCCAAGGACACC |
| | CTGATGATCAGCCGGACCCCCGAGGTGACCTGCGTGGTGGTGG |
| | ACGTGAGCCAGGAAGATCCCGAGGTCCAGTTCAATTGGTACGT |
| | GGACGGCGTGGAAGTGCACAACGCCAAGACCAAGCCCAGAGAG |
| | GAACAGTTCCAGAGCACCTACCGGGTGGTGTCTGTGCTGACCGT |
| | GCTGCACCAGGACTGGCTGAACGGCAAAGAATACAAGTGCAAG |
| | GTGTCCAACAAGGGCCTGCCCAGCAGCATCGAAAAGACCATCA |
| | GCAAGGCCAAGGGCCAGCCTCGCGAGCCCCAGGTGTACACCCT |
| | GCCTCCCTCCCAGGAAGAGATGACCAAGAACCAGGTGTCCCTG |
| | ACCTGCCTGGTGAAGGGCTTCTACCCCAGCGACATCGCCGTGGA |
| | GTGGGAGAGCAACGGCCAGCCTGAGAACAACTACAAGACCACC |
| | CCTCCCGTGCTGGACAGCGACGGCAGCTTCTTCCTGTACAGCCG |
| | GCTGACCGTGGACAAGAGCCGGTGGCAGGAAGGCAACGTCTTT |
| | AGCTGCAGCGTGATGCACGAGGCCCTGCACAACCACTACACCC |
| | AGAAGAGCCTGAGCCTGTCCCTGGGCAAGATGTTCTGGGTGCTG |
| | GTGGTGGTGGGCGGGGTGCTGGCCTGCTACAGCCTGCTGGTGAC |
| | AGTGGCCTTCATCATCTTTTGGGTGAAACGGGGCAGAAAGAAA |
| | CTCCTGTATATATTCAAACAACCATTTATGAGACCAGTACAAAC |
| | TACTCAAGAGGAAGATGGCTGTAGCTGCCGATTTCCAGAAGAA |
| | GAAGAAGGAGGATGTGAACTGCGGGTGAAGTTCAGCAGAAGCG |
| | CCGACGCCCCTGCCTACCAGCAGGGCCAGAATCAGCTGTACAA |
| | CGAGCTGAACCTGGGCAGAAGGGAAGAGTACGACGTCCTGGAT |
| | AAGCGGAGAGGCCGGGACCCTGAGATGGGCGGCAAGCCTCGGC |
| | GGAAGAACCCCCAGGAAGGCCTGTATAACGAACTGCAGAAAGA |
| | CAAGATGGCCGAGGCCTACAGCGAGATCGGCATGAAGGGCGAG |
| | CGGAGGCGGGGCAAGGGCCACGACGGCCTGTATCAGGGCCTGT |
| | CCACCGCCACCAAGGATACCTACGACGCCCTGCACATGCAGGC |
| | CCTGCCCCCAAGGCTCGAGGGCGGCGGAGAGGGCAGAGGAAGT |
| | CTTCTAACATGCGGTGACGTGGAGGAGAATCCCGGCCCTAGGA |
| | TGCTTCTCCTGGTGACAAGCCTTCTGCTCTGTGAGTTACCACACC |
| | CAGCATTCCTCCTGATCCCACGCAAAGTGTGTAACGGAATAGGT |
| | ATTGGTGAATTTAAAGACTCACTCTCCATAAATGCTACGAATAT |
| | TAAACACTTCAAAAACTGCACCTCCATCAGTGGCGATCTCCACA |
| | TCCTGCCGGTGGCATTTAGGGGTGACTCCTTCACACATACTCCT |
| | CCTCTGGATCCACAGGAACTGGATATTCTGAAAACCGTAAAGG |
| | AAATCACAGGGTTTTTGCTGATTCAGGCTTGGCCTGAAAACAGG |
| | ACGGACCTCCATGCCTTTGAGAACCTAGAAATCATACGCGGCAG |
| | GACCAAGCAACATGGTCAGTTTTCTCTTGCAGTCGTCAGCCTGA |
| | ACATAACATCCTTGGGATTACGCTCCCTCAAGGAGATAAGTGAT |
| | GGAGATGTGATAATTTCAGGAAACAAAAATTTGTGCTATGCAA |
| | ATACAATAAACTGGAAAAAACTGTTTGGGACCTCCGGTCAGAA |
| | AACCAAAATTATAAGCAACAGAGGTGAAAACAGCTGCAAGGCC |
| | ACAGGCCAGGTCTGCCATGCCTTGTGCTCCCCCGAGGGCTGCTG |
| | GGGCCCGGAGCCCAGGGACTGCGTCTCTTGCCGGAATGTCAGCC |
| | GAGGCAGGGAATGCGTGGACAAGTGCAACCTTCTGGAGGGTGA |
| | GCCAAGGGAGTTTGTGGAGAACTCTGAGTGCATACAGTGCCAC |
| | CCAGAGTGCCTGCCTCAGGCCATGAACATCACCTGCACAGGAC |
| | GGGGACCAGACAACTGTATCCAGTGTGCCCACTACATTGACGGC |
| | CCCCACTGCGTCAAGACCTGCCCGGCAGGAGTCATGGGAGAAA |
| | ACAACACCCTGGTCTGGAAGTACGCAGACGCCGGCCATGTGTG |
| | CCACCTGTGCCATCCAAACTGCACCTACGGATGCACTGGGCCAG |
| | GTCTTGAAGGCTGTCCAACGAATGGGCCTAAGATCCCGTCCATC |
| | GCCACTGGGATGGTGGGGGCCCTCCTCTTGCTGCTGGTGGTGGC |
| | CCTGGGGATCGGCCTCTTCATGTGA |
| SEQ ID | MLLLVTSLLLCELPHPAFLLIPSVLTQPSSVSAAPGQKVTISCSGSTS |
| NO: 05 | NIGNNYVSWYQQHPGKAPKLMIYDVSKRPSGVPDRFSGSKSGNSA |
| E2 anti- | SLDISGLQSEDEADYYCAAWDDSLSEFLFGTGTKLTVLGGGGGSG |
| fluorescein | GGGSGGGGSQVQLVESGGNLVQPGGSLRLSCAASGFTFGSFSMSW |
| CAR | VRQAPGGGLEWVAGLSARSSLTHYADSVKGRFTISRDNAKNSVYL |
| The internal | QMNSLRVEDTAVYYCARRSYDSSGYWGHFYSYMDVWGQGTLVT |
| sequence | VSESKYGPPCPPCPAPEFDGGPSVFLFPPKPKDTLMISRTPEVTCVV |
| beginning at | VDVSQEDPEVQFNWYVDGVEVHNAKTKPREEQFQSTYRVVSVLT |
| ‘S’ to ‘G’ | VLHQDWLNGKEYKCKVSNKGLPSSIEKTISKAKGQPREPQVYTLPP |
| (both | SQEEMTKNQVSLTCLVKGFYPSDIAVEWESNGQPENNYKTTPPVL |
| underlined | DSDGSFFLYSRLTVDKSRWQEGNVFSCSVMHEALHNHYTQKSLSL |
| and bold) | SLGKMFWVLVVVGGVLACYSLLVTVAFIIFWVKRGRKKLLYIFKQ |
| comprises | PFMRPVQTTQEEDGCSCRFPEEEEGGCELRVKFSRSADAPAYQQG |
| the expressed | QNQLYNELNLGRREEYDVLDKRRGRDPEMGGKPRRKNPQEGLYN |
| CAR. | ELQKDKMAEAYSEIGMKGERRRGKGHDGLYQGLSTATKDTYDAL |
| | HMQALPPRLEGGGEGRGSLLTCGDVEENPGPRMLLLVTSLLLCELP |
| | HPAFLLIPRKVCNGIGIGEFKDSLSINATNIKHFKNCTSISGDLHILPV |
| | AFRGDSFTHTPPLDPQELDILKTVKEITGFLLIQAWPENRTDLHAFE |
| | NLEIIRGRTKQHGQFSLAVVSLNITSLGLRSLKEISDGDVIISGNKNL |
| | CYANTINWKKLFGTSGQKTKIISNRGENSCKATGQVCHALCSPEGC |
| | WGPEPRDCVSCRNVSRGRECVDKCNLLEGEPREFVENSECIQCHPE |
| | CLPQAMNITCTGRGPDNCIQCAHYIDGPHCVKTCPAGVMGENNTL |
| | VWKYADAGHVCHLCHPNCTYGCTGPGLEGCPTNGPKIPSIATGMV |
| | GALLLLLVVALGIGLFM |
| SEQ ID | ATGCTTCTCCTGGTGACAAGCCTTCTGCTCTGTGAGTTACCACAC |
| NO: 06 | CCAGCATTCCTCCTGATCCCAGACGTTGTAATGACCCAGACCCC |
| 4M5.3-CAR | TCTGTCTCTCCCCGTAAGCTTGGGCGACCAGGCGAGCATCTCTT |
| The initial | GTCGGTCTTCCCAGTCCCTGGTCCATTCAAACGGCAATACTTAC |
| start codon is | TTGCGGTGGTACTTGCAGAAGCCCGGTCAATCCCCAAAAGTGCT |
| underlined | GATATACAAGGTTAGCAATCGGGTCAGTGGAGTGCCCGACCGC |
| and begins at | TTCAGCGGAAGCGGATCCGGGACTGACTTCACTCTGAAGATCAA |
| nucleotide 1; | CCGGGTAGAAGCTGAAGACCTGGGGGTGTACTTCTGCTCTCAGT |
| the last | CAACACACGTGCCATGGACCTTTGGAGGTGGCACCAAGCTGGA |
| nucleotide | AATCAAATCATCAGCGGACGATGCCAAAAAAGACGCGGCCAAG |
| includes a | AAGGACGATGCCAAGAAGGATGATGCTAAAAAGGATGGCGGA |
| nucleotide of | GTCAAATTGGACGAGACAGGCGGGGGACTGGTGCAGCCCGGCG |
| the stop | GTGCCATGAAACTGTCTTGTGTGACCAGCGGCTTTACCTTCGGG |
| codon. | CATTATTGGATGAACTGGGTGCGACAGTCTCCAGAGAAAGGGC |
| The internal | TCGAGTGGGTGGCCCAGTTTCGAAATAAACCGTACAATTATGAG |
| sequence | ACCTACTATTCAGATTCTGTGAAAGGGCGCTTCACTATTTCACG |
| beginning at | CGACGACAGCAAAAGTTCCGTCTACCTTCAGATGAACAACCTTA |
| ‘GAC’ to | GAGTGGAGGATACCGGAATATACTACTGCACGGGTGCCAGTTA |
| ‘GGC’ (both | TGGCATGGAGTACTTGGGGCAGGGGACATCTGTGACCGTTTCTG |
| underlined | AGAGCAAGTACGGACCGCCCTGCCCCCCTTGCCCTGCCCCCGAG |
| and bold) | TTCGACGGCGGACCCAGCGTGTTCCTGTTCCCCCCCAAGCCCAA |
| encodes the | GGACACCCTGATGATCAGCCGGACCCCCGAGGTGACCTGCGTG |
| expressed | GTGGTGGACGTGAGCCAGGAAGATCCCGAGGTCCAGTTCAATT |
| CAR. | GGTACGTGGACGGCGTGGAAGTGCACAACGCCAAGACCAAGCC |
| | CAGAGAGGAACAGTTCCAGAGCACCTACCGGGTGGTGTCTGTG |
| | CTGACCGTGCTGCACCAGGACTGGCTGAACGGCAAAGAATACA |
| | AGTGCAAGGTGTCCAACAAGGGCCTGCCCAGCAGCATCGAAAA |
| | GACCATCAGCAAGGCCAAGGGCCAGCCTCGCGAGCCCCAGGTG |
| | TACACCCTGCCTCCCTCCCAGGAAGAGATGACCAAGAACCAGG |
| | TGTCCCTGACCTGCCTGGTGAAGGGCTTCTACCCCAGCGACATC |
| | GCCGTGGAGTGGGAGAGCAACGGCCAGCCTGAGAACAACTACA |
| | AGACCACCCCTCCCGTGCTGGACAGCGACGGCAGCTTCTTCCTG |
| | TACAGCCGGCTGACCGTGGACAAGAGCCGGTGGCAGGAAGGCA |
| | ACGTCTTTAGCTGCAGCGTGATGCACGAGGCCCTGCACAACCAC |
| | TACACCCAGAAGAGCCTGAGCCTGTCCCTGGGCAAGATGTTCTG |
| | GGTGCTGGTGGTGGTGGGGGGGGTGCTGGCCTGCTACAGCCTGC |
| | TGGTGACAGTGGCCTTCATCATCTTTTGGGTGAAACGGGGCAGA |
| | AAGAAACTCCTGTATATATTCAAACAACCATTTATGAGACCAGT |
| | ACAAACTACTCAAGAGGAAGATGGCTGTAGCTGCCGATTTCCA |
| | GAAGAAGAAGAAGGAGGATGTGAACTGCGGGTGAAGTTCAGCA |
| | GAAGCGCCGACGCCCCTGCCTACCAGCAGGGCCAGAATCAGCT |
| | GTACAACGAGCTGAACCTGGGCAGAAGGGAAGAGTACGACGTC |
| | CTGGATAAGCGGAGAGGCCGGGACCCTGAGATGGGCGGCAAGC |
| | CTCGGCGGAAGAACCCCCAGGAAGGCCTGTATAACGAACTGCA |
| | GAAAGACAAGATGGCCGAGGCCTACAGCGAGATCGGCATGAAG |
| | GGCGAGCGGAGGCGGGGCAAGGGCCACGACGGCCTGTATCAGG |
| | GCCTGTCCACCGCCACCAAGGATACCTACGACGCCCTGCACATG |
| | CAGGCCCTGCCCCCAAGGCTCGAGGGCGGCGGAGAGGGCAGAG |
| | GAAGTCTTCTAACATGCGGTGACGTGGAGGAGAATCCCGGCCC |
| | TAGGATGCTTCTCCTGGTGACAAGCCTTCTGCTCTGTGAGTTACC |
| | ACACCCAGCATTCCTCCTGATCCCACGCAAAGTGTGTAACGGAA |
| | TAGGTATTGGTGAATTTAAAGACTCACTCTCCATAAATGCTACG |
| | AATATTAAACACTTCAAAAACTGCACCTCCATCAGTGGCGATCT |
| | CCACATCCTGCCGGTGGCATTTAGGGGTGACTCCTTCACACATA |
| | CTCCTCCTCTGGATCCACAGGAACTGGATATTCTGAAAACCGTA |
| | AAGGAAATCACAGGGTTTTTGCTGATTCAGGCTTGGCCTGAAAA |
| | CAGGACGGACCTCCATGCCTTTGAGAACCTAGAAATCATACGCG |
| | GCAGGACCAAGCAACATGGTCAGTTTTCTCTTGCAGTCGTCAGC |
| | CTGAACATAACATCCTTGGGATTACGCTCCCTCAAGGAGATAAG |
| | TGATGGAGATGTGATAATTTCAGGAAACAAAAATTTGTGCTATG |
| | CAAATACAATAAACTGGAAAAAACTGTTTGGGACCTCCGGTCA |
| | GAAAACCAAAATTATAAGCAACAGAGGTGAAAACAGCTGCAAG |
| | GCCACAGGCCAGGTCTGCCATGCCTTGTGCTCCCCCGAGGGCTG |
| | CTGGGGCCCGGAGCCCAGGGACTGCGTCTCTTGCCGGAATGTCA |
| | GCCGAGGCAGGGAATGCGTGGACAAGTGCAACCTTCTGGAGGG |
| | TGAGCCAAGGGAGTTTGTGGAGAACTCTGAGTGCATACAGTGC |
| | CACCCAGAGTGCCTGCCTCAGGCCATGAACATCACCTGCACAGG |
| | ACGGGGACCAGACAACTGTATCCAGTGTGCCCACTACATTGACG |
| | GCCCCCACTGCGTCAAGACCTGCCCGGCAGGAGTCATGGGAGA |
| | AAACAACACCCTGGTCTGGAAGTACGCAGACGCCGGCCATGTG |
| | TGCCACCTGTGCCATCCAAACTGCACCTACGGATGCACTGGGCC |
| | AGGTCTTGAAGGCTGTCCAACGAATGGGCCTAAGATCCCGTCCA |
| | TCGCCACTGGGATGGTGGGGGCCCTCCTCTTGCTGCTGGTGGTG |
| | GCCCTGGGGATCGGCCTCTTCATGTGA |
| SEQ ID | MLLLVTSLLLCELPHPAFLLIPDVVMTQTPLSLPVSLGDQASISCRSS |
| NO: 07 | QSLVHSNGNTYLRWYLQKPGQSPKVLIYKVSNRVSGVPDRFSGSG |
| 4M5.3-CAR | SGTDFTLKINRVEAEDLGVYFCSQSTHVPWTFGGGTKLEIKSSADD |
| The internal | AKKDAAKKDDAKKDDAKKDGGVKLDETGGGLVQPGGAMKLSC |
| sequence | VTSGFTFGHYWMNWVRQSPEKGLEWVAQFRNKPYNYETYYSDS |
| beginning at | VKGRFTISRDDSKSSVYLQMNNLRVEDTGIYYCTGASYGMEYLGQ |
| ‘D’ to ‘G’ | GTSVTVSESKYGPPCPPCPAPEFDGGPSVFLFPPKPKDTLMISRTPEV |
| (both | TCVVVDVSQEDPEVQFNWYVDGVEVHNAKTKPREEQFQSTYRVV |
| underlined | SVLTVLHQDWLNGKEYKCKVSNKGLPSSIEKTISKAKGQPREPQV |
| and bold) | YTLPPSQEEMTKNQVSLTCLVKGFYPSDIAVEWESNGQPENNYKT |
| comprises | TPPVLDSDGSFFLYSRLTVDKSRWQEGNVFSCSVMHEALHNHYTQ |
| the expressed | KSLSLSLGKMFWVLVVVGGVLACYSLLVTVAFIIFWVKRGRKKLL |
| CAR. | YIFKQPFMRPVQTTQEEDGCSCRFPEEEEGGCELRVKFSRSADAPA |
| | YQQGQNQLYNELNLGRREEYDVLDKRRGRDPEMGGKPRRKNPQE |
| | GLYNELQKDKMAEAYSEIGMKGERRRGKGHDGLYQGLSTATKDT |
| | YDALHMQALPPRLEGGGEGRGSLLTCGDVEENPGPRMLLLVTSLL |
| | LCELPHPAFLLIPRKVCNGIGIGEFKDSLSINATNIKHFKNCTSISGDL |
| | HILPVAFRGDSFTHTPPLDPQELDILKTVKEITGFLLIQAWPENRTDL |
| | HAFENLEIIRGRTKQHGQFSLAVVSLNITSLGLRSLKEISDGDVIISG |
| | NKNLCYANTINWKKLFGTSGQKTKIISNRGENSCKATGQVCHALC |
| | SPEGCWGPEPRDCVSCRNVSRGRECVDKCNLLEGEPREFVENSECI |
| | QCHPECLPQAMNITCTGRGPDNCIQCAHYIDGPHCVKTCPAGVMG |
| | ENNTLVWKYADAGHVCHLCHPNCTYGCTGPGLEGCPTNGPKIPSI |
| | ATGMVGALLLLLVVALGIGLFM |
| SEQ ID | AGCGTGCTGACACAGCCTAGCTCCGTGTCTGCCGCCCCTGGCCA |
| NO: 08 | GAAAGTGACCATCAGCTGTAGCGGCAGCACCAGCAACATCGGC |
| A sequence | AACAACTACGTGTCCTGGTATCAGCAGCACCCCGGCAAGGCCCC |
| within SEQ | CAAGCTGATGATCTACGACGTGTCCAAGCGGCCCAGCGGCGTG |
| ID NO: 04 | CCCGATAGATTTTCCGGCAGCAAGAGCGGCAACAGCGCCAGCC |
| | TGGATATCAGCGGCCTGCAGTCTGAGGACGAGGCCGACTACTAT |
| | TGCGCCGCCTGGGACGATAGCCTGAGCGAGTTCCTGTTTGGCAC |
| | CGGCACCAAGCTGACAGTGCTGGGCGGAGGCGGAGGATCTGGC |
| | GGCGGAGGAAGTGGCGGAGGGGGATCTCAGGTGCAGCTGGTGG |
| | AAAGCGGCGGCAACCTGGTGCAGCCTGGCGGATCTCTGAGACT |
| | GAGCTGTGCCGCCAGCGGCTTCACCTTCGGCAGCTTCAGCATGA |
| | GCTGGGTGCGCCAGGCTCCTGGGGGAGGACTGGAATGGGTGGC |
| | AGGACTGAGCGCCAGAAGCAGCCTGACCCACTACGCCGATAGC |
| | GTGAAGGGCCGGTTCACCATCAGCCGGGACAACGCCAAGAACA |
| | GCGTGTACCTGCAGATGAACAGCCTGCGGGTGGAAGATACCGC |
| | CGTGTACTACTGCGCCAGACGGTCCTACGACAGCAGCGGCTACT |
| | GGGGCCACTTCTACAGCTACATGGACGTGTGGGGCCAGGGCAC |
| | CCTCGTGACAGTGTCTGAGAGCAAGTACGGACCGCCCTGCCCCC |
| | CTTGCCCTGCCCCCGAGTTCGACGGCGGACCCAGCGTGTTCCTG |
| | TTCCCCCCCAAGCCCAAGGACACCCTGATGATCAGCCGGACCCC |
| | CGAGGTGACCTGCGTGGTGGTGGACGTGAGCCAGGAAGATCCC |
| | GAGGTCCAGTTCAATTGGTACGTGGACGGCGTGGAAGTGCACA |
| | ACGCCAAGACCAAGCCCAGAGAGGAACAGTTCCAGAGCACCTA |
| | CCGGGTGGTGTCTGTGCTGACCGTGCTGCACCAGGACTGGCTGA |
| | ACGGCAAAGAATACAAGTGCAAGGTGTCCAACAAGGGCCTGCC |
| | CAGCAGCATCGAAAAGACCATCAGCAAGGCCAAGGGCCAGCCT |
| | CGCGAGCCCCAGGTGTACACCCTGCCTCCCTCCCAGGAAGAGAT |
| | GACCAAGAACCAGGTGTCCCTGACCTGCCTGGTGAAGGGCTTCT |
| | ACCCCAGCGACATCGCCGTGGAGTGGGAGAGCAACGGCCAGCC |
| | TGAGAACAACTACAAGACCACCCCTCCCGTGCTGGACAGCGAC |
| | GGCAGCTTCTTCCTGTACAGCCGGCTGACCGTGGACAAGAGCCG |
| | GTGGCAGGAAGGCAACGTCTTTAGCTGCAGCGTGATGCACGAG |
| | GCCCTGCACAACCACTACACCCAGAAGAGCCTGAGCCTGTCCCT |
| | GGGCAAGATGTTCTGGGTGCTGGTGGTGGTGGGCGGGGTGCTG |
| | GCCTGCTACAGCCTGCTGGTGACAGTGGCCTTCATCATCTTTTG |
| | GGTGAAACGGGGCAGAAAGAAACTCCTGTATATATTCAAACAA |
| | CCATTTATGAGACCAGTACAAACTACTCAAGAGGAAGATGGCT |
| | GTAGCTGCCGATTTCCAGAAGAAGAAGAAGGAGGATGTGAACT |
| | GCGGGTGAAGTTCAGCAGAAGCGCCGACGCCCCTGCCTACCAG |
| | CAGGGCCAGAATCAGCTGTACAACGAGCTGAACCTGGGCAGAA |
| | GGGAAGAGTACGACGTCCTGGATAAGCGGAGAGGCCGGGACCC |
| | TGAGATGGGCGGCAAGCCTCGGCGGAAGAACCCCCAGGAAGGC |
| | CTGTATAACGAACTGCAGAAAGACAAGATGGCCGAGGCCTACA |
| | GCGAGATCGGCATGAAGGGCGAGCGGAGGCGGGGCAAGGGCC |
| | ACGACGGCCTGTATCAGGGCCTGTCCACCGCCACCAAGGATACC |
| | TACGACGCCCTGCACATGCAGGCCCTGCCCCCAAGGCTCGAGG |
| | GCGGCGGAGAGGGCAGAGGAAGTCTTCTAACATGCGGTGACGT |
| | GGAGGAGAATCCCGGC |

### EXAMPLES

### Example 1—Synthesis of FITC-Folate

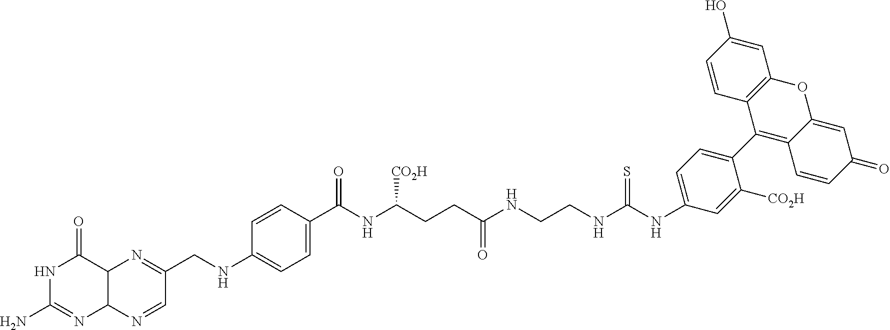 [SMILES: NC1=NC2N=CC(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)=NC2C(=O)N1]

Folate-γ-ethylenediamine was coupled to fluorescein isothiocyanate (FITC) isomer I (Sigma-Aldrich) in anhydrous dimethylsulfoxide (DMF) in the presence of tetramethylguanidine and diisopropylamine. The crude product was loaded onto an Xterra RP18 preparative HPLC column (Waters) and eluted with gradient conditions starting with 99% 5 mM sodium phosphate (mobile phase A, pH 7.4) and 1% acetonitrile (mobile phase B) and reaching 90% A and 10% B in 10 min at a flow rate of 20 mL/min. Under these conditions, the FITC-folate main peak typically eluted at 27-50 min. The quality of the FITC-folate fraction was monitored by analytical reverse-phase HPLC with a UV detector. Fractions with greater than 98.0% purity (LCMS) were lyophilized to obtain the final FITC-folate product. As known in the art, the compound with this structure is also referred to as EC17.

### Example 2—Synthesis of FITC-PEG12-Folate

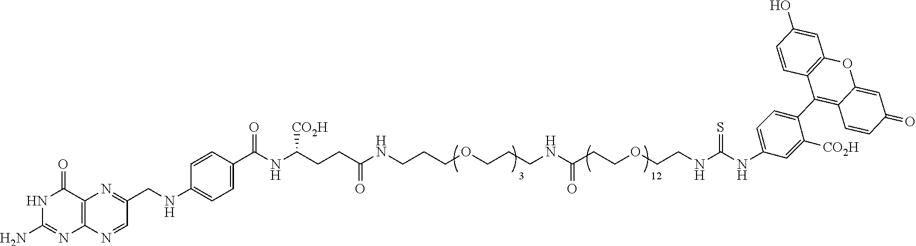

Universal polyethylene glycol (PEG) Nova Tag™ resin (0.2 g) was loaded into a peptide synthesis vessel and washed with isopropyl alcohol (i-PrOH) (3×10 mL) and dimethylformamide (DMF, 3×10 mL). 9-fluorenylmethoxycarbonyl (Fmoc) deprotection was carried out using 20% piperidine in DMF (3×10 mF). Kaiser tests were performed to assess reaction progress. To the vessel was then introduced a solution of Fmoc-F-glutamic acid 5-tert-butyl ester (Fmoc-Glu-(O-t-Bu)-OH) (23.5 mg) in DMF, N,N-diisopropylethylamine (z-Pr₂NEt) (4 equiv), and benzotriazol-1-yl-oxytripyrrolidinophosphonium hexafluorophosphate (PyBOP) (2 equiv). Fmoc deprotection was carried out using 20% piperidine in DMF (3×10 mF). To the vessel was then introduced a solution of N¹⁰-TFA-Pte-OH (22.5 mg), DMF, z-Pr₂NEt (4 equiv), and PyBOP (2 equiv). Argon was bubbled for 2 h, and the resin was washed with DMF (3×3 mF) and i-PrOH (3×3 mF). After swelling the resin in dichloromethane (DCM), a solution of 1M hydroxybenzotriazole (HOBT) in DCM/trifluoroethane (TFE) (1:1) (2×3 mF) was added. Argon was bubbled for 1 h, the solvent was removed, and the resin was washed with DMF (3×3 mF) and i-PrOH (3×3 mF). After swelling the resin in DMF, a solution of Fmoc-NH-(PEG)₁₂-COOH (46.3 mg) in DMF, z-Pr₂NEt (4 equiv), and PyBOP (2 equiv) was added. Argon was bubbled for 2 h, and the resin was washed with DMF (3×3 mF) and i-PrOH (3×3 mF). Fmoc deprotection was carried out using 20% piperidine in DMF (3×10 mF). Kaiser tests were performed to assess reaction progress. To the vessel was then introduced a solution of FITC (Fife Technologies 21.4 mg) in DMF and z-Pr₂NEt (4 equiv), then Argon was bubbled for 2 h, and the resin was washed with DMF (3×3 mL) and i-PrOH (3×3 mL). Then to the vessel was added 2% NH₂NH₂ in DMF (2×2 mL). The final compound was cleaved from the resin using a TFAiPPO: triisopropylsilane (TIS) (95:2.5:2.5) (Cleavage Solution) and concentrated under vacuum. The concentrated product was precipitated in Et₂O and dried under vacuum. The crude product was purified using preparative RP-HPLC (mobile phase: A=10 mM ammonium acetate pH=7, B=ACN; method: 0/o B to 30% B in 30 min at 13 mL/min). The pure fractions were pooled and freeze-dried, providing the FITC-PEG12-Folate.

### Example 3—Synthesis of FITC-PEG20-Folate

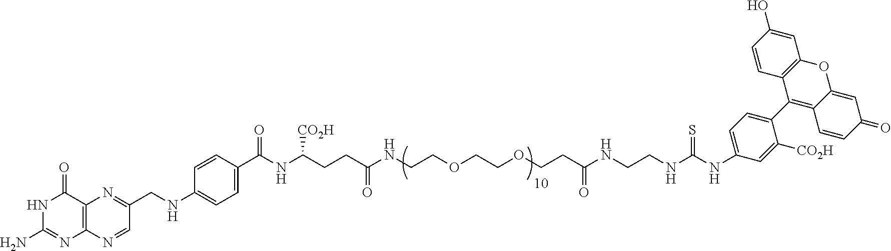

Ethylenediamine, polymer-bound (200-400 mesh)-resin (50 mg) was loaded into a peptide synthesis vessel and swollen with DCM (3 mL) followed by DMF (3 mL). To the vessel was then introduced the Fmoc-PEG20-COOH solution (131 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete the reaction with Fmoc-Glu-OtBu (72 mg, 2.0 equiv) and Tfa.Pteroic-acid (41 mg, 1.2 equiv) coupling steps. The resin was washed with 2% hydrazine in DMF 3×10 mL (5 min) to cleave the trifluoro-acetyl protecting group on pteroic acid and washed with i-PrOH (3×10 mL) followed by DMF (3×10 mL). The resin was dried under argon for 30 min. The folate-peptide was cleaved from the resin using the Cleavage Solution. 10 mL of the cleavage mixture was introduced and argon was bubbled for 1.5 h. The cleavage mixture was drained into a clean flask. The resin was washed 3 times with more cleavage mixture. The combined mixture was concentrated under reduced pressure to a smaller volume (~5 mL) and precipitated in ethyl ether.

The precipitate was collected by centrifugation, washed with ethyl ether (3 times) and dried under high vacuum. The dried Folate-PEC₂₀-EDA (1.0 equiv) was treated with FITC (50 mg, 1.5 equiv) in DMSO and DIPEA at room temperature. Progress of the reaction monitored by LCMS. After 8 h the starting material was consumed to give the product. The crude reaction mixture was purified by preparative HPLC, (mobile phase A=10 mM Ammonium Acetate, pH=7; Organic phase B=Acetonitrile; Method: 0% B to 30% B in 35 minutes at 13 mL/min) and provided FITC-PEG20-Folate in 60% yield.

### Example 4—Synthesis of FITC-PEG108-Folate

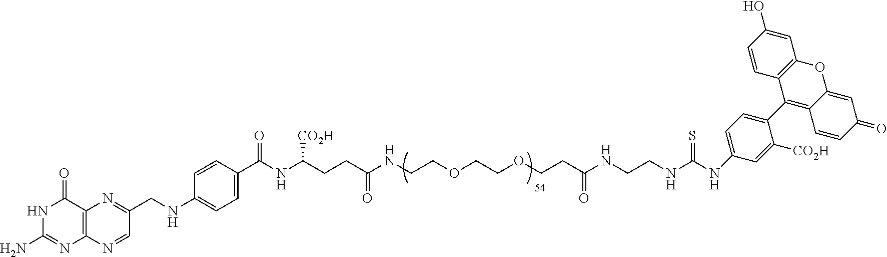

Ethylenediamine, polymer-bound (200-400 mesh)-resin (50 mg) was loaded in a peptide synthesis vessel and swollen with DCM (3 mL) followed by DMF (3 mL). To the vessel was then introduced the Fmoc-PEC₃₆-COOH solution (161 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete reaction with 2× Fmoc-PEG₃₆-COOH (161 mg, 1.0 equiv), Fmoc-Glu-OtBu (72 mg, 2.0 equiv) and Tfa.Pteroic-acid (41.0 mg, 1.2 equiv) coupling steps. At the end the resin was washed with 2% hydrazine in DMF 3×10 mL (5 min) to cleave the trifluoro-acetyl protecting group on pteroic acid and washed with i-PrOH (3×10 mL) followed by DMF (3×10 mL). The resin was dried under argon for 30 min. Folate-peptide was cleaved from the resin using the Cleavage Solution. 10 mL of the cleavage mixture was introduced and argon was bubbled for 1.5 h. The cleavage mixture was drained into a clean flask. The resin was washed 3× with more Cleavage Solution. The combined mixture was concentrated under reduced pressure to a smaller volume (~5 mL) and precipitated in ethyl ether.

The precipitate was collected by centrifugation, washed with ethyl ether (3×) and dried under high vacuum. The dried Folate-PEG108-EDA (1.0 equiv) was treated with FITC (50 mg, 1.5 equiv) in DMSO and DIPEA at room temperature. Reaction progress was monitored by LCMS. After 10 h starting material was consumed to give the product. The crude reaction mixture was purified by preparative HPLC, (mobile phase A=10 mM Ammonium Acetate, pH=7; Organic phase B=Acetonitrile; Method: 0% B to 30% B in 35 minutes at 13 mL/min) and provided FITC-PEG108-Folate in 64% yield.

### Example 5—Synthesis of FITC-DUPA

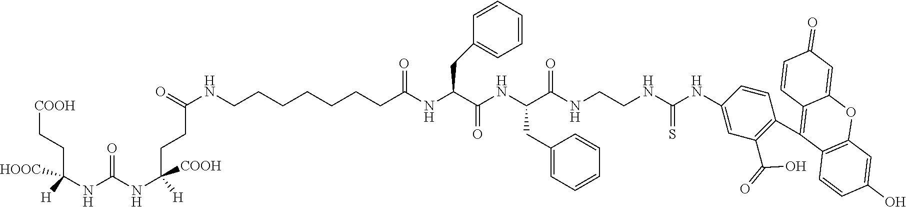 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

DUPA-FITC was synthesized by solid phase methodology as follows. Universal Nova Tag™ resin (50 mg, 0.53 mM) was swollen with dichloromethane (DCM, 3 mL) followed by dimethylformamide (DMF, 3 mL). A solution of 20% piperidine in DMF (3×3 mL) was added to the resin, and argon was bubbled for 5 min. The resin was washed with DMF (3×3 mL) and isopropyl alcohol (i-PrOH, 3×3 mL). After swelling the resin in DMF, a solution of DUPA-(OtBu)-OH (1.5 equiv), HATU (2.5 equiv), and z-Pr2NEt (4.0 equiv) in DMF was added. Argon was bubbled for 2 h, and resin was washed with DMF (3×3 mL) and i-PrOH (3×3 mL). After swelling the resin in DCM, a solution of 1 M HOBt in DCM/TFE (1:1) (2-3 mL) was added. Argon was bubbled for 1 h, the solvent was removed, and resin was washed with DMF (3×3 mL) and i-PrOH (3×3 mL). After swelling the resin in DMF, a solution of Fmoc-Phe-OH (2.5 equiv), HATU (2.5 equiv) and DIPEA (4.0 equiv) in DMF was added. Argon was bubbled for 2 h, and the resin was washed with DMF (3×3 mL) and i-PrOH (3×3 mL). The above sequence was repeated for 2 more coupling steps for addition of 8-aminooctanoic acid and fluorescein isothiocyanate or rhodamine B isothiocyanate. The final compound was cleaved from the resin using the Cleavage Solution and concentrated under vacuum. The concentrated product was precipitated in diethyl ether and dried under vacuum. The crude product was purified using preparative RP-HPLC [λ=488 nm; solvent gradient: 1% B to 80% B in 25 min, 80% B wash 30 min run; A=10 mM NH4OAC, pH=7; B=acetonitrile (ACN)]. ACN was removed under vacuum, and purified fractions were freeze-dried to yield FITC-DUPA as a brownish-orange solid. RP-HPLC: tR=8.0 min (A=10 mM NH4OAc, pH=7.0. B=ACN, solvent gradient: 1% B to 50% B in 10 min, 80% B wash 15 min ran). 1H NMR (DMSO-d6/D2O): δ 0.98-1.27 (ms, 9H); 1.45 (b, 3H); 1.68-1.85 (ms, 11H); 2.03 (m, 8H); 2.6-3.44 (ms, 12H); 3.82 (b, 2H); 4.35 (m, 1H); 6.53 (d, J=8.1 Hz, 2H), 6.61 (dd, J=5.3, 3.5 Hz, 2H); 6.64 (s, 2H); 7.05 (d, J=8.2 Hz, 2H), 7.19 (m, 5H); 7.76 (d, J=8.0 Hz, 1H); 8.38 (s, 1H). HRMS (ESI) (m/z): (M+H)+ calcd for C51H59N7O15S, 1040.3712, found, 1040.3702. UV/vis: λ max=491 nm.

### Example 6—Synthesis of FITC-PEG12-DUPA

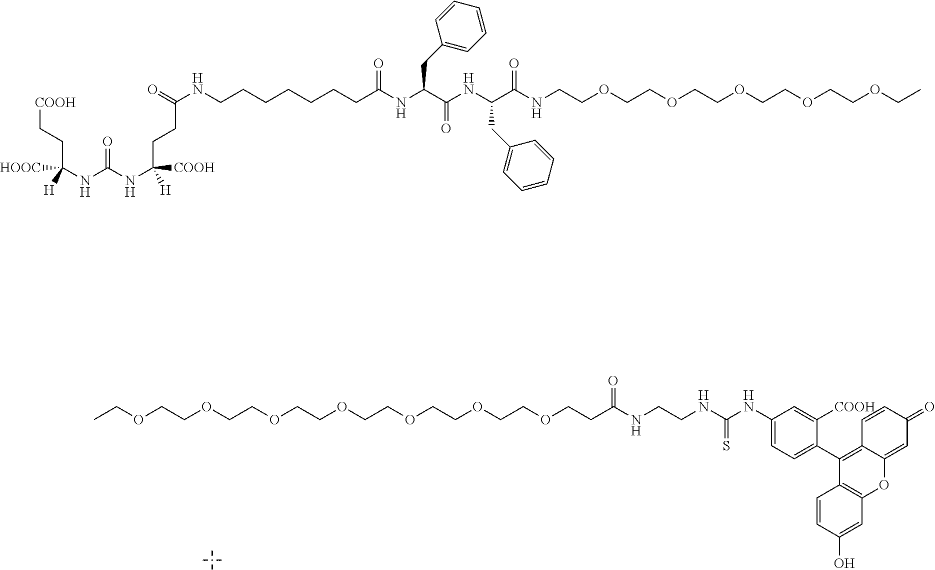

1,2-Diaminoethane trityl-resin (0.025 g) was loaded into a peptide synthesis vessel and washed with i-PrOH (3×10 mL), followed by DMF (3×10 mL). To the vessel was then introduced a solution of Fmoc-NH-(PEG)₁₂-COOH (42.8 mg) in DMF, z-Pr₂NEt (2.5 equiv), and PyBOP (2.5 equiv). The resulting solution was bubbled with Ar for 1 h, the coupling solution was drained, and the resin washed with DMF (3×10 mL) and i-PrOH (3×10 mL). Kaiser tests were performed to assess reaction progress. Fmoc deprotection was carried out using 20% piperidine in DMF (3×10 mL). This procedure was repeated to complete the all coupling steps (2×1.5 equiv of Fmoc-Phe-OH and 1.5 equiv of 8-aminooctanoic acid and 1.2 equiv of DUPA were used on each of their respective coupling steps). After the DUPA coupling, the resin was washed with DMF (3×10 mL) and i-PrOH (3×10 mL) and dried under reduced pressure. The peptide was cleaved from the resin in the peptide synthesis vessel using the Cleavage Solution. 15 mL of the Cleavage Solution was added to the peptide synthesis vessel, and the reaction was bubbled under Ar for 15 min. The resin was treated with two additional 10 mL quantities of the Cleavage Solution for 5 min each. The cleavage mixture was concentrated to about 5 mL and precipitated with ethyl ether. The precipitate was collected by centrifugation, washed with ethyl ether (3×), and dried under high vacuum, resulting in the recovery of crude material. To a stirred solution of the crude DUPA-(PEG)₁₂-EDA (10 mg) and FITC (5.6 mg) in dimethylsulfoxide (DMSO, 1 mL) was added z-Pr₂NEt (5 equiv) at room temperature and stirred for 6 h under argon. The reaction was monitored by LCMS and purified by preparative HPLC (mobile phase: A=10 mM ammonium acetate pH=7, B=ACN; method: 0% B to 50% B in 30 min at 13 mL/min). The purified fractions were pooled and freeze-dried, providing the FITC-PEG12-DUPA.

### Example 7—Synthesis of FITC-PEG11-NK1

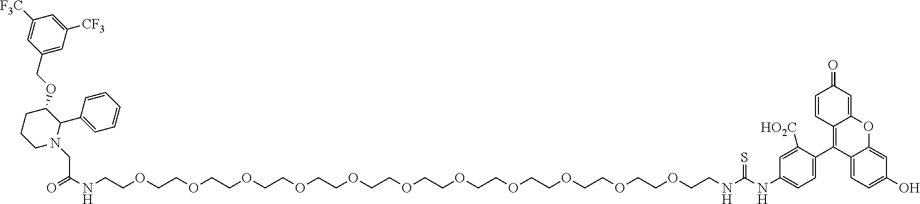 [SMILES: O=C(CN1CCC[C@H](OCc2cc(C(F)(F)F)cc(C(F)(F)F)c2)C1c1ccccc1)NCCOCCOCCOCCOCCOCCOCCOCCOCCOCCOCCOCCNC(=S)Nc1ccc(-c2c3ccc(=O)cc-3oc3cc(O)ccc23)c(C(=O)O)c1]

To a stirred solution of NK-1 (0.02 g, 0.0433 mmol, 1.0 eq.), O-(2-Aminoethyl)-O′-[2-(Boc-amino)ethyl |decaethylene glycol (BocNH-PEG₁₁-NH₂) (Sigma, 0.0336 g, 0.0521 mmol, 1.2 eq.), Benzotriazol-1-yl-oxytripyrrolidinophosphoniumhexafluorophosphate (PyBOP) (0.027 g, 0.0521 mmol, 1.2 eq.) in dry CH₂Cl₂ was added AW-Diisopropylethylamine (DIPEA) (0.076 mL, 0.4338 mmol, 10 eq.) under argon at room temperature. The reaction progress was monitored by LCMS and purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, 1=220 nm, 254 nm). The pure fractions were collected, all organic solvents were evaporated and the sample was lyophilized for 48 h to provide the NK1-PEG₁₁-NHBoc. Yield: 40.13 mg (97%). To the NK1-PEG₁₁-NHBoc (0.0165 g, 0.015 mmol) in dry DCM was added trifluoroacetic acid (TFA, 20 eq.) and the reaction mixture was stirred for 4 h at r.t. The excess TFA was removed, and the remaining solution was diluted with water and extracted using CH₂Cl₂ (3×5 mL). The combined organic layers were washed with brine, dried (Na₂SO₄) and concentrated. The residue obtained was dried under vacuum and used for the next-step without further purification. A stirred solution of NK1-PEG₁₁-NH₂ (0.008 g, 0.0081 mmol, 1.0 eq.), Fluorescein isothiocyanate (FITC) (Sigma, 0.0037 g, 0.0097 mmol, 1.2 eq.) in dry dimethylsulfoxide (DMSO, 0.3 mL) was added to diisopropylethyl amine (0.0028 mL, 0.0162 mmol, 2.0 eq.) at room temperature under argon.

The reaction progress was monitored by LCMS and the product was purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=280 nm). The pure fractions were collected, all organic solvents were evaporated and the sample was lyophilized for 48 h to provide the FITC-PEG11-NK1 in a yield of 8.54 mg (77%).

The NK-1 compound was synthesized by a two-step procedure starting from the base ligand, which was prepared by using a procedure in the literature (e.g., U.S. Pat. No. 11,141,494; incorporated herein by reference).

### Example 8—Synthesis of FITC-CA9

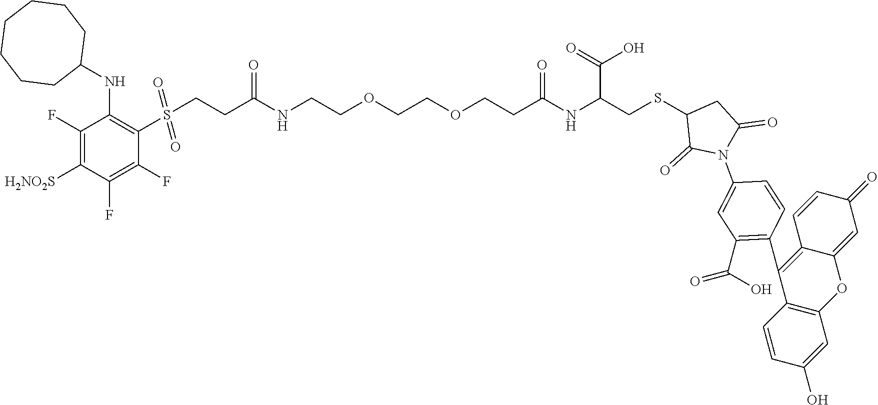 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

In a 50 mL round bottom flask CA9 ligand (53.6 mg, synthesized in lab) was dissolved in a desired amount of N,N-Dimethylformamide (DMF) (2-3 mL) using a Teflon magnetic stir bar. Ambient air was removed using vacuum and replaced with nitrogen gas, this was done in three cycles. Then the round bottom was kept under constant nitrogen gas. To the flask, 28.9 mg of N-(3-Dimethylaminopropyl)-N′-ethylcarbodiimide hydrochloride (EDC) was added followed by 21.6 mg 1-Hydroxybenzotriazole hydrate (HOBt) and 18.9 μL of Boc-PEG2-NH₂ (purchased from Sigma Aldrich). 5.4 μL of triethylamine (TEA) was added last and the reaction was allowed to stir overnight. The reaction mixture was purified using HPLC and confirm with UHPLC-MS (target m/z of 831). Acetonitrile was removed using high vacuum rotary evaporation and place on lyophilizer for 48 hours. Deprotection of Boc was done with 1:1 TFA:DCM for 30 minutes. TFA/DCM was removed using high vacuum rotary evaporation followed by 30 minutes on high vacuum. The compound was then dissolved in DMF and combined with 5 molar equivalents of N,N-Diisopropylethylamine (DIPEA). 16 mg of fluorescein isothiocyanate (purchased from Life Technologies) was added to the solution and stirred for 1 hour. Reaction mixture was purified by HPLC, and target compound was confirmed with UHPLC-MS (target m/z of 1120). The samples were placed on a lyophilizer for 48 hours and store compound at −20° C.

### Example 9—Synthesis of FITC-PEG2-CA9

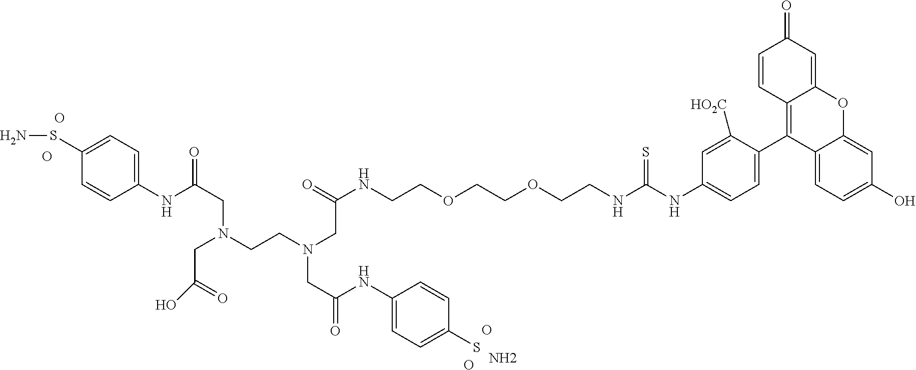

CA9 ligand (53.6 mg) was dissolved in DMF (2-3 mL) in a 50 mL round bottom flask using a Teflon magnetic stir bar. Ambient air was removed using a vacuum and replaced with nitrogen gas, this was done in three cycles. The round bottom flask was kept under constant nitrogen gas. To the flask, 28.9 mg of N-(3-Dimethylaminopropyl)-N′-ethylcarbodiimide hydrochloride (EDC) was added followed by 21.6 mg 1 -Hydroxybenzotriazole hydrate (HOBt) and 18.9 μL of Boc-PEG2-NH₂ (Sigma Aldrich). 5.4 μL of triethylamine (TEA) was added and the reaction was stirred overnight. The reaction mixture was purified using HPLC and confirmed with UHPLC-MS (target m/z of 831). Acetonitrile was removed using high vacuum rotary evaporation and the product lyophilized. The compound was mixed with 1:1 TFA:DCM for 30 minutes. The TFA/DCM was removed using high vacuum rotary evaporation followed by 30 minutes on high vacuum. The compound was then dissolved in DMF and combined with 5 molar equivalents of z-Pr₂NEt, 16 mg of fluorescein isothiocyanate (Life Technologies) and stirred for 1 h. The reaction mixture was purified by HPLC, and the target compound was confirmed with UHPLC-MS (target m/z of 1120). The samples were lyophilized and stored at −20° C.

### Example 10—T Cell Preparation

Human peripheral blood mononuclear cells (PBMCs) were isolated from whole blood of healthy donors by using Ficoll density gradient centrifugation (GE Healthcare Lifesciences). T cells were then isolated from PBMCs by using an EasySep™ Human T Cell Isolation Kit (STEM CELL technologies). T cells were cultured in TexMACS medium (Miltenyi Biotech Inc) with 40-100 TU/mL human IL-2 (Miltenyi Biotech), 2% human AB type serum, and 1% penicillin/streptomycin sulfate. Dynabeads Human T-Activator CD3/CD28 (ThermoFisher Scientific) were added to T cells at 1:1 ratio to activate T cells. 12-24 hours after activation, T cells were transduced with FITC-CAR lentiviral particles in the presence of 8 μg/mL polybrine (Santa Cruz Biotech) by spinfection at 1,200 g for 90 minutes at 22-32° C. T cell mixture containing those with CAR modification (CAR-Ts) and those without CAR modification (non-transformed Ts) was cultured in the presence of activation beads for 6 days before the removal of activation beads. Fluorescence-Activated Cell Sorting was used to sort out CAR-T cells (GFP positive) and non-transformed T cells (GFP negative) based on their GFP expression. The sorted T cells were cultured for 7-15 days before injection into mice. When a T cell mixture was used, CAR-T cells and non-transformed T cells were mixed at the desired ratio before mouse injection.

### Example 11—Generation of Lentiviral Vector Encoding CAR Gene

An overlap PCR method was used to generate CAR constructs comprising scFv against fluorescein. scFV against fluorescein, 4M5.3 (Kd=270 fM, 762 bp) derived from anti-fluorescein (4-4-20) antibody was synthesized. Sequence encoding the human CD8α signal peptide (SP, 63 bp), the hinge, and transmembrane region (249 bp), the cytoplasmic domain of 4-1BB (CD137, 141 bp) and the CD3ζ chain (336 bp), as shown in FIG. 2, were fused with the anti-fluorescein scFV by overlapping PCR. The resulting CAR construct (1551 bp) was inserted into EcoRI/NotI cleaved lentiviral expression vector pCDH-EF1-MCS-(PGK-GFP) (FIG. 2, System Biosciences). The sequence of the CAR construct in lentiviral vector was confirmed by DNA sequencing. An exemplary CAR nucleic acid coding sequence may comprise SEQ ID NO:01. In SEQ ID NO:01, the first ATG is the start codon. An exemplary CAR amino acid sequence may comprise SEQ ID NO:02. An exemplary CAR amino acid sequence insert may comprise SEQ ID NO:03. In SEQ ID NO:03, the first GCCACC sequence may comprise a restriction enzyme cleavage site, followed by the ATG start codon. The encoded amino acid sequence may comprise SEQ ID NO:02. More example sequences encoding CARs or portions thereof are listed in TABLE 1.

### Example 12—Production of Lentivirus Containing CAR Gene for Human T Cell Transduction

To prepare lentiviral virus containing an anti-fluorescein (i.e., anti-FITC) single chain fragment variable (scFv) CAR, a HEK-293TN packaging cell line was co-transfected with the lentiviral vector encoding anti-fluorescein scFv CAR and a 2nd generation of a lentiviral packaging plasmid mix (Cellecta) or ViraPower™ Lentiviral Packaging Mix (ThermoFisher). After 24 and 48 hours of transfection, supernatants containing the lentivirus with the CAR gene were harvested and virus particles were concentrated by the standard polyethylene glycol virus concentration method (Clontech®) for future transduction with human T cells.

### Example 13—Isolation of Human T Cells from Human PBMC

T cells were isolated from human peripheral blood mononuclear cells (PBMC) by Ficoll density gradient centrifugation (GE Healthcare Lifesciences). After washing away remaining Ficoll solution, T cells were isolated by using an EasySep™ Human T Cell Isolation Kit (STEM CELL technologies). Purified T cells were cultured in TexMACS™ medium (Miltenyi Biotech Inc) with 1% penicillin and streptomycin sulfate in the presence of human IL-2 (100 IU/mL, Miltenyi Biotech Inc). T cells were cultured at density of 1×10⁶ cells/mL in multi-well plates. T cells were split and re-feed every 2-3 days.

### Example 14—Transduction of Human T Cells

Isolated T cells were activated with Dynabeads coupled with anti-CD3/CD28 antibodies (Life Technologies) for 12-24 hours in the presence of human IL-2 (100 IU/mL), then transduced with lentivirus encoding an anti-fluorescein CAR gene. Cells were harvested after 72 hours and the expression of CAR on transduced T cells was identified by measuring GFP fluorescent cells using flow cytometry.

### Example 15—Human Osteosarcoma-Folate Receptor Alpha (HOS-FR-α) and AML Study schema

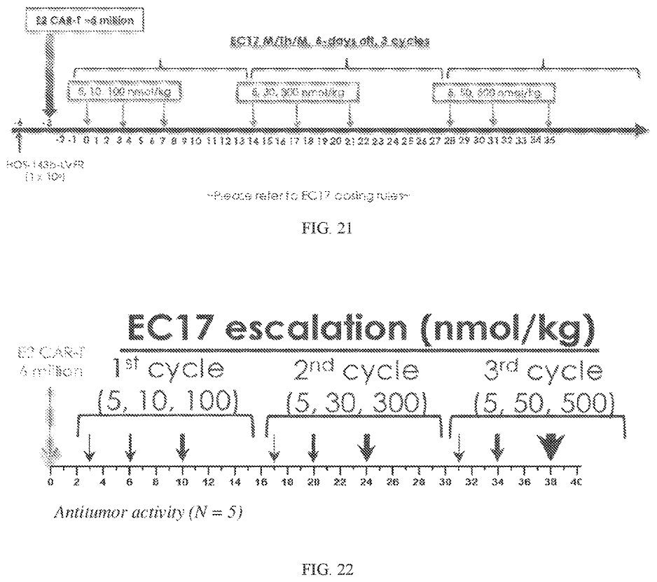

A dosing regimen is shown in FIG. 21 which includes administration of about 5 million E2 CAR T cells at day −3; administration of EC17 at days 0, 3, 7, 14, 17, 21, 28, 31 and 35. Sequential escalating doses of EC17 will be administered under the following rules: If no CRS or neurotoxicity, advance through EC17 dose escalation as planned. If CRS grade≤2, administer all subsequent EC17 doses at die Sequence level that provoked the non-serious CRS in the stone cycle. If CRS grade>2, cancel further EC17 doses in the current Cycle, and commence all subsequent Cycles at the next highest Sequence below that which provoked the severe CRS (sCRS).

### A. Tumor Implantation

HOS-FRα (i.e. HOS-143b-LV-FRα) is a subclone of HOS-143B (ATCC CRL-8303) stably transfected with human FRα. HOS-143B was originally derived from a 13-year-old Caucasian female with osteosarcoma. The tumor cells were grown in folate-deficient RPMI 1640 with 5% FBS at 37° C. in a 5% CO₂ humidified atmosphere. For the in-vivo study, tumor cells were inoculated subcutaneously at 1×10⁶ cells per animal.

### B. CAR-T Cell Administration

EGFRt-sorted anti-FITC E2 scFv-CAR T cells were frozen in a T-cell freezing medium. Upon arrival, vials of frozen CAR-T cells were immediately stored at −80° C. The CAR-T cells were quickly thawed at 37° C., washed twice with PBS, and used for animal injection at 6 million viable EGFRt+E2 CAR-T cells (CD4/CD8 at ~1:1) per animal. A small aliquot was taken on the day infusion for flow cytometric analysis of E2-CAR T-cell phenotypes.

### C. Preparation of Dosing Drug Solutions

EC17 dosing solutions were prepared when dosing began by weighing appropriate amounts of each compound, reconstituting in PBS, pH 7.4, sterile filtering the drug solution through a 0.22 pm PVDF syringe filter, and freezing aliquots for each day of dosing at −20° C.

### D. Compound Administration and Evaluation

All EC17 doses were given towards the end of day (3-4 PM) to allow potential CRS (cytokine release syndrome) to develop overnight. On the following morning, animals were scored according to the CRS grading system shown in TABLE 2.

TABLE 2
CRS Grading Scale

| 0 | 1 | 2 | 3 | 4 | 5 |
|---|---|---|---|---|---|
| Normal | ~pilo | pilo | pilo, greasy | moribund | Death |
| | ~greasy | greasy | bunched, thin | bunched, | |
| | still | less | not active, | pale | |
| | active | active | unless | lethargic | |
| | | | stimulated | | |

Tumor growth, body weight, and overall assessment: Tumor sizes were monitored 2-3 times/week and body weight measured 2-3 times/week. On the days immediately after any EC17 dose, body weight measurement was taken daily and attention was given to gross animal morphology and behavior.

Euthanasia: Euthanasia was performed when mice had lost ≥20% of their weight, when tumors reached ≥1500 mm³. Euthanasia was also performed if mice lost a lot of weight in a short duration (e.g., due to severe head-tilt), or when they approached moribund conditions per CRS grading system.

An EC17 intra-host dose escalation treatment (3 cycles) is depicted in FIG. 22.

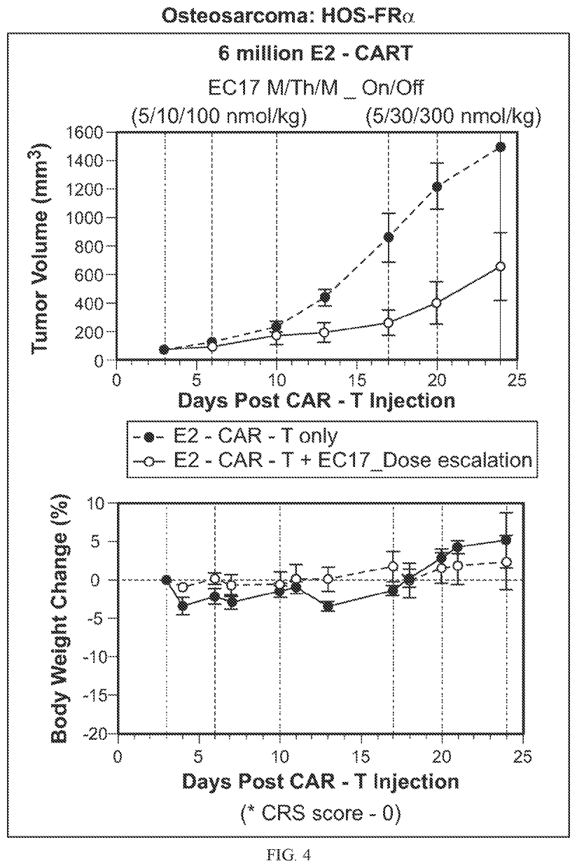

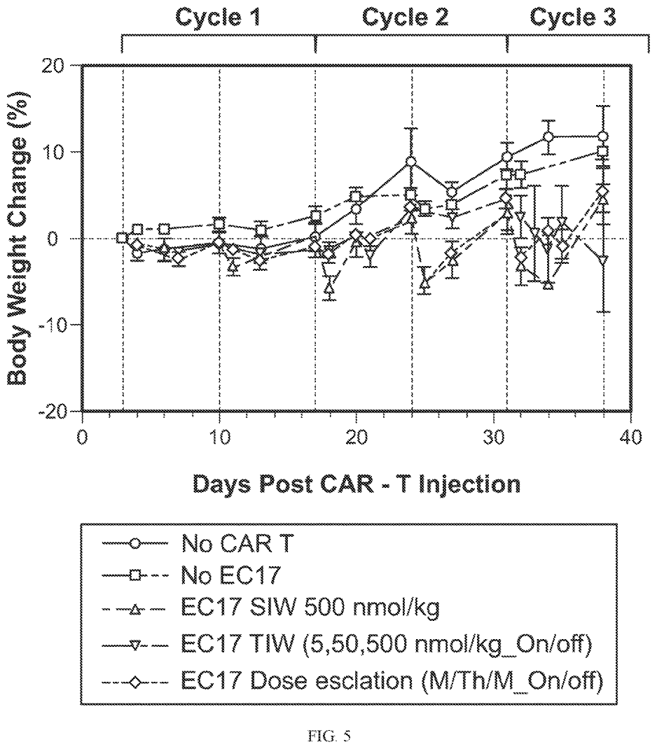

As seen in FIG. 4: Cohort 1 (E2-CAR T-cells only) showed HOS-FRα grew aggressively without EC17 treatment. Cohort 2 (EC17 5/100/1000 nmol/kg, M/Th/M) showed significant tumor regression without body weight loss or CRS through this study. The slow EC17 dose escalation (M/Th/M) regimen was safe. No body weight loss or symptoms of cytokine release syndrome (CRS) were observed in the HOS-FRα tumor-bearing mice.

One objective of this E2-CAR T-cell study was to test EC17/E2 CAR-T cell activity and related toxicity (e.g., sCRS) using multiple dosing regimens starting 3 days after CAR-T infusion. Three different EC17 dosing regimens include (a) once-a-week (SIW) at 500 nmol/kg, (b) “T1W-like” at 5, 50, and 500 nmol/kg on Monday/Wednesday/Friday with 9-day intervals, and (c) “T1W-like” at 5, 100, and 1000 nmol/kg on Monday/Thursday/Monday with 7-day intervals. Experimental groups are listed in TABLE 3.

TABLE 3

| | | | C1, Wk 1 | | C2, Wk 1 | | C3, Wk 1 | | N | |
|---|---|---|---|---|---|---|---|---|---|---|
| Cohort | Groups | T cells | (EC17) | | (EC17) | | (EC17) | | (efficacy) | N (satellite) |
| 1 | Tumor | N/A | N/A | | N/A | | N/A | | 5 | N/A |
| | only | | | | | | | | | |
| 2 | CAR-T | 6 | N/A | | N/A | | N/A | | 5 | 0/3/0/3/0/3/3 |
| | only | million | | | | | | | | |
| | | CAR-T | | | | | | | | |
| 3 | EC17 | 6 | 500 | nmol/kg | 500 | nmol/kg | 500 | nmol/kg | N/A | 3/3/0/3/0/3/3 |
| | S1W | million | | | | | | | | |
| | | CAR-T | | | | | | | | |
| 4* | EC17 | 6 | 5 | nmol/kg | 5 | nmol/kg | 5 | nmol/kg | N/A | 0/0/0/3/3/0/3 |
| | T1W | million | 50 | nmol/kg | 50 | nmol/kg | 50 | nmol/kg | | |
| | (on/off) | CAR-T | 500 | nmol/kg | 500 | nmol/kg | 500 | nmol/kg | | |
| 5* | EC17 | 6 | 5 | nmol/kg | 5 | nmol/kg | 5 | nmol/kg | 5 | 0/3/3/3/0/3/3 |
| | “T1W” | million | 10 | nmol/kg | 30 | nmol/kg | 50 | nmol/kg | | |
| | M/Th/M | CAR-T | 100 | nmol/kg | 300 | nmol/kg | 500 | nmol/kg | | |

C = cycle
*Groups 4-5: skip dosing for week 2 of each cycle

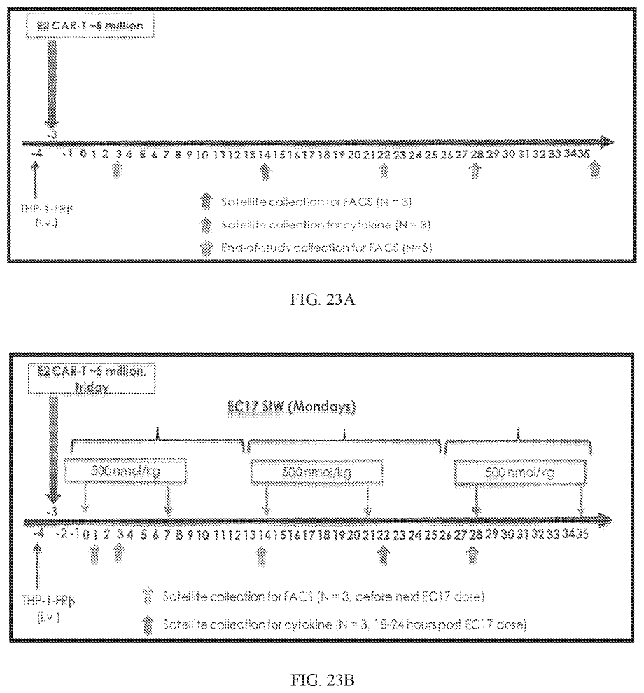

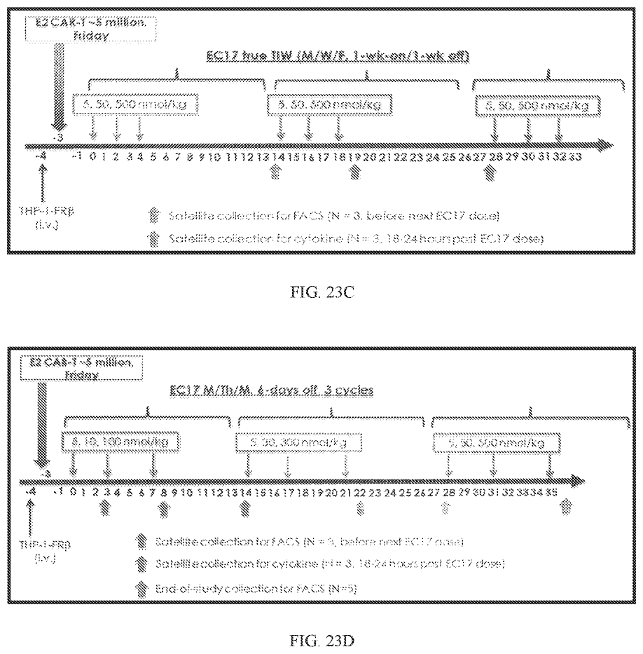

Study Schema included: Group 1 (no treatment): only for efficacy comparison. Group 2 (CAR-T only) is depicted in FIG. 23A. Group 3 (EC17 SIW) is depicted in FIG. 23B. Group 4 (EC17 true TIW) is depicted in FIG. 23C. Groups 5—EC17 low-dose “T1W” (M/Th/M, 6-day off) with/efficacy is depicted in FIG. 23D.

### A. Tumor Implantation

THP-1-FRβ AML tumor cells were grown in folate-deficient RPMI 1640 with 5-10% FBS at 37° C. in a 5% CO₂ humidified atmosphere. THP-1-FRO tumor cells in serum-free folate-deficient RPMI1640 medium were i.v. injected at 5×10⁶ cells per animal.

### B. CAR-T Cell Administration

Anti-FITC E2 scFv-CAR T cells were frozen in a T-cell freezing medium. Upon arrival, vials of frozen CAR-T cells were immediately stored at −80° C. The CAR-T cells were quickly thawed at 37° C., washed twice with CAR-T cell culture medium, and used for animal injection at 6 million viable EGFRt+E2 CAR-T cells (CD4/CD8 at ~1:1) per animal. A small aliquot was taken on the day infusion for flow cytometric analysis of E2-CAR T-cell phenotypes.

### C. Preparation of Dosing Drug Solutions

EC17 and bis-EDA-FITC dosing solutions were prepared when dosing began by weighing appropriate amounts of each compound, reconstituting in PBS, pH 7.4, sterile filtering the drug solution through a 0.22 pm PVDF syringe filter, and freezing aliquots for each day of dosing at −20° C.

### D. Compound Administration

All EC17 doses were given towards the end of day (3-4 PM) to allow potential CRS (cytokine release syndrome) to develop overnight. In the following morning, animals were scored according to a CRS grading system (see below).

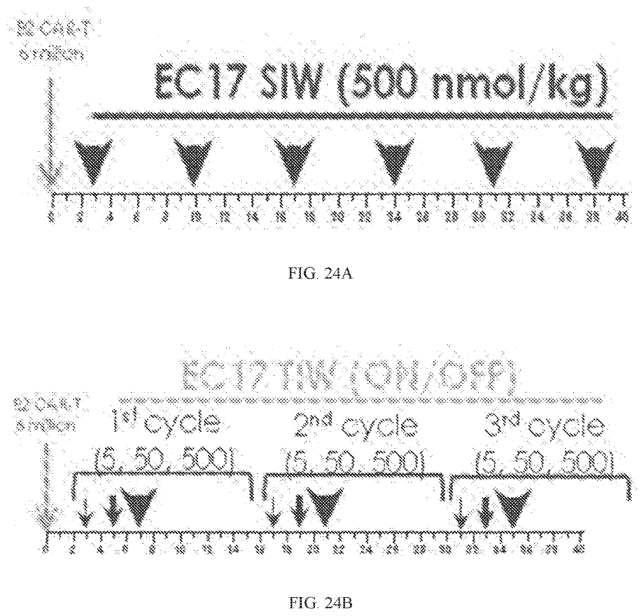

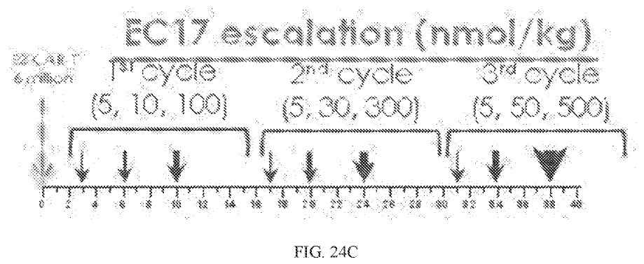

EC17 dose schedules included: Cohorts 1-2: no EC17 dose given. Cohort 3 is depicted in FIG. 24A. Cohort 4 is depicted in FIG. 24B. Cohort 5 is depicted in FIG. 24C.

### Monitoring/Efficacy:

Daily body weight measurement on the days after EC17 dose is required. Attention was given to gross animal morphology and behavior. Euthanasia was performed if mice lose a lot of weight in a short duration or when mice are approaching moribund conditions per CRS grading system or neurotoxicity. The CRS grading system shown in TABLE 2 was used.

### CRS Grading System

The CRS grading system shown in TABLE 2 was used. Tumor growth, body weight, and overall assessment: Body weight measured 2-3 times/week. On the days immediately after any EC17 dose, body weight measurement was taken daily, and attentions were given to gross animal morphology and behavior.

### Flow Cytometric Analysis

Whole blood cell analysis: Plasma was removed from predetermined volumes of whole EDTA treated blood and RBCs were lysed with RBC lysis solution. The leukocyte pellets were then resuspended in flow cytometry staining solution (1% bovine serum albumin, 50 mg/mL human IgG (Equitech Bio, cat #SLH56-0001), 0.9% sodium azide in a phosphate buffered saline, pH=7.4) and leukocyte surface marker staining was performed using the following antibodies: anti-human CD45 (clone HI30, eBioscience #47-0459-42 at 1:20 (v/v) dilution), anti-human CD137 (clone 4B4-1, BD Bioscience #564092 at 1:20 (v/v) dilution), anti-human CD8α [clone RPA-T8, BD Bioscience, catalog #557746 at 1:20 (v/v) dilution], anti-human CD4 [clone SK3, eBioscience catalog #46-0047-42 at 1:20 (v/v) dilution], anti-human EGFR [R&D systems, clone Hu1, catalog #FAB9577B @1:10(n/n)], anti-human PD1 [BD Biosciences, clone EH12.1, catalog #562511 @1:20 (v/v)], anti-human LAG3 [BD Biosciences, clone T47-530, catalog #565616 @1:20 (v/v)], anti-human TIM3 [BD Biosciences, clone 7D3, catalog #565558 @1:20 (v/v)], anti-human CD3ε [BD Biosciences, clone SK7, catalog #557832 @1:20 (v/v)]. After leukocyte staining, cells were washed with PBS and resuspended in cold PBS containing 53,000 CountBright™ beads [Invitrogen catalog #C36950] and transferred to flow cytometry collection tubes. Flow cytometry data was collected on the Gallios flow cytometer (Beckman Coulter, Brea, CA). Determination of the concentration of CAR T cells in each blood sample was calculated according to Invitrogen's instructions. CAR T cells were identified as human CD3ε+EGFRt+ events and easily distinguished and counted using the Kaluza™ flow cytometry software. The number of CAR T cells in the circulation of each infused mouse was then represented on the graphs as the total number of CAR T cells per 50 μL of whole blood analyzed. Statistical significance was determined by utilizing an unpaired, two-tailed, students t-test with significance set at p<0.05.

### Tumor and Tissue Analysis

Solid tumors (100-1000 mm³) were harvested, weighed, and minced into small pieces then transferred into 50 mL tubes containing 20 mL of a tumor digestion cocktail. The enzymatic tumor digestion cocktail consisted of 0.5 mg/mL Collagenase IV (Sigma-Aldrich, Catalog #C5138), 0.5 mg/mL Hyaluronidase (Sigma-Aldrich, Catalog #H3506) and 0.1 mg/mL DNase I (Sigma-Aldrich, Catalog #DN25) in serum-free and folate-deficient RPMI1640 medium supplemented with antibiotics. The tumor fragments were digested for one hour at 37° C. at 300 rpm on a horizontal shaker. Afterwards, the tumor digest was centrifuged at 400×g for 5 minutes and tumor cell pellet underwent a red blood cell lysis step, was then washed with cold phosphate-buffered saline (PBS, pH 7.4) and finally filtered through a 40 pm nylon cell strainer.

### Results and Conclusions

A. Three different dose schedules of EC17 administration showed different patterns of body weight loss and different levels of CRS.

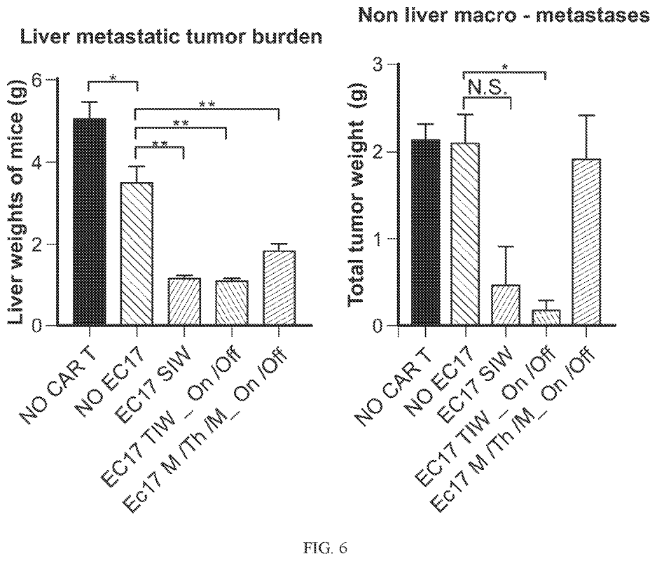

As seen in FIG. 5, Cohort 1 (no treatment) and Cohort 2 (CAR-T only) didn't show much body weight loss and had no CRS. Cohort 3 (EC17 SIW) showed EC17 dose-dependent body weight loss after each EC17 dose and showed grade 1-2 CRS throughout the study. Cohort 5 (EC17 dose escalation) showed little body weight loss during the first two cycles, but started to show similar body weight loss as cohort 2 after the end of second cycle of EC17 dose. Cohort 4 (EC17 TIW ON/OFF) showed mildest body weight loss among three treatment groups. Neither Cohort 4 or 5 showed any CRS greater than 1.

Three different dose schedules of EC17 administration showed different anti-tumor activities.

As seen in FIG. 6, liver and non-liver tumor burden were assessed on Day 39 across all cohorts. Cohort 2 (CAR-T only) didn't show any difference comparing to Cohort 1 (no treatment). Cohort 4 showed the best anti-tumor activity among the three different EC17 dose schedules.

Elimination of Circulating AMT, Cell Line. THP1-FRB

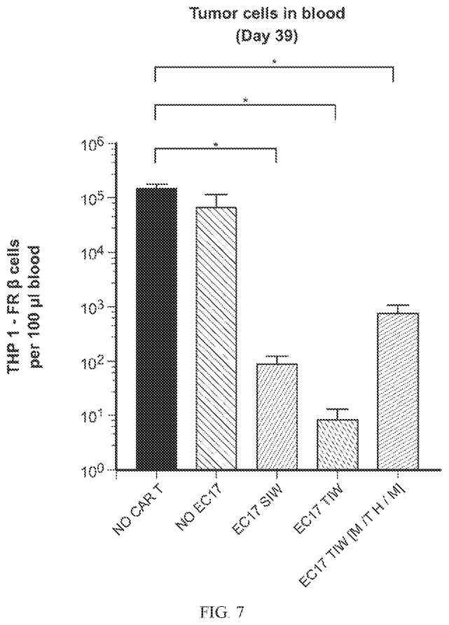

In studies using CD 19 specific anti-ALL therapy, expansion and persistence of CAR T cells in the blood has been reported to correlate with elimination of circulating CD 19+ ALL cells resulting in complete responses to therapy. In this experiment we perform a similar assay in our mouse AML model, to determine the anti-tumor activity of our adaptor controlled E2 CAR T cell by looking for the disappearance of our GFP labeled human AML cell line, THP1-FRβ, in response to different dosing regimens of the CAR T adaptor molecule, EC17. FIG. 7 displays the circulating THP1-FRP cells with the y-axis showing the total number of GFP+ cells per 100 pL of whole blood on a logarithmic scale. The different bars represent the different treatment cohorts. It was demonstrated that a high AML cell burden existed in the circulation 40 days post infusion of AML cells into mice receiving neither CAR T cells nor EC17. No reduction of AML cells in the blood was seen in mice receiving an infusion of both AML cells and E2 CAR T cells, but no EC17 treatment. This demonstrates negligible anti-tumor activity by potential allogeneic reactivity of the human T cells with the allogeneic THP1 cells. Interestingly a significant reduction in circulating THP1 cell burden in all three cohorts of mice infused with E2 CAR T cells plus EC17 at different dosing regimens was seen, thus demonstrating the effectiveness of the CAR T therapy under three different adaptor dosing regimens.

### E2-CAR-T Exhaustion Phenotypes

Under chronic antigen stimulation, for example in instances of chronic viral infections or cancer, T cells undergo a process of exhaustion where they are no longer able to proliferate, secrete inflammatory cytokines or kill antigen presenting target cells. CAR T cells also possess the potential for exhaustion under chronic stimulation of the CAR by constant presence of scFv stimulating antigen. Because our E2 CAR T cells only react to the presence of our bridge molecule, EC17, bound to the surface of FR+ tumor cells, we have the ability to prevent chronic antigen stimulation of the E2 CAR T cells by ceasing treatment with EC17 and hence removing the presence of surface bound antigen. The rest period characterized by the absence of surface antigen prevents chronic antigen exposure of E2 CAR T cells and prevents the resulting exhaustion that could result.

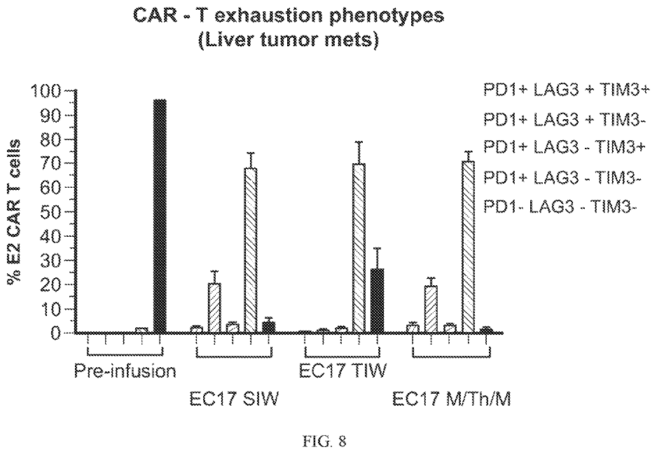

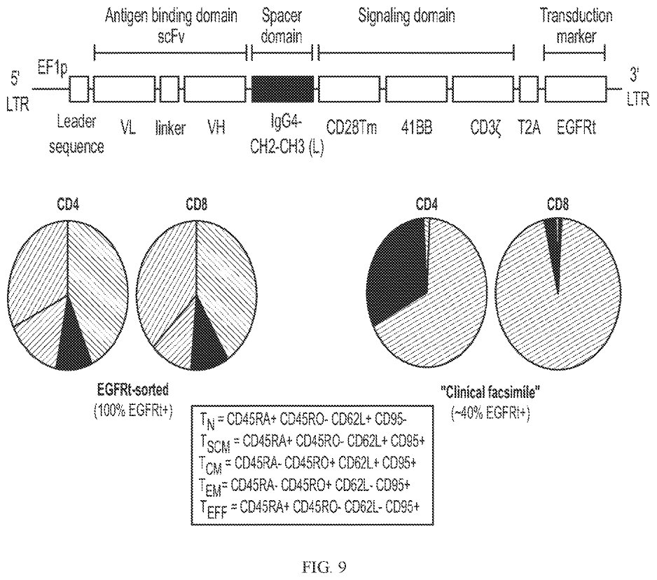

To confirm that the EC17 dosing regimens did not result in E2 CAR T cell exhaustion, flow cytometry analysis was performed on single cell preparations from THP1-FR liver metastases and surface markers which are expressed specifically on exhausted T cells. As T cells approach exhaustion, there is an increase in the co-expression of surface inhibitory receptors, PD1, LAG3 and TIM3. FIG. 8 shows a bar graph where the y-axis illustrates the percentage of the total E2 CAR T cells isolated from the solid liver tumors, which express the various combinations of the three surface markers which is represented by the different bars. Of note, fully exhausted T cells which simultaneously express all three markers are represented by the left-hand bar in each group. The left group shows the pre-infusion of E2 CAR T cells to the liver tumor CAR T cells isolated from the three different EC17 treated cohorts. The inhibitory receptor expression is almost zero in the pre-infusion CAR T cell product as most of the T cells are negative for all three surface markers. Importantly, all three EC17 treated cohort E2 CAR T cells possessed the exhausted triple positive cells and interestingly, cohort 4 (EC17 TIW) expresses the fewest number of double and triple positive events and further consisted of a significant number of triple negative CAR T cells. These data suggest that all three EC17 dosing regimens utilized in this mouse experiment might be useful in the clinic in that there is significant anti-tumor activity and there is very little induction of CAR T exhaustion.

### Example 16—Cell Lines and Reagents

Unless otherwise noted, all FR+ and FR-negative cancer cell lines were maintained in RPMI-1640 medium (Gibco BRL) supplemented with 10% heat-inactivated fetal calf serum without (FFRPMI) or with (RPMI) 2.4 mM folic acid (FA). KB (FRα-expressing human cervical carcinoma with HeLa markers) and CHO-b (Chinese hamster ovary cells transfected with human FRβ) were used as the sources of FRα and FRβ) for radioligand binding assays, respectively. MDA-MB-231 represents a FRα subclone of human TNBC (triple negative breast cancer) cell line. For AML studies, a green fluorescent protein (GFP)-expressing isogenic pairs of FRβ-positive (THP1-FRβ) and FR-negative (THP1-FG12) cell lines were provided. Both were established from THP-1 (ATCC, TIB-202), a commonly used cell model for researching pediatric AML which was originally derived from a 1-year-old male infant with acute monocytic leukemia. For osteosarcoma studies, HOS-FRα was established by lentiviral transduction of FR-negative HOS-143b (ATCC, CRL8303) with FOLR1 gene encoding the human FRα. HOS-143b is originally established from a primary tumor of a 13-year-old Caucasian female and is highly tumorigenic in NSG mice. The GFP-expressing bioluminescent pairs of FR+ HOS-FRαfLuc and FR-negative HOS-143bfLuc were transduced with lentiviral firefly luciferase. LEGENDplex™ human cytokine panels were purchased from BioLegend (San Diego, CA). The lactate dehydrogenase (LDH) based CytoTox 96® non-radioactive cytotoxicity assay kit was purchased from Promega (Madison, WI). Commercially available anti-human antibodies used for multicolor flow cytometry were: CD45RA (clone HI100), CD45RO (clone UCHL1), CD4 (clone SK3), and CD69 (clone FN50) from Thermo Fisher Scientific (Waltham, MA); CD3ε (clone SK7), CD8α (clone RPA-T8), CD137/4-1BB (clone 4B4-1), CD25 (clone M-A251), PD1 (clone EH12.1), LAG3 (clone T47-530), and TIM3 (clone 7D3) from BD Bioscience (San Jose, CA); biotinylated anti-human EGFR (Cetuximab, clone Hu1) from R&D systems (Minneapolis, MN); and FRα (clone LK26) from BioLegend (San Diego, CA). A fluorophore-conjugated anti-biotin was also purchased from BioLegend. APC-conjugated anti-FITC mouse IgG2a/kappa antibody (clone NAWESLEE), CountBright™ beads (Invitrogen), Annexin V staining buffer, and AlexaFluor- 647-conjugated Annexin V were purchased from Thermo Fisher Scientific. For enzymatic digestion of tumor tissues, collagenase 1V, hyaluronidase and DNase I were all purchased from Sigma-Aldrich (St. Louis, MO).

EC17 or folate-FITC [FA-(γ)-ethylenediamine-FITC] was synthesized at Endocyte. 3H-EC17 was either purchased from Moravek biochemicals (Brea, CA) at a specific activity of ~0.952 Ci/mmol or prepared at Endocyte by conjugating FITC with 3 H-F A-(γ)-ethylenediamine made by ViTrax (Placentia, CA) at a specific activity of ~1.2 Ci/mmol.3H-FA was also purchased from ViTrax at a specific activity of 59 Ci/mmol. For CRS rescue, sodium fluorescein dosing solution was diluted from AK-FLUOR® 25% (fluorescein injection, USP) which was purchased from Purdue Pharmacy (NDC 17478-250-25).

### Example 17—Humanized CAR Construct and CAR-Modified T Cells

Previous studies used a GFP+ second-generation anti-FITC scFv (clone 4M5.3) CAR containing the hinge and transmembrane sequences of CD8α and 4-1 BB/CD3ζ signaling domains (i.e., FITC-4M5.3-scFfv-CD8αhinge-CD8αtm-4-1BB/CD3ζ). For translation into first-in-human testing, the second-generation fully human FITC-specific (clone E2) CAR construct (herein referred to as E2) was developed (FIG. 9, top diagram). CAR-modified T cells are shown in FIG. 9, bottom pie charts.

The construct described herein is a FITC-specific CAR construct including (1) a fully human anti-FITC scFv (clone E2, Kd=0.75 nM), (2) an IgG4 hinge-CH2(F235D, N297Q)-CH3 spacer fused to a CD28-transmembrane domain, (3) a second-generation 4-1 BB/CD3-endodomain, and (4) a cell-surface human EGFRt tag (FIG. 9, top diagram) (SEQ ID NO:04 and SEQ ID NO:05 are the nucleic acid and amino acid sequences, respectively). To generate CAR-modified T cells, lend virus was produced in 293T cells co-transfected with CAR-encoding epHIV7 lentiviral vector. Donor CD4+ and CD8+ T cells were purified by immunomagnetic selection and transduced separately or at about a 50:50 ratio. In general, only one round of CD3/CD28 bead activation followed by one or two rounds of rapid in vitro expansion were carried out. For preclinical evaluations, several batches of EGFRt-sorted CD4, CD8 and unsorted CD4/CD8 CAR-T cells were used. All CAR-T cell preparations were analyzed prior to cryopreservation and after thawing to determine EGFRt expression and CD4/CD8 ratios by flow cytometry. Using combinations of surface markers, differentiation status of CD4+ and CD8+ CAR-T cell subsets on the day of infusion was analyzed and defined as TN, CD45RA+ CD45RO− CD62F+ CD95− naive T cells; TSCM. CD45RA+CD45RO− CD62F+CD95+ stem cell memory T cells, TCM, CD45RA− CD45RO+ CD62F+ CD95+ central memory T cells; TEM, CD45RA−CD45RO+CD62L− CD95+ effector memory cells; and TEFF, CD45RA+ CD45RO− CD62L−CD95+ effector T cells. For preclinical testing described below, studies included two batches of EGFRt-sorted pure CD4 and CD8 subsets (after mixing at about 1:1 ratios) and several batches of unsorted ~1:1 EGFRt+ CD4/CD8 admixture including a “clinical facsimile” preparation with low differentiation profiles.

Amid a series of different CAR constructs synthesized and evaluated, the fully human anti-FITC scFv (FITC-E2) CAR was chosen for preclinical development (FIG. 9). This second-generation fully human CAR consisted of anti-FITC scFv (clone E2), an IgG4-Fc spacer/hinge with double mutations in the CH2 region (L235D and N297Q) to reduce binding to FcyR, a CD28 transmembrane domain, and 4-1BB/CD3 signaling domains appended to a cell-surface EGFRt tag by a T2A ribosomal skip sequence (i.e., FITC-E2-scFv-IgG4hinge-CD28tm-4-1BB/CD3ζ-T2A-EGFRt). For preclinical studies, both EGFRt-sorted and unsorted E2 CAR-T cells were prepared at ~1:1 CD4/CD8 ratios, and T cell subtype phenotyping was routinely performed by flow cytometry at the time of CAR T cell infusion (day 0) for each in vivo experiment. A typical expression pattern of EGFRt-sorted CAR-T cells included both CD4 and CD8 subsets at approximately 42% TSCM, 10% TCM, 12% TEM and 34% TEFF (FIG. 9, pie charts on the left). Only EGFRt-sorted CAR-T cells were used for co-culture and pharmacokinetic studies. For tumor therapy, a “clinical facsimile” batch with a low differentiation profile (FIG. 9, pie charts on the right) for MDA-MB-231 was used, and a research batch was used for THP1-FRβ) and HOS-FRα studies. The “clinical facsimile” batch (~39% EGFRt+) included CD4+ subsets at −66% TSCM and −32% TCM and CD8 subsets at ~95% TSCM and about 3% TCM. The research batch (~23% EGFRt+) was more differentiated and included CD4 subsets at about 32% TSCM, about 53% TCM, about 11% TEM and about 3.7% TEFF and CD8 subsets at about 44% TSCM, about 0.28% TCM, about 3.4% TEM and about 52% TEFF.

### Example 18—EC17 CAM's Bispecific Affinity

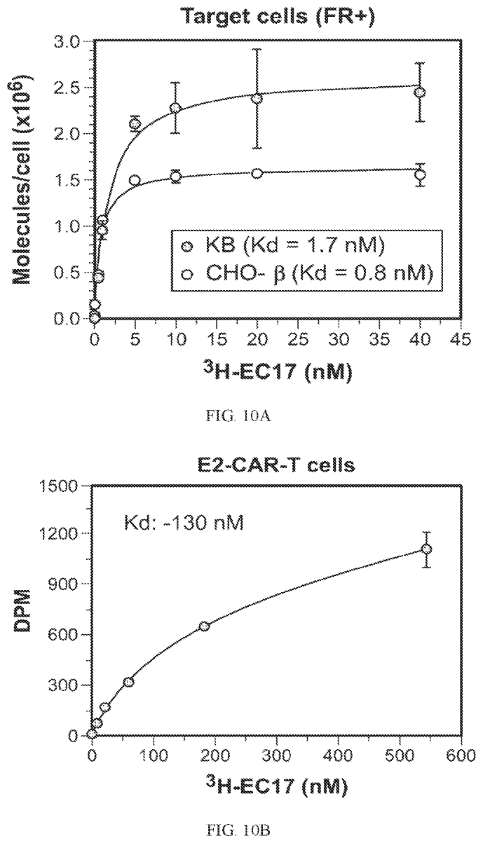

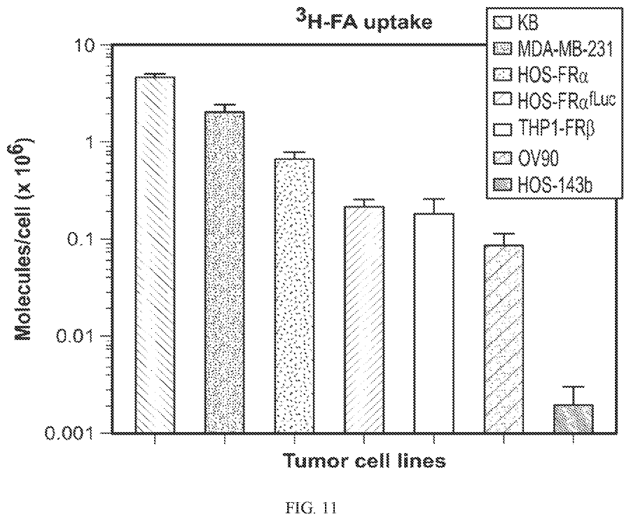

The bispecific affinities of EC17 CAM (a CAM is equivalent to a “bridge” or the “compound” in this application) were assessed using 3H-EC17 in cell-based radioligand binding assays. For binding to FR+ targets, KB and CHO-b cells were pre-seeded overnight in 24-well tissue culture plates and incubated with 0.1, 0.5, 1, 5, 10, 20, and 40 nM of 3H-EC17 in FFRPMI for 2 h at 37° C. Afterwards, the cells were rinsed with phosphate-buffered saline (PBS, pH 7.4) and lysed with 1% sodium dodecylsulfate. The whole cell lysates were quantitated for the level of radioactivity and cellular protein content by standard Pierce BCA protein assay. The number of 3H-EC17 molecules bound per cell was calculated to determine the dissociation constants (Kd) for FRα (KB) and FRβ (CHO-β) respectively (FIG. 10). The EC17 has already been tested in the clinic for immunotherapy and optical imaging purposes. To directly quantify its bispecific binding affinities, ³H-EC17 was synthesized and radioligand binding assays were carried out on KB and CHO-β cell lines representing FRα+ and FRβ+ target cells, respectively, and on unsorted EGFRt CAR-T cells representing the effector cells. When binding to its targets, EC17 demonstrated similar affinities towards both FRα and FRβ with low Kd values of 1.7 nM and 0.8 nM, respectively (FIG. 10, Panel A).). Upon binding to unsorted E2-CAR-T cells (−24% EGFRt+, −95:5 CD8/CD4 ratio), the Kd value was estimated at −130 nM (FIG. 10, Panel B).

### Example 19—Tumor Models

All animal care and use were performed according to NIH guidelines and in compliance with protocols approved by the Purdue University Animal Use and Care Committee. Female 4 to 5-week-old NOD/SCID gamma (NSG™) mice (stock number:005557) were purchased from The Jackson Faboratory (Bar Harbor, ME). Unless specifically indicated, all animals were maintained on a FA-deficient diet (TestDiet, St. Fouis, MO) upon arrival and throughout the study. To establish subcutaneous xenografts, MDA-MB-231 and HOS-FRα were implanted in the right flank region at 2.5×10⁶ and 1×10⁶ cells per animal, respectively. For intravenous xenografts, THP1-FRβ) cells were inoculated at 5×10⁶ cells per animal. Subcutaneous tumors were measured 2-3 times per week with a caliper and calculated using the ellipsoidal formula (length×width2)/2. Euthanasia was performed per study design or when (i) the animals had lost ≥20% of body weight or approached moribund conditions, (ii) subcutaneous tumors reached ≥1500 mm³ in size, or (iii) animals displayed signs of swollen belly and severe distress (i.e., THP1-FRβ). All animal doses (CAR-T cells, EC17, sodium fluorescein) were given intravenously.

### Example 20—Tumor Therapies

In a therapeutic setting, EC17 CAM can be given before or after CAR-T cell injection. As described herein, the first dose of EC17 was administered 2-3.5 days after CAR-T cells to allow for an observation period of human T cells in tumor-bearing mice. Two batches of unsorted E2-CAR-T cells (23% or 39% EGFRt+, 1:1 CD4/CD8) were used for in vivo studies. On the day of infusion for each experiment (day 0), frozen CAR-T cells were quickly thawed at 37° C., washed 2× with Dulbecco's IX PBS (pH 7.4) and injected into the tail vein at desired EGFRt+E2-CAR-T cell doses. In addition, a small aliquot of CAR-T cells was analyzed by flow cytometry for CD4 to CD 8 ratio and differentiation status of CAR-T cells. On the first day of EC17 dose, tumor-bearing animals were randomly assigned into groups according to their tumor sizes or the same number of days post intravenous implantation (i.e., THP1-FRβ).

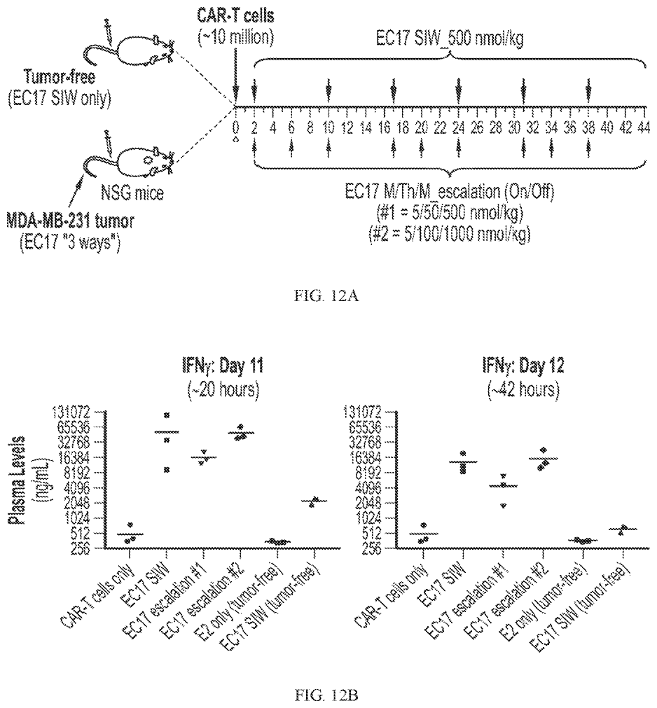

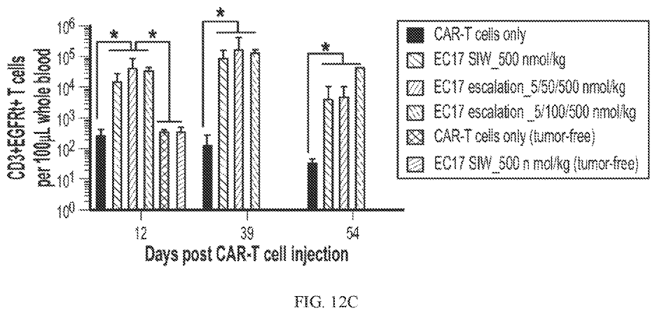

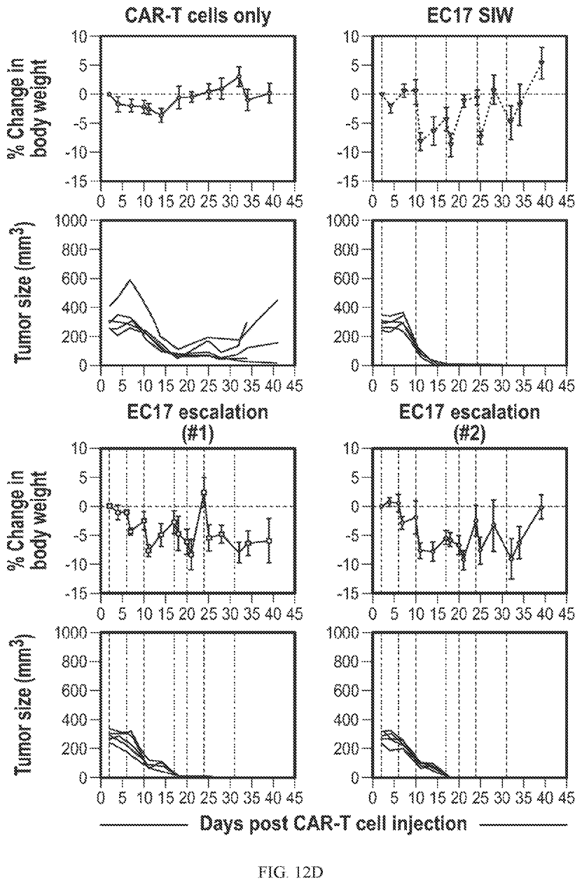

For MDA-MB-231 studies, mice received a high dose (~10 million) of a “clinical facsimile” of E2-CAR-T cells (~39% EGFRt+) with a low differentiation profile (FIG. 9). Two days later, 3 different treatment regimens of EC17 were started at an average tumor size of −293±39 mm³ (FIG. 12). The EC17 dosing was given once-a-week (SIW) at 500 nmol/kg on Mondays, or as escalating doses of 5, 50 or 100, and 500 or 1000 nmol/kg (i.e., 5/50/500 or 5/100/1000) on Monday, Thursday, and Monday with a 6-day break in-between cycles. Control mice were left untreated (received CAR-T cells but no EC17). For comparison, a cohort of tumor-free littermates also received the same number of CAR-T cells without or with EC17 SIW at 500 nmol/kg.

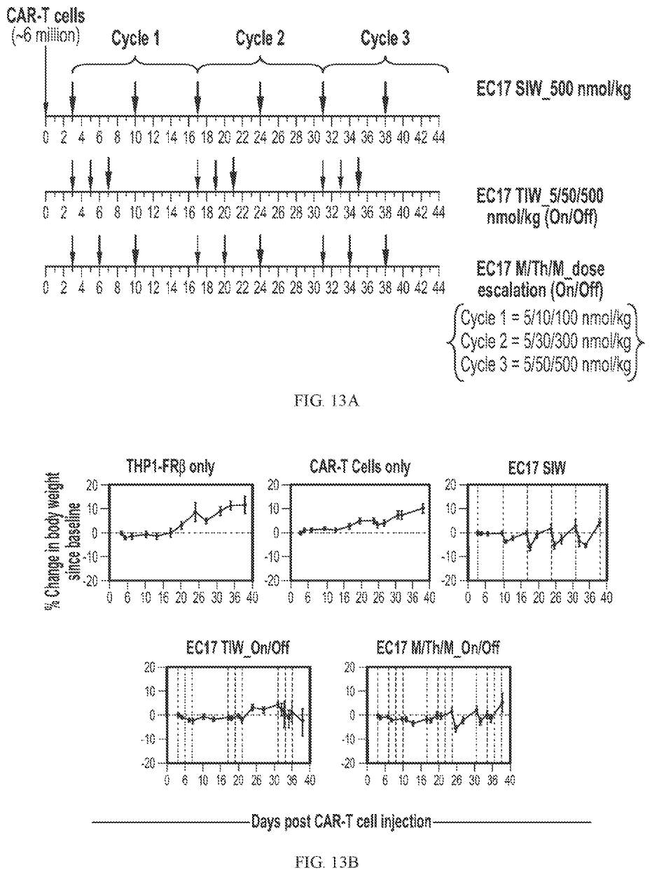

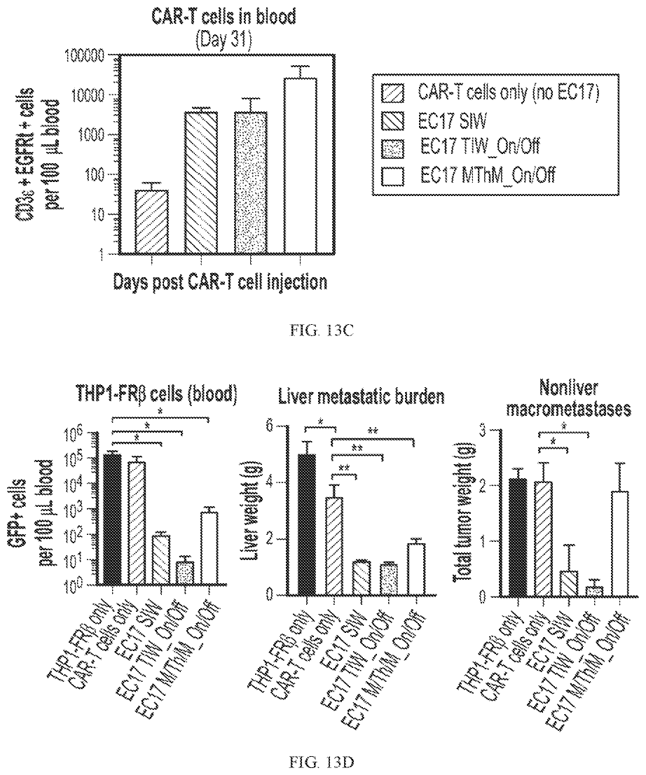

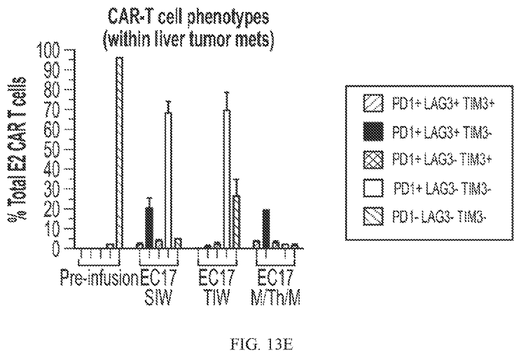

For AML studies (FIG. 13), mice were intravenously infused with THP1-FRβ tumor cells one day prior to receiving a low dose of −6 million E2-CAR-T cells (−23% EGFRt+). At ~3.5 days post CAR-T cell infusion, EC17 was dosed in 3 different ways including i) SIW at 500 nmol/kg, ii) thrice at 5/50/500 nmol/kg on Monday/Wednesday/Friday followed by a 9-day break in between cycles (T1W On/Off), and iii) as escalating doses of 5/10/100 nmol/kg in Cycle 1, 5/30/300 nmol/kg in Cycle 2, and 5/50/500 nmol/kg in Cycle 3, all on Monday/Thursday/Monday with a 6-day break in between cycles (M/Th/M_On/Off). On day 31, satellite animals were harvested for quantification of CAR-positive T cells identified in the mouse blood as human CD3ε+EGFRt+ events and calculated as absolute numbers per 100 μL of whole blood. Upon euthanasia or at the end of study on day 38, total tumor load in THP1-FRβ tumor-bearing mice was assessed by measuring GFP+ tumor cells in the blood by flow cytometry and collecting liver weight (with metastatic lesions) and total weight of nonliver macrometastases found in the body. Tumor fragments of liver metastases were enzymatically digested into single-cell suspensions and viable cell populations were analyzed for the status of CAR-T cell exhaustion using anti-human PD1, LAG3 and TIM3 (clones EH12.1, T47-530 and 7D3, respectively).

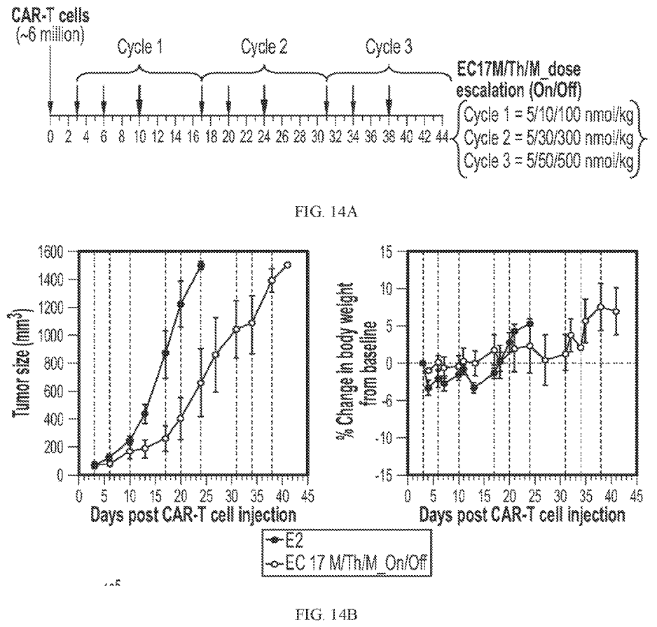

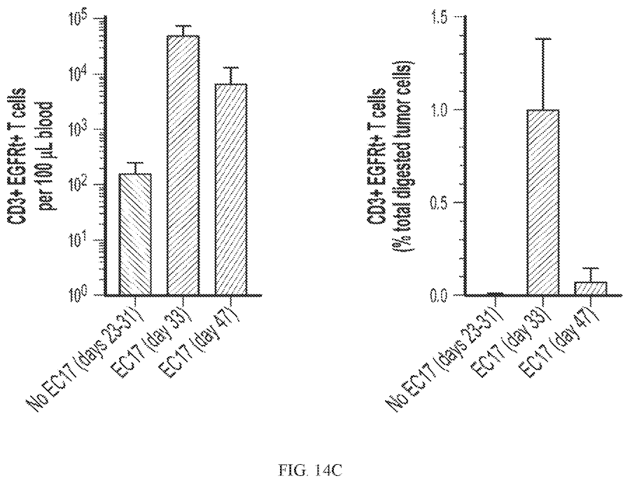

For the osteosarcoma study (FIG. 14), two cohorts of mice were subcutaneously implanted with HOS-FRα tumor cells 3 days prior to receiving ~6 million of the same CAR-T cell preparation used in the THP1-FRβ) study (FIG. 13). At ~3.5 days post CAR-T cell infusion, one cohort of mice was given up to 3 cycles of EC17 at 5/10/100 nmol/kg in Cycle 1, 5/30/300 nmol/kg in Cycle 2, and 5/50/500 nmol/kg in Cycle 3, all following the Monday/Thursday/Monday regimen with a 6-day break in-between cycles. At the end of study, circulating CD3ε+EGFRt+ CAR-T cells per 100 μL of mouse blood were enumerated. HOS-FRα tumors (+/−EC17 treatment) were also harvested and digested for flow cytometric analysis of tumor-infiltrating CAR-T cells.

### A Dose Escalation Trial Against an Aggressive Osteosarcoma Model

For our intended purpose, HOS-FRα is a low FR-expressing but most aggressive tumor model with a functional FR level of ~5.82±1.45 pmol/mg protein. In parallel to the THP1-FRβ) study described above, two cohorts of mice with 3-day-old HOS-FRα tumors were given the same E2-CAR-T cells at the same dose (~6 million). One cohort was treated with the same accelerated EC17 dose escalation regimen at 5/10/100 nmol/kg in Cycle 1, 5/30/300 nmol/kg in Cycle 2, and 5/50/500 nmol/kg in Cycle 3, on Monday/Thursday/Monday schedule followed by a 6-day break (FIG. 14, panel A). As HOS-FRα tumors grew aggressively without EC17 treatment, accelerating EC17 dose escalation at this CAR-T cell dose was safe (no CRS or body weight loss) and resulted in a significant delay in tumor growth within the first two cycles of treatment (FIG. 14, panel B). Upon protocol-mandated euthanasia due only to tumor size (≥1500 mm3), flow cytometric analyses performed on whole blood showed an EC17-dependent CAR-T cell expansion up to day 47 but higher on day 33 (FIG. 14, panel C, left bar graph).

Ex vivo tumor analysis on day 33 indicated a low but significant intratumoral CD3e+EGFRt+ CAR-T cell population in EC17-treated animals which amounted to −1% total viable digested tumor-derived cells (FIG. 14, panel C, right bar graph). As large HOS-FRα tumors stopped responding to treatment in Cycle 3 (FIG. 14, panel B), intratumoral CAR-T cells also diminished on day 47(FIG. 14, panel C) Notably, HOS-FRα tumors analyzed by a 3H-FA radioligand assay upon disease progression showed similar FRα levels with and without EC17 treatment. Thus, it appeared that HOS-FRα tumors in NSG mice quickly outgrew the tumor infiltrating capability of CAR-T cells and may have decreased cytolytic activity due to T cell exhaustion as suggested by in vitro co-culture studies.

### EC17 Dose Finding Study in Tumor-Containing and Tumor-Free Mice

The initial EC17 dose finding studies were conducted in MDA-MB-231 tumor bearing mice as shown by the schematic diagram of the experimental layout (FIG. 12, panel A). NSG mice without or with MDA-MB-231 tumors (~211±65 mm3) were engrafted on day 0 with −10 million of a “clinical facsimile” batch of E2-CAR-T cells (−39% EGFRt+, 51:49 CD4/CD8). This batch of CAR-T cells consisted of mostly TSCM and TCM phenotypes (see FIG. 9). Two days after the CAR-T cell injection, one cohort of tumor-bearing mice were left untreated while three cohorts were treated with different regimens of EC17, including single injection per week (SIW) at 500 nmol/kg, or as escalating EC17 dose levels of 5/50/500 (Escalation-1) or 5/100/1000 nmol/kg (Escalation-2) given on a Monday/Thursday/Monday schedule with 1-week drug-free intervals. For comparison, two tumor-free cohorts were either untreated or treated with EC17 SIW at 500 nmol/kg.

Human T cell-derived IFNγ levels in mouse blood were measured in all cohorts using satellite animals on days 11 and 12 (−20 and 42 hours after the previous EC17 dose). Compared to tumor-bearing mice that received CAR-T cells only, all EC17 treated tumor cohorts had ~30× (day 11) and ~10× (day 12) higher IENγ production in mouse plasma, and the levels of this cytokine decreased naturally from 20 hours to 42 hours later (FIG. 12, panel B). In accordance with IFNγ release, EC17 also triggered CAR-T cell expansion identified as human CD3e+EGFRt+ events in mouse blood, and cells persisted up to 54 days in tumor-bearing animals (last measurement) (FIG. 12, panel C). In tumor-free cohorts, no CAR-T cell expansion was detected by flow cytometry, but low levels of IFNγ were detected on days 11 and 12 in animals that received the same number of CAR-T cells plus EC17 (FIG. 12, panels B-C). Moreover, no CRS symptoms (grade 0 out of a 0-5 scale) or body weight loss was observed in tumor-free mice that received two weekly doses of EC17 at 500 nmol/kg.

Moderate-to-severe CRS symptoms (grades 2-3) and significant body weight losses (FIG. 12, panel D) were observed in tumor-bearing cohorts with continued EC17 dosing independent of the regimen. While EC17 SIW at 500 nmol/kg caused the earliest onset of CRS and body weight loss, the aggressive EC17 Escalation-2 regimen (up to 1000 nmol/kg) caused persistent body weight loss with animal recovery occurring after EC17 dose cessation (FIG. 12, panel D, top row). Notably, symptoms of graft-versus-host disease (GVHD) included red itching skin and hairlessness and became obvious at −1 month after CAR-T cell engraftment. Although animals receiving only CAR-T cells showed signs of nonspecific CAR-T cell/tumor alloreactivity, only EC17-treated cohorts produced 100% cures (FIG. 12, panel D, bottom row). Therefore, EC17 administration in the presence of FR+ tumors was the key to drive i) CAR-T cell activation, ii) cytokine production, and iii) in vivo CAR-T cell expansion and persistence. Under specific conditions, however, severe CRS (grade≥3) was triggered by a high CAR-T cell dose in combination with EC17 doses equal to or greater than 500 nmol/kg.

### EC17 CAM Dosing Control in Anti-Leukemic Activity

Intravenously implanted GFP-expressing THP1-FRβ) tumor cells developed disseminated diseases in NSG mice with tumor cells in the circulation and liver/non-liver metastases throughout the body. THP1-FRβ) tumor cells could also localize to the mouse ovary which appeared inflamed during the early stage of tumor progression. Therefore, total tumor burden in each animal in study cohorts was assessed by quantitating circulating GFP+ tumor cells in the blood, liver weights, and all-inclusive non-liver macrometastases visible to the naked eye. Although THP1-FRβ) expressed a low level of FR in vitro, THP1-FRβ) tumor metastases were found to express a higher than expected functional FRs level at −8.9±2.8 pmol/mg membrane protein. Thus, THP1-FRβ) tumor-bearing mice were engrafted with a research batch of EGFRt-unsorted E2-CAR-T cells (−23% EGFRt+, 1:1 CD4:CD8) at −6 million/animal and then treated with 3 different EC17 dosing regimens (FIG. 13). Starting 3 days after CAR-T cell injection, EC17 dosing regimens began as SIW at 500 nmol/kg continuously, three times a week (TIW) at 5/50/500 nmol/kg on Monday/Wednesday/Friday followed by a 9-day break, or by an accelerated dose escalation regimen at 5/10/100 nmol/kg in Cycle 1, 5/30/300 nmol/kg in Cycle 2, and 5/50/500 nmol/kg in Cycle 3, on Monday/Thursday/Monday (M/Th/M) followed by a 6-day break (FIG. 13, panel A). While some body weight loss and grade 1-2 CRS in Cycles 2 and 3 were observed in animals receiving EC17 SIW treatment, animals that received EC17 TIW had the least body weight loss with grade 0-1 CRS only (FIG. 13, panel B). Amongst the animals that received the 3 cycles of EC17 M/Th/M dose escalation, grade 0-1 CRS and very mild body weight loss were observed in Cycle 2 after the last dose of EC17 of 300 nmol/kg. Using satellite animals on day 31, CAR-T cells were enumerated in the blood and the data demonstrated EC17-dependent CAR-T cell expansion and persistence in all treated cohorts (FIG. 13, panel C). Compared to control animals that received tumor cells only or tumor cells plus CAR-T cells without EC17, EC17 dosed with any of the 3 “intra-patient” escalation formats effectively reduced circulating THP11-FRβ tumor cells in the blood and showed similar activities against liver tumor metastases (FIG. 13, panel D, left and middle bar graphs). Only minor allogeneic reactivity was seen against THP1-FRβ) liver metastases in mice that received CAR-T cells only. While EC17 SIW and TIW at 10-fold dose escalation (on/off) successfully controlled non-liver macrometastases, EC17 M/Th/M dose escalation at a slow pace per cycle (FIG. 13, panel A) failed to control the non-liver macrometastases (FIG. 13, panel D, far right panel). At the end of study (i.e., 39 days post CAR-T cell injection), CAR-T cells isolated from liver THP1-FRβ) tumor metastases appeared to have the least expression of double- and triple-positive T cell inhibitory receptors, PD1, LAG3 and TIM3 (FIG. 13, panel E). Overall, no severe CRS (i.e., grades≥3) was observed in any EC17 treated cohorts. Nevertheless, the more aggressive EC17 TIW dose escalation (on/off) schedule trended to be the best regimen for reducing overall THP1-FR) tumor burden in these mice.

### Example 21—Functional FR Assessments

These functional FR assessments apply to examples described herein. Besides pediatric cancer cell lines transfected with FRα (HOS-FRα) and FRβ) (THP1-FRβ), cancer cell lines of different histology and FR expression levels (FIG. 11) were included. As estimated by a radioligand binding assay (100 nM ³H-FA, 1 h at 37° C.), the ranking order of total available FRs on these cell lines was: 9×10⁴ (OV90, a low-FR expressing ovarian cancer cell line), 1.9×10⁵ (THP1-FRβ), 2.4×10⁵ (HOS-FRαfLuc), 7×10⁵ (HOS-FRα), 2.1×10⁶ (MDA-MB-231) and 4.8×10⁶ (KB) FA molecules/cell. Also included as FR-negative controls were HOS-143b^{(fLuc)} and THP1-FG12 parent cell lines. Thus, the general ranking of functional FR expression on co-cultured FR+ cancer cell lines was: KB>MDA-MB-231>HOS-FRα>HOS-FR^{αfLuc}>THP1-FRβ (AML)>OV90.

### Example 22—Statistics

Statistical analyses were performed using the computer program GraphPad Prism (GraphPad Software Inc., San Diego, CA). Data were analyzed using Student's t-test or one-way ANOVA. If applicable, data were further analyzed across treatment groups using appropriate multiple comparison post-test. *=p<0.05 was considered statistically significant in all tests.

### Example 23—Study of Fluorescein-Specific CAR T Cells in Combination with Parenterally Administered Folate-Fluorescein for Osteo-Genic Sarcoma

This Phase 1, open-label, non-randomized study will enroll research participants with recurrent or refractory osteosarcoma to examine the safety and feasibility of administering autologous, peripheral blood-derived CD4 and CD8 T cells that have been genetically modified using a third generation self-inactivating (SIN) lentiviral vector to express a fluorescein (FL)-specific human scFv second generation (4-1BB:zeta) chimeric antigen receptor (CAR) and EGFRt in combination with UB-TT170 (previously referred to as EC17; BB-IND 010704, Sponsor: Umoja Biopharma), a conjugate of folate and fluorescein (FL). AntiFL(FITC-E2) CAR T cells are posited to not be reactive to human cells but can recognize and target folate receptor positive target cells in the presence of a small molecule adaptor drug consisting of folate conjugated to FL. Thus, control of “ON” stage reactivity of antiFL(FITC-E2) CAR T cells is dependent on dosing of the small molecule drug and can be rapidly reversed by dosing of free fluorescein. This system therefore affords a new level of control of CAR T cells in near real time based on physician dictated dosing of UB-TT170.

Response by fixed dosing of antiFL(FITC-E2) CAR T cells followed by recursive escalating doses of UB-TT170 is expected to be a function of individual subject's tumor burden, T cell proliferation and cytokine production, and the degree to which the individual subject's tumor/tumor associated myeloid cells and macrophages express the receptors (FR) alpha and/or beta. Therefore, this study is designed to treat subjects with intra-subject dose escalation of UB-TT170 to identify their individual UB-TT170 optimized dose, defined as a dose that elicits an anti-tumor response in a controlled and safe manner.

The primary objective of this study is to identify a recommended dose escalation sequence of UB-TT170 following administration of antiFL(FITC-E2) CAR T cells for further clinical development. This safety objective for the study includes an assessment of the safety and tolerability of cellular immunotherapy utilizing ex-vivo expanded autologous T cells genetically modified to express the antiFL(FITC-E2) CAR when administered prior to dosing of the UB-TT170. The study is a 3+3 design exploring the tolerability of 3 different dose increase sequences of UB-TT170.

Secondary objectives include assessing the feasibility of manufacturing antiFL(FITC-E2) CAR T cells from subject-derived apheresis products using a 7-day single train (CD4/8 combined) T cell manufacturing platform, studying the in vivo engraftment and persistence of transferred cells by flow cytometry and/or quantitative PCR for lentiviral vector-specific sequence, evaluating pharmacokinetics of UB-TT170 and quantifying anti-tumor responses by measuring changes in tumor burden using disease-specific evaluations. Exploratory objectives include evaluating expression of folate receptor alpha or beta in archival tumor or tissue specimens or obtained from subjects during conduct of the trial, evaluating the presence of adoptively transferred T cells in tumor tissue and/or normal tissue derived from subjects, and analyze blood, bone marrow, CSF, normal tissue, and/or tumor tissue for biomarkers of safety and/or anti-tumor activity.

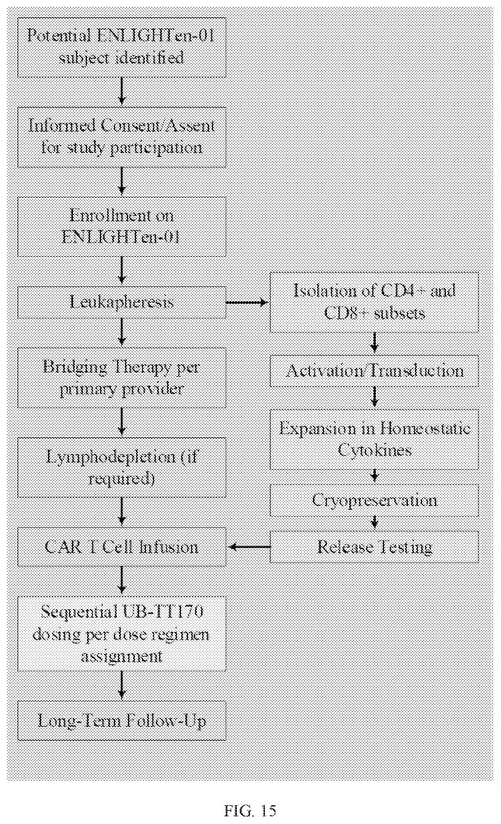

T cells will be isolated from an apheresis product obtained from the research participant after study enrollment. After infusion of antiFL(FITC-E2) CAR cells on Cycle 1, Day 0 (C1D0), subjects will receive increasing doses of UB-TT170 on C1D4, C1D7 and C1D11 in order to identify the maximum tolerated dose of UB-TT170 for the subject. This subject-specific maximum tolerated dose will then be administered weekly for a total of 2 additional weeks at which time restaging will occur. Subjects with stable disease or disease responsive to treatment may continue on study for up to 3 subsequent courses, each consisting of 7 weekly doses for a total of 4 complete courses. An experimental design scheme is depicted in FIG. 15.

The Phase 1 study design will provide pilot data on the safety and efficacy of administering anti-FL E2-CAR T cells followed by recurrent dosing of UB-TT170. Response to combination treatment with antiFL(FITC-E2) specific chimeric antigen receptor (CAR) in combination with UB-TT170 is expected to be a function of individual subject's tumor burden, T cell proliferation and cytokine production, and the degree to which the individual subject's tumor expresses the folate receptors alpha or beta. In contrast to many “standard” oncology MTD/dose finding studies, this study is designed to treat research participants with increasing doses of UB-TT170 in order to identify their individual UB-TT170 optimized dose.

### Example 24—Single-Agent UB-TT170 (Previously Known as EC17), Human Experience and Dose Justification

The safety of UB-TT170 administered as a single agent in humans has been assessed in multiple exploratory pilot and Phase 2 studies. Subjects received 0.1-0.3 mg/kg UB-TT170 prior to intraoperative imaging of tumors. The most common adverse event reported in 10.7% of the subjects (9/84) was a mild hypersensitivity reaction, with symptoms of hives, abdominal discomfort, itching throat and sneezing. Other adverse events following intravenous UB-TT170 administration were vomiting, abdominal discomfort, and injection site reaction. No serious adverse events were reported. Overall, injection of UB-TT170 alone for intraoperative imaging of tumors was well tolerated.

### Dosing Rationale

This study will examine the safety and feasibility of administering autologous, peripheral blood-derived T cells that have been genetically modified using a SIN lentiviral vector to express antiFL(FITC-E2) specific chimeric antigen receptor (CAR) in combination with UB-TT170, a conjugate of folate and fluorescein (FL).

Response to combination treatment with antiFL(FITC-E2) specific chimeric antigen receptor in combination with UB-TT170 is expected to be a function of individual subject's tumor burden, T cell proliferation and cytokine production, and the degree to which the individual subject's tumor expresses FRα or FRβ. Successful implementation of an antiFL(FITC-E2) CAR T cells and UB-TT170 dosing paradigm has the potential for use in the treatment of other FR-expressing cancer, including cancers of the ovary, lung as well as other malignancies.

### EC17 Human Pharmacokinetics, Dose Justification and Prior Experience

The plasma pharmacokinetics of EC17 were determined following a single, 10-minute intravenous infusion of 0.1 mg/kg in ovarian cancer subjects (n=13) prior to surgery. Blood sampling was performed pre-dose, and at 15, 30, 45 and 60 min after administration of EC17, and hourly thereafter until the end of surgery. Immediately following collection, the blood samples were put on ice and protected from light, and plasma separated and stored at −20° C. until analysis. Bioanalysis was performed using liquid chromatography with tandem mass spectrometry (LC-MS/MS) with a lower limit of quantification (LLOQ) and an upper limit of quantification (ULOQ) of 2.00 and 500 ng/ml, respectively.

The biodistribution and excretion profile of EC17 in subjects from this study was characterized by the rapid distribution and specific uptake in folate receptor positive cancerous lesions followed by a rapid clearance from circulation. The maximal plasma concentration (Cₘₐₓ) was reached at the end of the 10-minute infusion and declined thereafter, with an elimination half-life of 86.8 minutes. The EC17 fluorescence signal at the site of the tumor was observed up to 5 hours after administration of the compound, which was the latest visualization time-point evaluated surgically. The short terminal half-life in circulation, and the low background uptake, allowed for the real-time visualization of malignant lesions within 2 hours post EC17 administration. This short half-life further supports the planned treatment administration schedule that is proposed in this study.

EC17 binds tumor cell surface FR with sub-nanomolar affinity (Internal Report Endo 061401). It has good water solubility, is intrinsically small in size, and can penetrate solid tumors easily. Optical imaging studies have shown that EC17 can efficiently deliver the attached fluorescein molecule to virtually all malignant cells in both primary and metastatic tumor sites, while maintaining a sharp contrast between tumor cells and the surrounding normal tissues. With folate-imaging agents that are similar in size to EC17, a dose of ~2000 nmol/kg in mice appears to achieve FR saturation in vivo regardless of the levels of FRs on a tumor. Based on studies in mice, the therapeutically effective dose of EC17 ranges between 500 and 2000 nmol/kg to achieve opsonization of the marked tumor cells without competitive binding of circulating antibodies by excess EC17 (Internal Report 0003-PR-0010, Internal Report 0003-PR-0012)[174]. Since the affinity of FRs in mouse and human are similar, it was anticipated that saturating of the tumor-associated FRs in humans would be achieved in the clinic at a dose of 3.1×10⁻² to 1.24×10⁻¹ mg/kg of EC17, converted based on body surface area. EC17 doses of 3.1×10⁻² and 2.76×10⁻¹ mg/kg have been administered in previously-conducted human studies and the Maximum Tolerated Dose (MTD) was not reached. EC17 doses of <1.55×10⁻¹ mg/kg may be used in this study, well below previous EC17 doses found to be safe in previous human studies.

The safety of EC17 administered as a single agent in humans has been assessed in multiple exploratory pilot and Phase 2 studies. Six published studies evaluated if EC17 was effective and safe for intraoperative imaging of tumors in 84 subjects with renal, ovarian, breast or lung carcinomas. The most common adverse event reported in 10.7% of the subjects (9/84) was a mild hypersensitivity reaction, with symptoms of hives, abdominal discomfort, itching throat and sneezing. Other adverse events following intravenous EC17 administration were vomiting, abdominal discomfort, and mild injection site reaction. No serious adverse events were reported and symptoms were well controlled using supportive care measures such as administration of diphenhydramine. Overall, intravenous injection of EC17 was well tolerated.

Enrolling pediatric subjects in this study will be in accordance with 21 CFR 50, Subpart D. The initial subject enrolled onto each Dose Regimen will be ≥18 years of age and all other patients will beat least 15 years of age to allow for assent/consent from all subjects and to maximize our ability to assess for toxicity. Importantly, the Principle of Scientific Necessity per 21 CFR 50, Subpart D is met as pediatric subjects with recurrent/refractory osteosarcoma have tumor biology and anatomic locations that are distinct from other solid tumors occurring in adult patients. The peak age of osteosarcoma diagnosis is 13-16 years of age and more than half of all cased diagnosed annually occur in patients less than 20 years of age. Therefore, the age range selected for this trial is absolutely necessary to answer an important scientific question about the health and welfare of children with osteosarcoma. Furthermore, all enrolled subjects will be a target population of children with refractory or recurrent osteosarcoma for whom there are no known curative options. In regards to Equitable Selection, enrollment of children is essential to answer the scientific objectives. We will enforce justice and fair subject selection according to the Declaration of Helsinki (paragraph 24). Extrapolation from adult populations with other solid tumors is not possible due to the differences in disease biology, behavior, and anatomical tumor location as noted above. In this clinical trial, we have appropriately balanced risk and benefit and enforce additional protections for children as per 21 CFR 50, Subpart D. We have minimized risks to subjects by requiring only research procedures that directly contribute to the scientific objectives. Regarding the 21 CFR 50.52 standard of “focus”, while intravenous dosing of CAR T cells in children carries more than minimal risk, the eligibility criteria are clear in ensuring that these subjects have failed all standard of care options, providing a possible benefit. Finally, the anticipated potential benefit and possible risk will be discussed in depth with each subject and adequate provisions will be made for soliciting the assent of the subject.

### Drug Information

Investigational product for SCRI-E2CAR_EGFRtv1 is composed of autologous CD4+ and CD8+ T cells that have been genetically modified to express antiFL(FITC-E2) which is an anti-fluorescein CAR. The investigational UB-TT170 drug product (BB-IND 010704) is supplied in sterile, non-pyrogenic single-use vials for intravenous injection through a peripheral or central line over a period of 5 minutes.

Investigational Product is defined as, “A pharmaceutical form of an active ingredient or placebo being tested or used as a reference in a clinical trial, including a product with a marketing authorization when used or assembled (formulated or packaged) in a way different from the approved form, or when used for an unapproved indication, or when used to gain further information about an approved use” (from International Conference on Harmonization of Technical Requirements for Registration of Pharmaceuticals for Human Use [ICH] Harmonized Tripartite Guideline E6: Guideline for Good Clinical Practice.

The terms “Investigational Product” and “study drug” may be used interchangeably in the protocol.

### Dosing Regimen

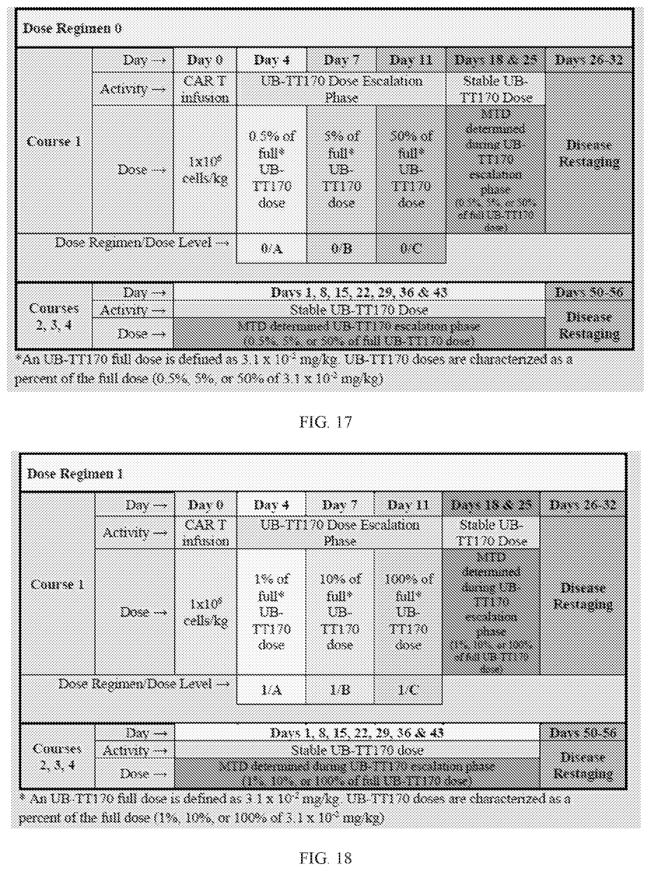

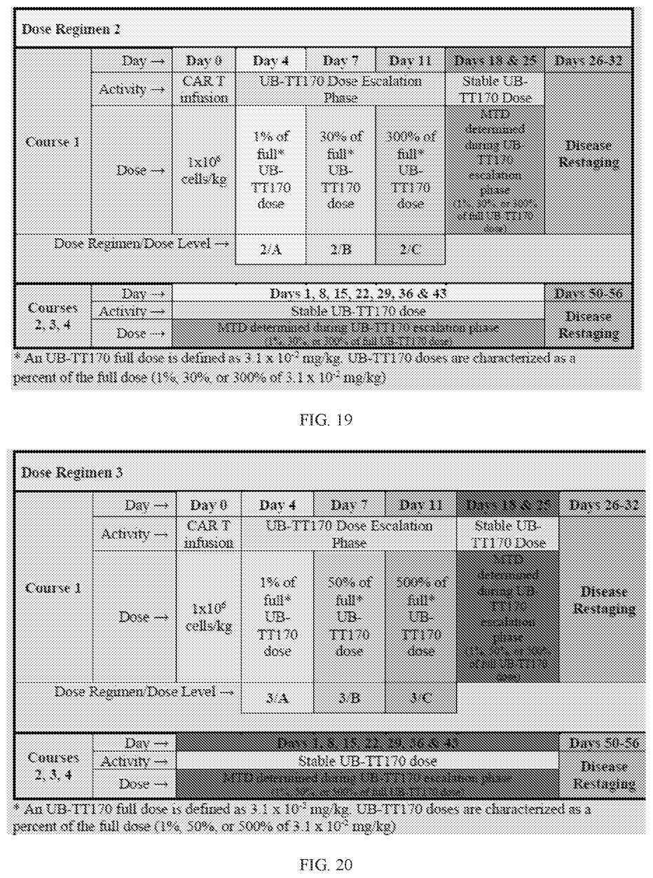

Subjects will be assigned to a Dose Regimen prior to CAR T cell product administration. Nonlimiting examples of Dose Regimens are Dose Regimen 0, 1, 2, and 3, as depicted in FIG. 17, FIG. 18, FIG. 19 and FIG. 20 and as described below. Each Dose Regimen will consist of a single, fixed, weight-based CAR T cell dose administration (Course 1, Day 0) followed by a Dose Regimen-specific UB-TT170 dose escalation sequence (Course 1, Days 4, 7 and 11), followed by a fixed, weight-based weekly dose of UB-TT170 (Course 1, Days 18 and 25).

A full (100%) dose of UB-TT170 is 3.1×10⁻² mg/kg. Each dose described in a Dose Regimen is noted as a percentage of the full (100%) dose. See TABLE 4.

TABLE 4

| UB-TT170 | | |
|---|---|---|
| percent of | Dose regimen/ | UB-TT170 dose |
| full dose | dose level | (mg/kg) |
| 0.5% | 0/A | 1.55 × 10⁻⁴ |
| 1% | 1/A or 2/A or 3/A | 3.1 × 10⁻⁴ |
| 5% | 0/B | 1.55 × 10⁻⁴ |
| 10% | 1/B | 3.1 × 10⁻⁴ |
| 30% | 2/B | 9.3 × 10⁻⁴ |
| 50% | 0/C or 3/B | 1.55 × 10⁻⁴ |
| 100% | 1/C | 3.1 × 10⁻⁴ |
| 300% | 2/C | 9.3 × 10⁻⁴ |
| 500% | 3/C | 15.5 × 10⁻⁴ |

The initial subject cohort will enroll onto Dose Regimen 1 and escalate in subsequent cohorts through Dose Regimen 3. Should Dose Regimen 1 be deemed intolerable subsequent subjects will enroll onto Dose Regimen 0. UB-TT170 dose escalation sequence will proceed in subjects who are without dose limiting toxicity (DLT). If the dose escalation phase is tolerated without DLT, subjects will receive the maximum tolerated Dose Regimen UB-TT170 dose level for subsequent UB-TT170 doses.

Dose Regimen 0 will only be utilized should Dose Regimen 1 be deemed intolerable. If subjects do accrue to Dose Regimen 1, then following CAR T cell product administration on Course 1, Day 0 subjects will enter the UB-TT170 Dose Escalation Phase. On Course 1, Day 4 subjects will receive 0.5% of the full UB-TT170 dose (Dose Level 0/A), followed by 5% of the full UB-TT170 dose on Course 1, Day 7 (Dose Level 0/B), and on Course 1, Day 11 subjects will receive 50% of the full UB-TT170 dose (Dose Level 0/C).

UB-TT170 dose escalation during the Escalation Phase can only proceed if subjects are without dose limiting toxicity (DLT) or study-significant adverse effects (ssAEs). If dose escalation is tolerated without DLT or ssAEs, subjects will receive 509% of the full UB-TT170 dose on Course 1, Days 18 and 25 and for subsequent course UB-TT170 dosing.

Dose Regimen 1 will be the initial Dose Regimen employed. Following CAR T cell product administration on Course 1, Day 0 subjects will enter the UB-TT170 Dose Escalation Phase. On Course 1, Day 4 subjects will receive 1% of the full UB-TT170 dose (Dose Level 1/A), followed by 10% of the full UB-TT170 dose on Course 1, Day 7 (Dose Level 1/B), and on Course 1, Day 11 subjects will receive 100% of the full UB-TT170 dose (Dose Level 1/C). UB-TT170 dose escalation during the Escalation Phase can only proceed if subjects are without dose limiting toxicity (DLT) or ssAEs. If dose escalation is tolerated without DLT or ssAEs, subjects will receive 100% of the full UB-TT170 dose on Course 1, Days 18 and 25 and for subsequent course UB-TT170 dosing.

Dose Regimen 2. Following CAR T cell product administration on Course 1, Day 0 subjects will enter the UB-TT170 Dose Escalation Phase. On Course 1, Day 4 subjects will receive 1% of the full UB-TT170 dose (Dose Level 2/A), followed by 30% of the full UB-TT170 dose on Course 1, Day 7 (Dose Level 2/B), and on Course 1, Day 11 subjects will receive 300% of the full UB-TT170 dose (Dose Level 2/C). UB-TT170 dose escalation during the Escalation Phase can only proceed if subjects are without dose limiting toxicity (DLT) or ssAEs. If dose escalation is tolerated without DLT or ssAEs, subjects will receive 300% of the full UB-TT170 dose on Course 1, Days 18 and 25 and for subsequent course UB-TT170 dosing.

Dose Regimen 3. Following CAR T cell product administration on Course 1, Day 0 subjects will enter the UB-TT170 Dose Escalation Phase. On Course 1, Day 4 subjects will receive 1% of the full UB-TT170 dose (Dose Level 3/A), followed by 50% of the full UB-T 170 dose on Course 1, Day 7 (Dose Level 3/B), and on Course 1, Day 11 subjects will receive 500% of the full UB-TT170 dose (Dose Level 3/C). UB-TT170 dose escalation during the Escalation Phase can only proceed if subjects are without dose limiting toxicity (DLT) or ssAEs. If dose escalation is tolerated without DLT or ssAEs, subjects will receive 300% of the full UB-TT170 dose on Course 1, Days 18 and 25 and for subsequent course UB-TT170 dosing.

For any given dose regimen, subsequent dose escalation sequences can follow a similar sequence, as depicted in FIG. 17, FIG. 18, FIG. 19, and FIG. 20. For each of additional course, the escalation dose is repeated about 7 days apart (days 1, 8, 15, 22, 29, 36, and 43). The patient can receive as many additional dose courses as are necessary; for example, the patient may receive one, two, three, four, six, eight, or ten courses of therapy.

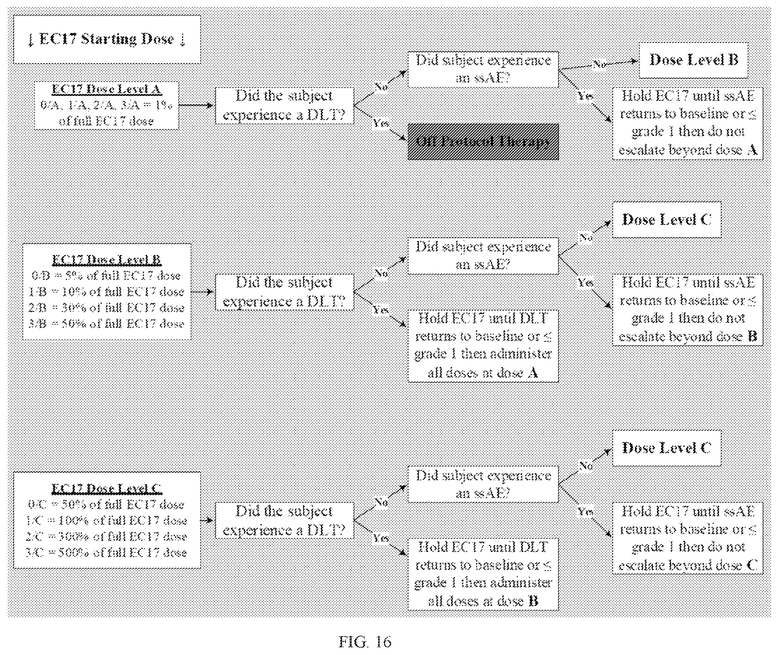

Dose modifications will be made if at any point the subject develops a toxicity that meets the definition of DLT. FIG. 16 depicts a dose modification schema.

The maximum tolerated dose regimen is defined as the highest Dose Regimen/Cohort from among those tested during the trial whose cumulative DLT rate during Course 1 is closest to 20%, provided that it is also lower than 30% and that at least 6 subjects have been evaluated at that dose cohort. A dose-limiting toxicity (DLT) is an event definitely, possibly or probably attributable to the CAR T-cell or UB-TT170 infusion and which occurs within 28 days following the CAR T cell and 7 days following the final UB-TT170 infusion. Only DLTs occurring within 28 days following the CAR T cell and 7 days following the final UB-TT170 infusion during Course 1 of each dose regimen will be used for purposes of MTDR definition and Dose Regimen escalation or de-escalation.

If no DLT and no ssAEs are observed and subject meets criteria for subsequent UB-TT170 infusion during the dose escalation phase, the subject will receive UB-TT170 Dose Level C for the remaining Course 1 UB-TT170 doses and all Course 2-4 UB-TT170 doses.

If no DLT occurs but the subject experiences an ssAE, there will be a hold on subsequent UB-TT170 infusions until the ssAE has returned to baseline or ≤Grade 1, then all subsequent UB-TT170 doses will be administered at or below the dose level that provoked the ssAE.

If a DLT occurs after the subject receives the lowest UB-TT170 dose on each Dose Regimen (Dose A), the subject should be taken off protocol therapy and no further doses of UB-TT170 should be given.

If DLT is provoked by UB-TT170 Dose B or Dose C, there will be a hold subsequent UB-TT170 infusions until the DLT has returned to baseline or ≤Grade 1. Then, all subsequent UB-TT170 doses will be administered at a dose level that is below the dose that provoked the DLT. An outline of these policies is as shown in FIG. 16.

### Disease Response

All tumor measurements will be recorded in millimeters (or decimal fractions of centimeters). Previously irradiated lesions may be considered measurable and followed for response but must demonstrate clear progression following completion most recent radiotherapy or have biopsy confirmed disease following radiotherapy. Serial measurements should be performed with the same modality (e.g., CT, MRI, PET) at each assessment. Response criteria include those listed in TABLE 5.

TABLE 5

| Response class | Criteria |
|---|---|
| Complete Response | Disappearance of all target and non-target lesions. |
| (CR) | |
| Partial Response (PR) | Disease measurement decreased by ≥30% from |
| | study enrollment. No new lesions or progression |
| | of any non-target measurable lesion. |
| Stable Disease (SD) | Criteria for CR, PR, or PD are not met. |
| Progressive Disease | Greater than 20% increase in a least one |
| (PD) | dimension of soft tissue mass documented by |
| | CT/MRI AND a minimum absolute increase of 5 |
| | mm in longest dimension in existing lesions; OR |
| | new site(s) of disease documented by CT/MRI, |
| | functional nuclear imaging or biopsy confirmed. |
| Immune Related | Radiographic evidence of progression and the |
| Progressive disease | bulleted criteria below are met: the subject is |
| (irPD) | tolerating the study drug(s) and has not |
| | experienced a DLT; and in the judgment of the |
| | treating clinician, continued treatment with |
| | UB-TT170 will not delay an imminent |
| | intervention required to prevent a serious |
| | complication (e.g., CNS metastases which require |
| | radiation therapy or surgery). |

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1-34. (canceled)

35. A method of treating, ameliorating or inhibiting a folate-receptor expressing cancer in a human subject, the method comprising administering a fluorescein isothiocyanate (FITC)-folate conjugate, or a pharmaceutically acceptable salt thereof, to a subject in an escalating dosing regimen,
wherein the escalating dosing regimen comprises a dosing escalation cycle to identify a maximum tolerated dose (MTD) of the FITC-folate conjugate, or a pharmaceutically acceptable salt thereof,
wherein the FITC-folate conjugate, or the pharmaceutically acceptable salt thereof, is administered to the subject in the escalating dosing regimen subsequent to or concurrently with administration of a chimeric antigen receptor (CAR) T cell therapy to the subject.

36. The method of claim 35, wherein the dosing escalation cycle comprises administering:
(a) a first dose of the FITC-folate conjugate, or a pharmaceutically acceptable salt thereof;
(b) a second dose of the FITC-folate conjugate, or the pharmaceutically acceptable salt thereof, wherein the second dose is higher than the first dose; and
(c) a third dose of the FITC-folate conjugate, or the pharmaceutically acceptable salt thereof, wherein the third dose is higher than the second dose, and wherein a maximum tolerated dose (MTD) of the FITC-folate conjugate, or a pharmaceutically acceptable salt thereof, is identified after the third dose; and
wherein the method further comprises:
(d) administering of the MTD of the FITC-folate conjugate, or a pharmaceutically acceptable salt thereof.

37. The method of claim 36, further comprising administering a fifth dose of the MTD of the FITC-folate conjugate, or a pharmaceutically acceptable salt thereof.

38. The method of claim 37, further comprising a restaging period after the end of the fifth dose, wherein the restaging period comprises assessing the subject to determine if one or more criteria for receiving at least one subsequent dosing cycle of the FITC-folate conjugate, or the pharmaceutically acceptable salt thereof, is met;
wherein the subject is not administered the FITC-folate conjugate during the restaging period; and
wherein if the subject meets one or more of the criteria for receiving at least one subsequent dosing cycle of the FITC-folate conjugate, or the pharmaceutically acceptable salt thereof, the subject is administered at least one subsequent dosing cycle comprising administering the MTD of the FITC-folate conjugate, or the pharmaceutically acceptable salt thereof, in a dosing interval of once every about 5-10 days for a duration of time.

39. The method of claim 35, wherein the dosing escalation cycle comprises administering:
(a) a first dose of about 0.5% to about 5% of a full dose of the FITC-folate conjugate, or a pharmaceutically acceptable salt thereof,
(b) a second dose of about 5% to about 50% of the full dose of the FITC-folate conjugate, or a pharmaceutically acceptable salt thereof, and
(c) a third dose of about 50% to about 500% of the full dose of the FITC-folate conjugate, or a pharmaceutically acceptable salt thereof, wherein the MTD is identified after the third dose, and
wherein the method further comprises:
(d) administering a fourth dose of the MTD of the FITC-folate conjugate, or a pharmaceutically acceptable salt thereof,
wherein the full dose of the FITC-folate conjugate, or a pharmaceutically acceptable salt thereof, is about 1×10⁻² mg/kg to about 5×10⁻² mg/kg.

40. The method of claim 39, further comprising administering a fifth dose of the MTD of the FITC-folate conjugate, or a pharmaceutically acceptable salt thereof.

41. The method of claim 40, further comprising a restaging period after the end of the fifth dose, wherein the restaging period comprises assessing the subject to determine if one or more criteria for receiving at least one subsequent dosing cycle of the FITC-folate conjugate, or the pharmaceutically acceptable salt thereof, is met;
wherein the subject is not administered the FITC-folate conjugate during the restaging period.

42. The method of claim 41, wherein if the subject meets one or more of the criteria for receiving at least one subsequent dosing cycle of the FITC-folate conjugate, or the pharmaceutically acceptable salt thereof, the subject is administered at least one subsequent dosing cycle comprising administering the MTD of the FITC-folate conjugate, or the pharmaceutically acceptable salt thereof, in a dosing interval of once every about 5-10 days for a duration of time.

43. The method of claim 35, wherein the dosing escalation cycle comprises administering:
(a) a first dose of about 0.5% to about 5% of a full dose of the FITC-folate conjugate, or a pharmaceutically acceptable salt thereof,
(b) a second dose of about 5% to about 50% of the full dose of the FITC-folate conjugate, or a pharmaceutically acceptable salt thereof, and
(c) a third dose of about 50% to about 500% of the full dose of the FITC-folate conjugate, or a pharmaceutically acceptable salt thereof,
wherein the full dose of the FITC-folate conjugate or a pharmaceutically acceptable salt thereof, is about 1×10⁻² mg/kg to about 5×10⁻² mg/kg; and
the method further comprises a stable dosing cycle comprising administering the MTD of the FITC-folate conjugate, or a pharmaceutically acceptable salt thereof, in a dosing interval of once every about 5-10 days for a duration of time.

44. The method of claim 35, wherein the CAR T cell therapy comprises a CAR T cell composition and the method comprises administering a dose of the CAR T cell composition that is between about 1×10⁵ cells/kg to about 1×10⁷ cells/kg.

45. The method of claim 35, wherein the cancer is an osteosarcoma.

46. The method of claim 36, wherein the period of time between administering the first dose and administering the second dose is between about 1 to about 7 days.

47. The method of claim 36, wherein the period of time between administering the second dose and administering the third dose is between about 1 to about 7 days.

48. The method of claim 36, wherein the full dose of the FITC-folate conjugate, or the pharmaceutically acceptable salt thereof, is at or about 3.1×10⁻² mg/kg.

49. The method of claim 35, wherein the CAR comprises a fully humanized anti-fluorescein or anti-FITC antibody or an antigen binding fragment thereof.

50. The method of claim 35, wherein the CAR comprises a fully humanized anti-fluorescein or anti-FITC scFv.

51. The method of claim 35, wherein the FITC-folate conjugate, or the pharmaceutically acceptable salt thereof, is administered intravenously.

52. A method of treating, ameliorating or inhibiting a folate-receptor expressing cancer in a human subject, the method comprising administering a fluorescein isothiocyanate (FITC)-folate conjugate, or a pharmaceutically acceptable salt thereof to a subject in an escalating dosing regimen,
wherein the escalating dosing regimen comprises a dosing escalation cycle to identify a maximum tolerated dose (MTD) of the FITC-folate conjugate, or a pharmaceutically acceptable salt thereof,
wherein the dosing escalation cycle comprises:
(a) a first dose comprising about 0.5% to about 5% of a full dose of the FITC-folate conjugate, or a pharmaceutically acceptable salt thereof,
(b) a second dose comprising about 5% to about 50% of the full dose of the FITC-folate conjugate, or a pharmaceutically acceptable salt thereof, and
(c) a third dose comprising about 50% to about 500% of the full dose of the FITC-folate conjugate, or a pharmaceutically acceptable salt thereof, and
wherein the method further comprises:
(d) administering a fourth dose of the MTD of the FITC-folate conjugate, or a pharmaceutically acceptable salt thereof,
wherein the period of time between administering the first dose and administering the second dose is between about 1 to about 7 days;
wherein the period of time between administering the second dose and administering the third dose is between about 1 to about 7 days;
wherein the full dose of the FITC-folate conjugate, or a pharmaceutically acceptable salt thereof, is about 1×10⁻² mg/kg to about 5×10⁻² mg/kg or 0.05 mg to about 30 mg; and
wherein the FITC-folate conjugate, or a pharmaceutically acceptable salt thereof, is administered to the subject subsequent to or concurrently with administration of the CAR T cell therapy to the subject, wherein the CAR comprises a fully humanized anti-fluorescein or anti-FITC scFv.

53. The method of claim 52, wherein:
in (a) the first dose is about 0.5% or about 1% of a full dose of the FITC-folate conjugate, or a pharmaceutically acceptable salt thereof,
in (b) the second dose is about 5%, about 10%, about 30%, or about 50% of the full dose of the FITC-folate conjugate, or a pharmaceutically acceptable salt thereof, and
in (c) the third dose is about 50%, about 100%, about 300%, or about 500% of the full dose of the FITC-folate conjugate, or a pharmaceutically acceptable salt thereof.

54. The method of claim 35, wherein:
in (a) the first dose is about 0.5% or about 1% of a full dose of the FITC-folate conjugate, or a pharmaceutically acceptable salt thereof,
in (b) the second dose is about 5%, about 10%, about 30%, or about 50% of the full dose of the FITC-folate conjugate, or a pharmaceutically acceptable salt thereof, and
in (c) the third dose is about 50%, about 100%, about 300%, or about 500% of the full dose of the FITC-folate conjugate, or a pharmaceutically acceptable salt thereof.
